(19) United States
(12) Patent Application Publication
(10) Pub. No.: US 2023/0340071 A1
(43) Pub. Date: Oct. 26, 2023

(54) Interleukin-2 Receptor Beta (IL-2RB) Binding Polypeptides

(71) Applicants: Neoleukin Therapeutics, Inc., Seattle, WA (US); University of Washington, Seattle, WA (US)

(72) Inventors: Daniel Adriano Silva Manzano, Seattle, WA (US); Alfredo Quijano Rubio, Seattle, WA (US); Jesus Renan Vergara Gutierrez, Seattle, WA (US); Thomas Linsky, Seattle, WA (US); Jorgen Nelson, Seattle, WA (US)

(73) Assignees: Neoleukin Therapeutics, Inc., Seattle, WA (US); University of Washington, Seattle, WA (US)

(21) Appl. No.: 17/908,844

(22) PCT Filed: Mar. 12, 2021
(86) PCT No.: PCT/US2021/022075

§ 371 Date: Sep. 1, 2022

Related U.S. Application Data
(60) Provisional application No. 62/990,177, filed on Mar. 16, 2020.

(51) Int. Cl. C07K 14/715

(52) U.S. Cl. CPC C07K 14/7155; A61K 38/00

(57) ABSTRACT

The present invention is related to, inter alia, de novo IL-2R13 binding polypeptides.

### CROSS-REFERENCE TO RELATED APPLICATIONS

This application claims the benefit of priority of U.S. Provisional Application No. 62/990,177, filed Mar. 16, 2020, which is incorporated by reference herein in its entirety for any purpose.

### FIELD

The present invention is related to, inter alia, de novo IL-2Rβ binding polypeptides.

### BACKGROUND

A de novo protein immunotherapeutic, Neoleukin-2/15 (also known as Neo-2/15), has recently been described. Neo-2/15 is a de novo protein mimic of the function of both human interleukin-2 (hIL-2) and human interleukin-15 (hIL-15). To accomplish its biological function, Neo-2/15 induces the hetero-dimerization of two IL-2 cell membrane receptors, the IL-2 receptor beta (IL-2R13) and the IL-2 receptor common gamma IL-2Rγ_{c}. The IL-2 heterodimeric receptor is also known as IL-2Rβγ_{c}. The hetero-dimerization of IL-2Rβγ_{c} caused by hIL-2 (and also Neo-2/15), initiates a signaling cascade that is responsible for stimulating the activation and proliferation of several types of immune cells (such as T-cells, among others). Unlike IL-2, Neo-2/15 signals independently of CD25 and unlike IL-15, it also signals independently of CD-215. Neo-2/15 is highly thermostable protein that demonstrates potent IL-2 like signaling on both human and mouse cells. Neo-2/15 has been shown to have anti-cancer therapeutic activity in several murine models. Conversely, for other diseases, inhibiting the signaling cascade induced by dimerization of IL-2Rβγ_{c} is desirable, and several studies have sought to exploit such a paradigm to combat inflammation-related disease using IL-2R antibodies. These therapeutic regimens have met limited success. The identification of de novo proteins that can specifically and potently block IL-2 and/or IL-15 signaling involved in inflammation and autoimmune disease has the potential to translate into successful clinical candidates that can be used for a multitude of diseases, including those of autoimmune nature. The present disclosure addresses this and other needs.

### DRAWINGS

FIG. 1A-1B: FIG. 1A demonstrates the binding of Neo-2/15, P3 and P4 to IL-2Rγ_{c} as measured by biolayer interferometry. FIG. 1B demonstrates the pSTAT5 signaling elicited by increasing concentrations of PEGylated Neo-2/15 (black triangles), P3 (filled in circles), P4 (open circles) in human Pan T cells.

FIGS. 2A-2C demonstrates human IL-2 pSTAT5 signaling inhibition by P3 (filled-in circles) and P4 (open circles) at different concentrations of human IL-2.

FIGS. 3A-3C show biolayer interferometry (OCTET) binding assays of S4 (3A), P5 (3B), and P6 (3C) against immobilized hIL-2Rβ.

FIGS. 4A-4C show biolayer interferometry (OCTET) binding assays of S4 (4A), P5 (4B), and P6 (4C), in complex with hIL-2Rβ, against immobilized hIL-2Rγ_{c}.

FIGS. 5A-5C show the binding inhibition of hIL-2 to hIL-2Rβγ_{c} by S4 (5A), P5 (5B), and P6 (5 C).

FIGS. 6A-6D show percentage IL-2R pSTAT5 signaling inhibition on all T cells (6 A), CD4+ T cells (6B), CD8+ T cells (6C) and Regulatory T cells (6D) by P5, P6, S4, and an anti-IL-2 IgG.

FIGS. 7A-7B show the pSTAT5 signaling ability of P6 (7A) and P5 (7B) on all T cells, CD4+ T Cells, CD8+ T Cells and Regulatory T cells.

FIGS. 8A-8 D show the pSTAT5 signaling ability of S4 on Regulatory T cells (8A), CD4+CD25− T cells (8B), CD4+CD25+ T cells (8C) and CD8+ T cells (8D).

FIGS. 9A-9B show the pSTAT5 signaling ability of S4 on PBMC cells (9A) and NK cells (9B).

FIGS. 10A-10D show percentage IL-15R pSTAT5 signaling inhibition on all T cells (10A), CD4+ T cells (10B), CD8+ T cells (10C) and Regulatory T cells (10D) by P5, P6, S4, and an anti-IL-2 IgG.

FIGS. 11A-11C show circular dichroism (CD) of S4 (A), P5 (B), and P6 (C). Far UV wavelength spectra is shown at 20° C., after heating to about 98° C. and after cooling the heated sample to 20° C.

FIGS. 12A-12C show temperature unfolding curves for S4 (12A), P5 (12B), and P6 (12C) obtained from 20 to 98° C. by monitoring the CD signal at 222 nm.

### SUMMARY

The present inventors have identified novel methods to modulate the activity of IL-2. In some aspects, the methods are effective at reducing (i.e. inhibiting) one or more activities of IL-2 or IL-15. In some aspects, the methods are effective at blocking one or more activities of IL-2 or IL-15. In particular, the present inventors have created polypeptides that bind to IL-2Rβ but have no binding site for IL-2Rα and have reduced binding affinity (including fully ablated binding) to IL-2Rγ_{c} (as compared to IL-2). In some aspects, the polypeptides of the present invention have substantially the same or increased binding affinity to IL-2Rβ as compared to Neo-2/15. In some aspects, the polypeptides of the present invention have substantially the same binding affinity to IL-2Rβ as compared to IL-2. In some particularly preferred aspects, the polypeptides of the present invention have increased binding affinity to IL-2Rβ as compared to IL-2.

In some aspects, polypeptides of the present invention act to limit (i.e., inhibit) or prevent IL-2 from binding to and co-localizing IL-2Rβ with IL-2Ryc thereby limiting (i.e., inhibiting) or blocking the ability of IL-2 to signal through IL-2R. In such a manner, exemplary polypeptides of the present invention antagonize the activity of IL-2. Accordingly, in some aspects, the polypeptides of the present invention act as antagonists of the biological function of IL-2. Polypeptides that act as antagonists of the biological function of IL-2 by competing for the IL-2 receptor can also be referred to as IL-2R antagonists.

The IL-15 receptor shares two signaling subunits with the IL-2 receptor, namely IL-2Rβ and IL-2Rγ_{c} . In some aspects, exemplary polypeptides of the present invention act to limit (i.e., inhibit) or prevent IL-15 from binding to the shared IL-2Rβγ_{c} . Accordingly, in some aspects, the polypeptides of the present invention act as antagonists of the biological function of IL-15. Polypeptides that act as antagonists of the biological function of IL-15 by competing for the IL-15 receptor can also be referred to as IL-15R antagonists. Exemplary polypeptides of the present invention inhibit the binding of IL-2 to the IL-2 receptor and/or signaling via the IL-2 receptor in select IL-2Rβ positive cell types. In some embodiments, IL-2Rβ positive cell types are IL-2Rγ_{c} positive but are either IL-2Ra positive or IL-2Rα negative. In some embodiments, polypeptides of the present invention inhibit the binding of IL-2 to the IL-2 receptor and/or signaling via the IL-2 receptor to a greater extent in cells that are IL-2Rα negative as compared to cells that are IL-2Rα positive. In some embodiments, polypeptides of the present invention inhibit the binding of IL-2 to the IL-2 receptor and/or signaling via the IL-2 receptor by at least about 30%, at least about 40%, at least about 50%, at least about 60%, at least about 70%, at least about 80%, at least about 90%, or at least about 95% in IL-2Rβ positive cells that are IL-2Rα negative. In some embodiments, polypeptides of the present invention inhibit the binding of IL-2 to the IL-2 receptor and/or signaling via the IL-2 receptor by at least about 30%, at least about 40%, at least about 50%, at least about 60%, at least about 70%, at least about 80%, at least about 90%, or at least about 95% in IL-2Rβ positive cells that are IL-2Rα negative and by not more than 50%, not more than 30% or by not more than 20% in IL-2Rβ positive cells that are IL-2Rα positive.

In some embodiments, polypeptides of the present invention have limited ability or no ability themselves to induce the heterodimerization or dimerization of IL-2βRγ_{c} and, as such, have a reduced ability (including to negligible and/or undetectable levels) to simulate STATS phosphorylation as compared to IL-2. In some embodiments, polypeptides of the present invention stimulate STATS phosphorylation at a level that is at least 50% less than the level that IL-2 stimulates STATS phosphorylation in the same type of cell. In some embodiments, polypeptides of the present invention stimulate STATS phosphorylation at a level that is at least 60% less, at least 70% less, at least 80% less, at least 85% less, at least 90% less, or at least 95% less than the level that IL-2 stimulates STATS phosphorylation in the same type of cell.

In some embodiments, polypeptides of the present invention stimulate STATS phosphorylation at negligible levels, including undetectable levels. In some embodiments, polypeptides of the present invention have a reduced ability (including to negligible and/or undetectable levels) to simulate STATS phosphorylation as compared to Neo-2/15.

Exemplary polypeptides of the present invention inhibit the ability of IL-2 and/or IL-15 to stimulate STATS phosphorylation in IL-2Rβ positive cell types. In some embodiments, polypeptides of the present invention inhibit the ability of IL-2 to stimulate STATS phosphorylation in IL-2Rβ positive cell types by at least about 30%, at least about 40%, at least about 50%, at least about 60%, at least about 70%, at least about 80%, at least about 85%, at least about 90%, or at least about 95%. In some embodiments, polypeptides of the present invention inhibit the ability of IL-2 to stimulate STATS phosphorylation by at least about 30%, at least about 40%, at least about 50%, at least about 60%, at least about 70%, at least about 80%, at least about 90%, or at least about 95% in IL-2Rβ positive cells that are IL-2Rα negative. In some embodiments, polypeptides of the present invention inhibit the ability of IL-2 to stimulate STATS phosphorylation by at least about 30%, at least about 40%, at least about 50%, at least about 60%, at least about 70%, at least about 80%, at least about 90%, or at least about 95% in IL-2Rβ positive cells that are IL-2Rα negative and by not more than 50%, not more than 40%, not more than 30% or by not more than 20% in IL-2Rβ positive cells that are IL-2Rα positive.

Polypeptides of the present invention were created using the backbone of the de novo protein Neo-2/15 and, as such, possess the advantages of de novo proteins. One such advantage is increased stability as compared to native proteins and derivatives thereof.

Also provided herein are pharmaceutical compositions comprising polypeptides of the present invention and pharmaceutically acceptable carriers; as well as methods of using such polypeptides and pharmaceutical compositions.

Methods of antagonizing IL-2R and/or IL-15R are provided herein. Such methods comprise administering to a subject at least one polypeptide of the present invention. Also provided herein are methods of modulating IL-2 and/or IL-15 activity in a subject comprising administering to a subject at least one polypeptide of the present invention. Methods of treating diseases associated with IL-2 and/or IL-15 activity in a subject are also provided. The subject can be a mammalian subject. In some embodiments, the subject is a non-human primate or a human.

Methods of making the polypeptides of the present invention are further described by using nucleic acids encoding the polypeptides, expression vectors comprising the nucleic acids, and recombinant host cells.

IL-2, IL-15 and the IL-2 receptor can be from a mammalian source. In any of the embodiments decribed herein, IL-2, IL-15 and the IL-2 receptor can be human IL-2, human IL-15 and/or the human IL-2 receptor. In some embodiments, the IL-2 receptor is the mouse IL-2 receptor. In some aspects, the IL-2 receptor is the human IL-2 receptor.

### DESCRIPTION

As used herein, “IL-2” refers to native wild-type IL-2 or recombinant IL-2. “Human IL-2” or “hIL-2” refers to native wild-type human IL-2 or recombinant IL-2 (rhIL2, or simply rIL-2, or hIL-2). The amino acid sequence of native human wild-type IL-2 is found in the Genbank under accession locator NP 000577.2 and is as set forth in SEQ ID NO:26. (SEQ ID NO:26—MYRMQLLSCIALSLALVTNS APTSSSTKKTQLQLEHLLLDLQMILNGINN YKNPKLTRMLTFKFYMPKKATELKHLQCLEEELKPLEEVLNLAQSKNFHL RPRDLISNINVIVLELKGSETTFMCEYADETATIVEFLNRWITFCQSIISTLT).

The amino acid sequence of mature native human wild-type IL-2 lacks the N-terminal 20 amino acid signal peptide. An exemplary recombinant form of IL-2 does not have the N terminal alanine of wild-type IL-2 and serine is substituted for cysteine at amino acid position 125.

“De novo IL-2 protein mimics” refer to the de novo IL-2 protein mimics described in Silva et al., De novo design of potent and selective mimics of IL-2 and IL-15, Nature 2019, 565:186. As used herein, “Neo-2/15” refers to the de novo protein mimic Neo-2/15 described in Silva et al. The amino acid sequence of Neo-2/15 is as set forth in SEQ ID NO:25. (SEQ ID NO:25—PKKKIQLHAEHALYDALMILNIVKTNSPPAEEKLEDYAFNFELILEEIARLFESGDQKD EAEKAKRMKEWMKRIKTTASEDEQEEMANAIITILQSWIFS).

“Numbered in accordance with Neo-2/15” or “according to the numbering of Neo-2/15” means identifying an amino acid with reference to the position at which that amino acid occurs in the sequence of Neo-2/15, for example L17 refers to the seventeenth amino acid, leucine, that occurs in SEQ ID NO: 25.

It will be noted throughout the application, that in certain instances (e.g., For SEQ ID NO:7), solely for the purpose of maintaining consistency throughout the application with respect to position numbering, an amino acid at the N terminus of a sequence is designated as position 4 (e.g., the lysine at the N terminus of SEQ ID NO:7). Despite the designation as position 4, the polypeptide need not, but optionally may, comprise amino acids N-terminal to the amino acid designated as position 4. In other aspects, an amino acid at the N terminus of a sequence is designated as position 1. Similarly, despite the designation as position 1, the polypeptide need not, but optionally may, comprise amino acids N-terminal to the amino acid designated as position 1. If position numbering is not noted, the amino acid at the N terminus of the sequence is designated as position 1, unless context indicates otherwise.

“Affinity” or “binding affinity” refers to the strength of the sum total of non-covalent interactions between a binding site of a molecule and its binding partner. The affinity of a molecule for its partner can generally be represented by the dissociation constant (KD).

“Reduced binding” refers to a decrease in affinity for the respective interaction. The term also includes reduction of the affinity to zero (or below the detection limit of the analytic method), i.e., complete abolishment of the interaction. Conversely, “increased binding” refers to an increase in binding affinity for the respective interaction.

As used herein, the natural amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). As used herein “any amino acid” typically refers to the 20 natural amino acids. The skilled practitioner will appreciate, however, that one or more, (e.g., from 1 to 10, 1 to 5, 1 to 3, or 1 or 2) unnatural amino acids can be used in place of a natural amino acid. As used herein, the term “unnatural amino acid” refers to an amino acid other than the 20 amino acids that occur naturally in protein. Unnatural amino acids are known in the art.

The “IL-2 receptor common gamma” or “IL-2Ryc” or “ IL-2RG” refers to the IL-2 gamma receptor and is a member of the type I cytokine receptor family that is a cytokine receptor subunit to the receptor complexes for at least six different interleukin receptors including, but not limited to, IL-2, IL-4, IL-7, IL-9, IL-15, and IL-21 receptors. The “hIL-2 receptor common gamma” or “hIL-2Rγ_{c}” or “hIL-2RG” refers to the human IL-2 gamma receptor. A nucleic acid sequence of the human IL-2 gamma receptor is found in Genbank under accession locator NM_000206. An amino acid sequence of the human IL-2 gamma receptor is found in Genbank under accession locator NP_000197 and is set forth in SEQ ID NO:48:

MLKPSLPFTSLLFLQLPLLGVGLNTTILTPNGNEDTTADFFLTTMPTDS
LSVSTLPLPEVQCFVFNVEYMNCTWNSSSEPQPTNLTLHYWYKNSDNDK
VQKCSHYLFSEEITSGCQLQKKEIHLYQTFVVQLQDPREPRRQATQMLK
LQNLVIPWAPENLTLHKLSESQLELNWNNRFLNHCLEHLVQYRTDWDHS
WTEQSVDYRHKFSLPSVDGQKRYTFRVRSRFNPLCGSAQHWSEWSHPIH
WGSNTSKENPFLFALEAVVISVGSMGLIISLLCVYFWLERTMPRIPTLK
NLEDLVTEYHGNFSAWSGVSKGLAESLQPDYSERLCLVSEIPPKGGALG
EGPGASPCNQHSPYWAPPCYTLKPET.

“IL-2Rβ receptor” or “IL-2R receptor beta” refers to the IL-2 beta receptor. “hIL-2Rβ receptor” or “hIL-2R receptor beta” refers to the human IL-2 beta receptor. An amino acid sequence of the human IL-2 beta receptor is found in Genbank under accession locator NP 001333152.1 and is as set forth in SEQ ID NO:49

MAAPALSWRLPLLILLLPLATSWASAAVNGTSQFTCFYNSRANISCVWS
QDGALQDTSCQVHAWPDRRRWNQTCELLPVSQASWACNLILGAPDSQKL
TTVDIVTLRVLCREGVRWRVMAIQDFKPFENLRLMAPISLQVVHVETHR
CNISWEISQASHYFERHLEFEARTLSPGHTWEEAPLLTLKQKQEWICLE
TLTPDTQYEFQVRVKPLQGEFTTWSPWSQPLAFRTKPAALGKDTIPWLG
HLLVGLSGAFGFIILVYLLINCRNTGPWLKKVLKCNTPDPSKFFSQLSS
EHGGDVQKWLSSPFPSSSFSPGGLAPEISPLEVLERDKVTQLLLQQDKV
PEPASLSSNHSLTSCFTNQGYFFFHLPDALEIEACQVYFTYDPYSEEDP
DEGVAGAPTGSSPQPLQPLSGEDDAYCTFPSRDDLLLFSPSLLGGPSPP
STAPGGSGAGEERMPPSLQERVPRDWDPQPLGPPTPGVPDLVDFQPPPE
LVLREAGEEVPDAGPREGVSFPWSRPPGQGEFRALNARLPLNTDAYLSL
QELQGQDPTHLV.

“IL2RBG” or “IL-2Rβγ_{c}” refers to the IL-2Rβ and IL-2Rγ_{c} heterodimer. “hIL2RBG” or “hIL-2Rβγ_{c}” refers to the hIL-2Rβ and hIL-2Rγ_{c} heterodimer. IL-2Rβγ_{c} is also known as the intermediate affinity IL-2 Receptor.

The terms “polypeptide”, “protein” and “peptide” are used interchangeably to refer to any chain of amino acid residues, regardless of its length or post-translational modification (e.g., glycosylation or phosphorylation). Polypeptides of the present invention include IL-2Rβ binding proteins and Neo-2/15 variant proteins.

An “agonist” is a compound that interacts with a target to cause or promote an increase in the activation of the target.

A “partial agonist” is a compound that interacts with the same target as an agonist (and in a similar fashion/structural-mechanism) but does not produce as great a magnitude of a biochemical and/or physiological effect as the agonist at a given concentration, even by increasing the dosage of the partial agonist.

An “IL-2 antagonist” or “IL-2R antagonist” as used herein is a compound that opposes one or more actions of IL-2 or one or more activities of IL-2. The term antagonist refers to both full antagonists and partial antagonists. For example, a “partial antagonist” is an antagonist that does not-fully interrupt the biochemical effect of IL-2, but that is sufficient to interrupt selected targeted cellular and/or physiological activities promoted by IL-2. An antagonist of IL-2 might, under certain biological scenarios, have ability to induce IL-2-like signaling on its own (i.e., pSTAT5 signaling). In some embodiments, the ability to induce IL-2-like signaling will be at a lower level than the signaling induced by IL-2.

“Operably linked” is intended to mean that the nucleotide sequence of is linked to the regulatory sequence(s) in a manner that allows for expression of the nucleotide sequence (e.g., in an in vitro transcription/translation system or in a host cell when the vector is introduced into the host cell). “Regulatory sequences” include promoters, enhancers, and other expression control elements (e.g., polyadenylation signals). The expression constructs of the invention can be introduced into host cells to thereby produce the polypeptides of the present invention.

The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell but are still included within the scope of the term as used herein.

Polypeptides and polynucleotides can be provided in “isolated” form. This means that they are separated from one or more components with which they occur in nature or during production. In some aspects, an isolated polypeptide is at least 50% w/w pure of other components present during its production and/or purification but does not exclude the possibility that it is combined with an excess of pharmaceutically acceptable carrier or other vehicle intended to facilitate its use.

As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, particle gun, or electroporation.

As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics) can also be incorporated into the compositions.

### Polypeptides

The polypeptides of the present invention are modeled on Neo-2/15 but contain amino acid substitutions that interfere with binding to the IL-2 common gamma receptor. At the same time, the polypeptides retain binding to IL-2Rβ either by retaining the amino acids of Neo-2/15 at the positions responsible for binding to IL-2Rβ or by substituting select amino acids with ones that retain the polypeptides' ability to IL-2Rβ or even increase affinity for IL-2Rβ. In some embodiments, when bound to IL-2Rβ in the same site where IL2 does, the exemplary polypeptides of the present invention inhibit the binding of IL-2 to the IL-2R, thereby inhibiting IL-2's ability to induce IL-2R beta and common gamma heterodimerization and signaling via IL-2R. In some embodiments, polypeptides of the present invention have reduced ability, including no detectable ability, to induce signaling via IL-2R as compared to IL-2 in cells that express IL-2R13. In some embodiments, polypeptides of the present invention inhibit IL-2's binding to and/or signaling via the intermediate affinity IL-2 receptor complex (i.e., the heterodimer of the IL-2Rβ and the common gamma chain) to a greater degree than they inhibit IL-2's binding to and/or signaling via the high affinity IL-2 receptor complex (heterotrimer of the IL-2R beta, gamma, and alpha chains). In some embodiments, polypeptides of the present invention inhibit IL-15's binding to and/or signaling via the intermediate affinity IL-2 receptor complex (i.e., the heterodimer of the IL-2Rβ and the common gamma chain) to a greater degree than they inhibit IL-15's binding to and/or signaling via the high affinity IL-15 receptor complex (heterotrimer of the IL-2R beta, IL-2R gamma and IL-15R alpha chains). In some embodiments, exemplary polypeptides of the present invention selectively inhibit binding of IL-2 to IL-2R, i.e., they are able to inhibit IL-2 binding to a greater degree in certain cell types than in others. Accordingly, in some particularly preferred embodiments, the polypeptides of the present invention are able to selectively modulate the activity of IL-2 and its ability to bind to and signal via IL-2R. In some aspects, polypeptides of the present invention are able to inhibit IL-2's binding to and/or signaling via IL-2R to a greater degree in cell types that don't express or transiently express CD25 as compared to cells that constitutively express CD25. In some aspects, polypeptides of the present invention are able to inhibit IL-2's binding to and/or signaling via IL-2R to a greater degree in cells that don't express CD25 or express only low to medium levels of CD25 than in those cells that express high levels of CD25. In some aspects, polypeptides of the present invention are able to inhibit IL-2's binding to and/or signaling via IL-2R to a greater degree in CD4+CD25− cells and CD8+CD25- cells as compared to T regulatory cells. In some aspects, polypeptides of the present invention only minimally inhibit or don't inhibit IL-2's binding to and/or signaling via IL-2R in T regulatory cells. Because of the ability of polypeptides of the present invention to inhibit IL-2's ability to activate and induce proliferation of CD8+ and CD4+ T cells that are involved in inflammation, autoimmunity, organ graft rejection, GVHD and other disease, they are well suited to treat diseases associated with dysfunction of CD4+CD25− T cells, and CD8+CD25− T cells. In some embodiments, they can do so while having little or no inhibitory effect on IL-2's binding to and/or signaling via IL-2R in regulatory T cells. Accordingly, in some aspects, polypeptides of the present invention can be used to attenuate (e.g., inhibit or ablate) IL-2R signaling in certain cell types and not others. In some embodiments, IL-2 signaling will be attenuated (e.g., inhibited or ablated) in NK cells. In some embodiments, IL-2 signaling may be attenuated (e.g., inhibited or ablated) in CD8+CD25− T cells. In some embodiments, IL-2 signaling may be attenuated (e.g., inhibited or ablated) in CD4+CD25− T cells.

In some embodiments, polypeptides of the present invention bind IL-2Rγ_{c} with an affinity that is at least 5 fold, 10 fold, at least 20 fold, at least 50 fold, at least 100 fold, at least 1000 fold, or at least 10,000 fold or more lower than IL-2 when tested in the same assay under the same conditions. In some embodiments, exemplary polypeptides do not detectably bind to IL-2Rγ_{c}. The binding affinity of subject polypeptides for IL-2Rγ_{c} can be measured using any suitable method known in the art. Suitable methods for measuring IL-2Rγ_{c} binding, include, but are not limited to, isothermal titration calorimetry binding assays, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET) and surface plasmon resonance assays (see, e.g., Drescher et al., Methods Mol Biol 493:323-343 (2009)); liquid phase ligand binding assays (e.g., real-time polymerase chain reaction (RT-qPCR), and immunoprecipitation); and solid phase ligand binding assays (e.g., multi-well plate assays, on-bead ligand binding assays, on-column ligand binding assays, and filter assays). A preferred method for determining affinity is biolayer interferometry, for example, as described in the examples. In a particularly preferred method, binding to IL-2Rγ_{c} is measured by determining binding to IL-2Rγ_{c} in the presence of IL-2RB as shown in Example 6. In some particularly preferred embodiments, IL-2Rγ_{c} is hIL-2Rγ_{c}.

In some aspects, in order to create a strong antagonist to IL-2R, it is desirable to have increased binding affinity to IL-2Rβ as compared to IL-2. In some aspects, polypeptides of the present invention bind IL-2Rβ with substantially the same affinity as wild-type IL-2. In some aspects, polypeptides of the present invention bind IL-2Rβ with an affinity that is at least 5 fold, at least 10 fold, at least 20 fold, at least 50 fold, at least 100 fold, at least 500 fold, or at least 1000 fold or more fold stronger that that of IL-2 in the same assay under the same conditions. In some embodiments, a polypeptide of the present invention binds IL-2Rβ with a KD of 20 nM or lower, a KD of 10 nM or lower, a a KD of 5 nM or lower, or a KD of 1 nM or lower. Binding can be assessed by any suitable method known to those in the art. A preferred method for determining affinity is biolayer interferometry, for example, as described in the examples. In a particularly preferred method, binding is measured as shown in Example 6. In some particularly preferred embodiments, IL-2Rβ is hIL-2Rβ.

### Structural and Sequence Characteristics of Exemplary Polypeptides of the Present Invention

The present inventors have identified a combination of mutations that can be made to the Neo-2/15 polypeptide to result in a polypeptide that retains binding to hIL-2Rβ but has significantly reduced binding to, or no detectable binding to, hIL-2Rγ_{c}

Not only have the present inventions discovered combinations of mutations that are effective at significantly reducing binding to IL-2Rγ_{c}, resulting in polypeptides with reduced ability to induce IL-2R signaling, they have shown that by increasing the binding to IL-2Rβ while at the same time decreasing and/or abolishing binding to IL-2Rγ_{c}, they can create polypeptides with improved ability to competitively inhibit the binding of IL2 to its receptor thereby creating IL-2R antagonists. Methods of testing polypeptides of the present invention for signaling activity and/or ability to competitively inhibit IL-2 binding to IL-2R are known in the art and are described herein. One particular method of determining the ability of a polypeptide to competitively inhibit IL-2 binding to IL-2R is shown in Example 6.

Accordingly, provided herein are, inter alia, polypeptides that (i) bind to IL-2Rβ, (ii) have no binding site for IL-2Rα and (iii) have reduced binding to, or no detectable binding to, IL-2Rγ_{c}. In some embodiments, IL-2Rβ is hIL-2Rβ.

In some aspects, reduction of binding is as compared to IL-2. In some aspects, reduction of binding is as compared to Neo-2/15. In some aspects, the polypeptides have increased binding affinity to IL-2R13. In some aspects, increased binding affinity to IL-2Rβ is as compared to Neo-2/15. In some aspects, increased binding affinity to IL-2Rβ is as compared to IL-2. In some embodiments, IL-2Rβ is hIL-2Rβ.

The polypeptides of the present invention were created using the backbone of the de novo protein Neo-2/15 and, as such, are also de novo proteins, and are therefore non-naturally occurring proteins. In some embodiments, exemplary polypeptides of the present invention comprise at least 4 domains. In some embodiments, the domains are helical domains. IL-2 comprises 4 domains, and binding sites for IL-2Rα , IL-2R13, and IL-2Rγ_{c}. Neo-2/15 comprises 4 domains, including binding sites to IL-2Rβ and IL-2Rγ_{c}, but has no binding site for IL-2Rα.

In exemplary embodiments of the present invention, polypeptides of the present invention comprise 4 domains. The four domains are referred to herein as D1, D2, D3, and D4. Domains D1 and D3, as in Neo-2/15, interact with IL-2R via binding to IL-2R13. Domain D1 also interacts with IL-2R via binding to IL-2Rγ_{c}. Whereas in Neo-2/15, D4 is primarily responsible for interacting with IL-2R via binding to IL-2Rγ_{c}, in the exemplary polypeptides of the present invention, a selection of amino acid residues in D4 involved in binding to IL-2Rγ_{c} have been mutated in order to reduce binding affinity to IL-2Rγ_{c}. The D2 domain has little interaction with the IL-2R and for this reason can have a great deal of variability in its amino acid composition on the surface.

As shown in the examples herein, mutations at positions 17, 21, 89, 91, 92, 95, 96, and 99 (i.e., P2, P4/S4, P5 and P6) are effective at significantly reducing binding of the polypeptides to hIL-2Rγ_{c}. Similarly, mutations at positions 13, 17, 95, and 99 (i.e., P1, P3) can also significantly reduce binding of the polypeptides to hIL-2Rγ_{c}. In some aspects, polypeptides with a subset of the noted mutations can be made. With respect to position 21, for example, due to the distance from the gamma chain, position 21 is not believed to be involved in the reduction of binding to the gamma chain.

The polypeptides optionally comprise linkers between the domains. In some embodiments, a linker is composed of amino acids. Such linkers, including amino acid linkers, function to connect the four domains. They are typically not directly involved in binding and, for those reasons, there is great variability permitted in the length of the linker and the identity of the amino acids. In various embodiments, the linkers can be of any length. In some aspects, the linkers are from 1 to 100 amino acids in length, such as 1-100, 1-90, 1-80, 1-70, 1-60, 1-50, 1-40, 1-30, 1-20, 1-10, 2-10 or 1-5 amino acids in length. The skilled practitioner can use the teachings in the art (See, for example, Silva et al., Nature, 2019 January; 565(7738):186-191; T. W. Linsky et al., Science 10.1126/science.abe0075 (2020)) in combination with the teachings of the present specification to construct linkers for connecting the domains while maintaining the desirable properties of the polypeptides. In addition to variability in the length and identity of the amino acids, there is also a great deal of variability permitted in the ordering of the domains, D1, D2, D3, and D4. In various embodiments, the domains can be linked via amino acid linkers in varying order and still be properly folded and presented for binding to IL-2R13. As noted, the order of domains in Neo-2/15 and the exemplary polypeptides of the present invention is D1-D3-D2-D4. The skilled artisan will understand, however, that the domains can be re-ordered and still result in polypeptides having the desired activities.

Included are embodiments wherein the order of the domains is D1, D3, D2 and D4, wherein there is a first linker between domains D1 and D3, a second linker between domains D3 and D2, and a third linker between D2 and D4. In some aspects, the first linker is 10 amino acids in length, the second linker is 2 amino acids in length, and the third linker is 3 amino acids in length. An exemplary sequence for the first linker is VKTNSPPAEE (SEQ ID NO:23). An exemplary sequence for the second linker is DQ and an exemplary sequence for the third linker is TAS (SEQ ID NO:24).

### Section A

Exemplary polypeptides of the present invention bind IL-2Rβ and comprise the domains D1, D2, D3, and D4 wherein:
(a) D1 comprises the amino acid sequence: KIQLYAEHAL YDAX₁₇MILX₂₁I (SEQ ID NO:1);
(b) D2 comprises an amino acid sequence at least 8 amino acids in length;
(c) D3 comprises the amino acid sequence ELEDYAFN FELILEEIAR LFESG (SEQ ID NO:2); and
(d) D4 comprises the amino acid sequence EDEQEEMANX₈₉I X₉₁X₉₂ILX₉₅X₉₆WIX₉₉S (SEQ ID NO:3)

Exemplary polypeptides of the present invention bind IL-2Rβ and comprise the domains D1, D2, D3, and D4 wherein:
(a) D1 comprises the amino acid sequence: KIQLX₈AEHAL YDAX₁₇MILX₂₁I (SEQ ID NO:4;
(b) D2 comprises an amino acid sequence at least 8 amino acids in length;
(c) D3 comprises the amino acid sequence X₃₃LEDYAFN FELILEEIAR LFESG (SEQ ID NO:5)
(d) D4 comprises the amino acid sequence. EDEQEEMANX₈₉I X₉₁X₉₂ILX₉₅X₉₆WIX₉₉S (SEQ ID NO: 3)

In all such embodiments:
(i) D1, D2, D3 and D4 may be in any order in the polypeptide;
(ii) amino acid linkers may be present between any of the domains, X₈ is any amino acid; X₁₇ is glutamic acid or aspartic acid; X₂i is a natural amino acid; X₃₃ is any amino acid; X₈₉ is arginine or lysine; X₉₁ is arginine or lysine; X₉₂ is arginine or lysine; X₉₅ is threonine, serine, glutamic acid, or aspartic acid; X₉₆ is aspartic acid or glutamic acid; and X₉₉ is arginine or lysine; and
(iii) wherein the polypeptide contains a total of no more than ten, no more than nine, no more than eight, no more than seven, no more than six, no more than five, no more than four, no more than three, no more than two, no more than one substitutions at positions not designated as X.

In some embodiments for polypeptides of Section A, X₈ is any amino acid. In some embodiments, X₈ is alanine, asparagine, aspartic acid, arginine, cysteine, glutamic acid, glutamine, glycine, histidine, isoleucine, leucine, lysine, methionine, phenylalanine, serine, threonine, tryptophan, tyrosine, or valine. In some embodiments, X₈ is histidine, tyrosine, or phenylalanine. In some embodiments, X₈ is tyrosine or phenylalanine. In all of these embodiments, the other variables (e.g., X₁₇, X₂₁, X₃₃, X₈₉, X₉₁, X₉₂, X₉₅, X₉₆, and X₉₉) can be as set forth in any of the embodiments described herein.

In some embodiments for polypeptides of Section A, X₃₃ is any amino acid. In some embodiments, X₃₃ is cysteine, tyrosine, lysine, glutamic acid or aspartic acid. In some embodiments, X₃₃ is cysteine, tyrosine, lysine, glutamic acid or aspartic acid. In some embodiments, X₃₃ is glutamic acid or aspartic acid. In some embodiments, X₃₃ is glutamic acid. In all of these embodiments, the other variables (e.g., X₈, X₁₇, X₂₁, X₈₉, X₉₁, X₉₂, X₉₅, X₉₆, and X₉₉) can be as set forth in any of the embodiments described herein.

In some embodiments for polypeptides of Section A, X₁₇ is aspartic acid. In some embodiments, X₁₇ is glutamic acid. In all of these embodiments, the other variables (e.g., X₈, X₂₁, X₃₃, X₈₉, X₉₁, X₉₂, X₉₅, X₉₆, and X₉₉) can be as set forth in any of the embodiments described herein.

In some embodiments for polypeptides of Section A, X₈₉ is arginine. In some embodiments, X₈₉ is lysine. In all of these embodiments, the other variables (e.g., X₈, X₁₇, X₂₁, X₃₃, X₉₁, X₉₂, X₉₅, X₉₆, and X₉₉) can be as set forth in any of the embodiments described herein.

In some embodiments for polypeptides of Section A, X₉₁ is arginine. In some embodiments, X₉₁ is lysine. In all of these embodiments, the other variables (e.g., X₈, X₁₇, X₂₁, X₃₃, X₈₉, X₉₂, X₉₅, X₉₆, and X₉₉) can be as set forth in any of the embodiments described herein.

In some embodiments for polypeptides of Section A, X₉₂ is arginine. In some embodiments, X₉₂ is lysine. In all of these embodiments, the other variables (e.g., X₈, X₁₇, X₂₁, X₃₃, X₈₉, X₉₁, X₉₅, X₉₆, and X₉₉) can be as set forth in any of the embodiments described herein.

In some embodiments for polypeptides of Section A, X₉₆ is glutamic acid. In some embodiments, X₉₆ is aspartic acid. In all of these embodiments, the other variables (e.g., X₈, X₁₇, X₂₁, X₃₃, X₈₉, X₉₁, X₉₂, X₉₅, and X₉₉) can be as set forth in any of the embodiments described herein.

In some embodiments for polypeptides of Section A, X₉₉ is arginine. In some embodiments, X₉₉ is lysine. In all of these embodiments, the other variables (e.g., X₈, X₁₇, X₂₁, X₃₃, X₈₉, X₉₁, X₉₂, X₉₅, and X₉₆) can be as set forth in any of the embodiments described herein.

In some embodiments for polypeptides of Section A, X₉₅ is threonine, serine, glutamic acid, or aspartic acid. In some embodiments, X₉₅ is threonine, glutamic acid, or aspartic acid. In some embodiments, X₉₅ is threonine or glutamic acid. In some embodiments, X₉₅ is glutamic acid. In all of these embodiments, the other variables (e.g., X₈, X₁₇, X₂₁, X₃₃, X₈₉, X₉₁, X₉₂, X₉₆, and X₉₉) can be as set forth in any of the embodiments described herein.

In some embodiments for polypeptides of Section A, X₂i is any amino acid. In som embodiments, X₂₁ is alanine, asparagine, aspartic acid, arginine, cysteine, glutamic acid, glutamine, glycine, histidine, isoleucine, leucine, lysine, methionine, proline, serine, threonine, tryptophan, tyrosine, or valine. In some embodiments, X₂₁ is lysine. In all of these embodiments, the other variables (e.g., X₈, X₁₇, X₃₃, X₈₉, X₉₁, X₉₂, X₉₅, X₉₆, and X₉₉) can be as set forth in any of the embodiments described herein.

Included herein are polypeptides wherein D1 comprises a tyrosine or phenylalanine at position 5 wherein the position numbering is according to SEQ ID NO:1 and/or D3 comprises a cysteine, aspartic acid, glutamic acid, or tyrosine at position 1 wherein the position numbering is according to SEQ ID NO:2. In all of these embodiments, the other variables (e.g., X₈, X₁₇, X₂₁, X₃₃, X₈₉, X₉₁, X₉₂, X₉₅, X₉₆, and X₉₉) can be as set forth in any of the embodiments described herein.

Included herein are polypeptides wherein D1 comprises a histidine at position 5 wherein the position numbering is according to SEQ ID NO:1 and/or D3 comprises a lysine at position 1 wherein the position numbering is according to SEQ ID NO:2. In all of these embodiments, the other variables (e.g., X₈, X₁₇, X₂₁, X₃₃, X₈₉, X₉₁, X₉₂, X₉₅, X₉₆, and X₉₉) can be as set forth in any of the embodiments described herein.

Included herein are polypeptides of Section A wherein there are 10, 9, 8, 7, 6, 5, 4, 3, 2, 1 or zero substitutions at positions not designated as X. In all of these embodiments, X₈, X₁₇, X₂₁, X₃₃, X₈₉, X₉₁, X₉₂, X₉₅, X₉₆, and X₉₉ can be as set forth in any of the embodiments described herein. Section B

Exemplary polypeptides of the present invention bind IL-2Rβ and comprise the domains D1, D2, D3, and D4 wherein:
(a) D1 comprises the amino acid sequence: KIQLFAEHAL YDAX₁₇MILKI (SEQ ID NO:21)
(b) D2 comprises an amino acid sequence at least 8 amino acids in length;
(c) D3 comprises the amino acid sequence ELEDYAFN FELILEEIAR LFESG (SEQ ID NO:2)
(d) D4 comprises the amino acid sequence EDEQEEMANKI RKILX₉₅EWIX₉₉S (SEQ ID NO:29)

Exemplary polypeptides of the present invention bind IL-2Rβ and comprise the domains D1, D2, D3, and D4 wherein:
(a) D1 comprises the amino acid sequence: KIQLYAEHAL YDAX₁₇MILKI (SEQ ID NO:32)
(b) D2 comprises an amino acid sequence at least 8 amino acids in length;
(c) D3 comprises the amino acid sequence ELEDYAFN FELILEEIAR LFESG (SEQ ID NO:2)
(d) D4 comprises the amino acid sequence EDEQEEMANRI RKILX₉₅EWIX₉₉S (SEQ ID NO:47).

Exemplary polypeptides of the present invention bind IL-2Rβ and comprise the domains D1, D2, D3, and D4 wherein:
(e) D1 comprises the amino acid sequence: KIQLFAEHAL YDAEMILKI (SEQ ID NO:27)
(f) D2 comprises an amino acid sequence at least 8 amino acids in length;
(g) D3 comprises the amino acid sequence ELEDYAFN FELILEEIAR LFESG (SEQ ID NO:2)
(h) D4 comprises the amino acid sequence EDEQEEMANKI RKILX₉₅EWIX₉₉S (SEQ ID NO:29)

Exemplary polypeptides of the present invention bind IL-2Rβ and comprise the domains D1, D2, D3, and D4 wherein:
(e) D1 comprises the amino acid sequence: KIQLYAEHAL YDAEMILKI (SEQ ID NO:44)
(f) D2 comprises an amino acid sequence at least 8 amino acids in length;
(g) D3 comprises the amino acid sequence ELEDYAFN FELILEEIAR LFESG (SEQ ID NO:2)
(h) D4 comprises the amino acid sequence EDEQEEMANRI RKILX₉₅EWIX₉₉S (SEQ ID NO:47).

Exemplary polypeptides of the present invention bind IL-2Rβ and comprise the domains D1, D2, D3, and D4 wherein:
(a) D1 comprises the amino acid sequence: KIQLYAEHAX₁₃ YDAX₁₇MILNI (SEQ ID NO:20)
(b) D2 comprises an amino acid sequence at least 8 amino acids in length;
(c) D3 comprises the amino acid sequence ELEDYAFN FELILEEIAR LFESG (SEQ ID NO:2)
(d) D4 comprises the amino acid sequence EDEQEEMANAI IT ILX₉₅SWIX₉₉S (SEQ ID NO:22)

In all such embodiments for polypeptides in Section B:
(i) D1, D2, D3 and D4 may be in any order in the polypeptide;
(ii) amino acid linkers may be present between any of the domains,

X₁₃, if present, is arginine or lysine; X₁₇, if present, is glutamic acid or aspartic acid; X₉₅ is threonine, serine, glutamic acid, or aspartic acid; and X₉₉ is arginine or lysine;
(iii) wherein the polypeptide contains a total of no more than ten, no more than nine, no more than eight, no more than seven, no more than six, no more than five, no more than four, no more than three, no more than two, or no more than one substitution at positions not designated as X.

In some embodiments for polypeptides of Section B, X₁₇ is aspartic acid. In some embodiments, X₁₇ is glutamic acid. In all of these embodiments, the other variables (e.g., X₁₃, X₉₅, and X₉₉) can be as set forth in any of the embodiments described herein.

In some embodiments for polypeptides of Section B, X₉₉ is arginine. In some embodiments, X₉₉ is lysine. In all of these embodiments, the other variables (e.g., X₁₃, X₉₅, and X₁₇) can be as set forth in any of the embodiments described herein.

In some embodiments for polypeptides of Section B, X₉₅ is threonine, serine, glutamic acid, or aspartic acid. In some embodiments, X₉₅ is threonine, glutamic acid, or aspartic acid. In some embodiments, X₉₅ is threonine or glutamic acid. In some embodiments, X₉₅ is glutamic acid. In all of these embodiments, the other variables (e.g., X₁₃, X₁₇, and X₉₉) can be as set forth in any of the embodiments described herein.

Included herein for polypeptides of Section B are polypeptides wherein one, two, three, or four of the following is true: if there is a substitution at position 10 of D4, it is to arginine or lysine; if there is a substitution at position 12 of D4 it is to lysine; if there is a substitution at position 13 of D4, it is to arginine; and/or if there is a substitution at position 17 of D4, it is to aspartic acid, wherein the position numbering of D4 is according to SEQ ID NO: 29 or 47. In all of these embodiments, the other variables (e.g., X₁₃, X₁₇, X₉₅, and X₉₉) can be as set forth in any of the embodiments described herein.

Included herein for polypeptides of Section B are polypeptides wherein if there is a substitution at position 12 of D4 it is to lysine; if there is a substitution at position 13 of D4, it is to arginine; and/or if there is a substitution at position 17 of D4, it is to aspartic acid, wherein the position numbering of D4 is according to SEQ ID NO: 29 or 47. In all of these embodiments, the other variables (e.g., X₁₃, X₁₇, X₉₅, and X₉₉) can be as set forth in any of the embodiments described herein.

In some embodiments for polypeptides noted above in Section B, there are no substitutions at positions 12, 13, and 17 of D4 wherein the position numbering of D4 is according to SEQ ID NO: 29 or 47. In some embodiments for polypeptides noted above in Section B, there are no substitutions at position 10 of D4 wherein the position numbering of D4 is according to SEQ ID NO: 29 or 47. In all of these embodiments, the other variables (e.g., X₁₃, X₁₇, X₉₅, and X₉₉) can be as set forth in any of the embodiments described herein.

In some embodiments for polypeptides noted above in Section B, if there is a substitution at the glutamic acid of position 14 of D1, it is to aspartic acid, wherein the position numbering of D1 is according to SEQ ID NO: 27 or 44. In some embodiments for polypeptides noted above in Section B, there are no substitutions at position 14 of D1. In all of these embodiments, X₁₃, X₉₅, and X₉₉ can be as set forth in any of the embodiments described herein.

Included herein are polypeptides of Section B wherein there are 10, 9, 8, 7, 6, 5, 4, 3, 2, 1 or zero substitutions at positions not designated as X. In all of these embodiments, the other variables (e.g., X₁₃, X₁₇, X₉₅, and X₉₉) can be as set forth in any of the embodiments described herein.

Included herein are polypeptides of Section B wherein X₁₃ is arginine, X₁₇ is glutamic acid, and X₉₉ is arginine.

### Section C

Exemplary polypeptides of the present invention bind IL-2Rβ and comprise the domains D1, D2, D3, and D4 wherein:
(a) D1 comprises an amino acid sequence at least 80% identical to the amino acid sequence: KIQLFAEHAL YDAEMILKI (SEQ ID NO: 27)
(b) D2 comprises an amino acid sequence at least 8 amino acids in length;
(c) D3 comprises an amino acid sequence at least 80% identical to the amino acid sequence ELEDYAFN FELILEEIAR LFESG (SEQ ID NO: 2)
(d) D4 comprises an amino acid sequence at least 80% identical to the amino acid sequence EDEQEEMANKI RKILEEWIRS (SEQ ID NO: 43) wherein D1, D2, D3 and D4 may be in any order in the polypeptide; amino acid linkers may be present between any of the domains; and wherein the polypeptide comprises a threonine, serine, glutamic acid or aspartic acid at position 16 of D4, and an arginine or lysine at position 20 of D4, wherein the position numbering of D4 is according to SEQ ID NO: 43. In some such aspects, the polypeptide comprises a glutamic acid or aspartic acid at position 14 of D1 wherein the position numbering of D1 is according to SEQ ID NO:27.

Included herein are polypeptides noted above in Section C wherein one two, three, or four of the following are true: there is an arginine or lysine at position 10 of D4, there is a lysine or arginine at position 12 of D4, there is an arginine or lysine at position 13 of D4, and there is a glutamic acid or aspartic acid at position 17 of D4.

Included herein are polypeptides noted above in Section C that comprise an arginine or lysine at position 10 of D4, an arginine at position 12 of D4, a lysine at position 13 of D4, and a glutamic acid at position 17 of D4.

Included herein are polypeptides noted above in Section C that comprise a glutamic acid at position 14 of D1 and an arginine at position 20 of D4. In any of the embodiments noted herein for polypeptides of Section C, there can be a glutamic acid at position 16 of D4.

Included herein are polypeptides noted above in Section C wherein D1 comprises an amino acid sequence at least 85%, at least 90%, at least 95% or 100% identical to the amino acid sequence set forth in SEQ ID NO:27; D3 comprises an amino acid sequence at least 85%, at least 90%, at least 95% , or 100% identical to the amino acid sequence set forth in SEQ ID NO:2 and D4 comprises an amino acid sequence at least at least 85%, 90%, at least 95% , or 100% identical to the amino acid sequence set forth in SEQ ID NO:43.

Included herein are polypeptides noted above in Section C wherein D3 comprises an amino acid sequence at least 90%, at least 95%, or 100% identical to the amino acid sequence set forth in SEQ ID NO:2 and D4 comprises an amino acid sequence at least 90%, at least 95%, or 100% identical to the amino acid sequence set forth in SEQ ID NO:43.

Domain D2 for the polypeptides of Section A, B, and C is at least 8 amino acid in length. Included in the present invention are polypeptides of Section A, B, and C wherein D2 is at least 19 amino acids in length. Included in the present invention are polypeptides of Section A, B, and C wherein D2 comprises an amino acid sequence at least 84%, 89%, or 94% identical to the amino acid sequence KDEAEK AKRMKEWMKR IKT (SEQ ID NO: 18) or when D2 comprises the amino acid sequence of SEQ ID NO:18. Included in the present invention are polypeptides of Section A, B, and C wherein the order of the four domains is D1-D3-D2-D4.

### Section D

Exemplary polypeptides of the present invention bind IL-2Rβ and comprise an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of SEQ ID NO: 7:

(SEQ ID NO: 7)
KIQLYAEHAL YDAEMILKIV KTNSPPAEEE LEDYAFNFEL
ILEEIARLFE SGDQKDEAEK AKRMKEWMKR IKTTASEDEQ
EEMANRIRKI LEEWIRS;

wherein the polypeptide comprises:
a glutamic acid, aspartic acid, threonine, or serine at position 95,
an arginine or lysine at position 99, and
one or more of, two or more of, three or more of, four or more of, or all five of:
a glutamic acid or aspartic acid at position 17;
an arginine or lysine at position 89
an arginine or lysine at position 91,
a lysine or arginine at position 92, and/or
a glutamic acid or aspartic acid at position 96
wherein the position numbering is according to SEQ ID NO: 7, provided that the lysine
at the N terminus of SEQ ID NO: 7 is designated as position 4.

Exemplary polypeptides of the present invention bind IL-2Rβ and comprise an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of SEQ ID NO: 8:

(SEQ ID NO: 8)
KIQLFAEHAL YDAEMILKIV KTNSPPAEEE LEDYAFNFEL
ILEEIARLFE SGDQKDEAEK AKRMKEWMKR IKTTASEDEQ
EEMANKIRKI LEEWIRS;

wherein the polypeptide comprises:
a glutamic acid, aspartic acid, threonine, or serine at position 95,
an arginine or lysine at position 99, and
one or more of, two or more of, three or more of, four or more of, or all five of:
a glutamic acid or aspartic acid at position 17;
an arginine or lysine at position 89
an arginine or lysine at position 91,
a lysine or arginine at position 92, and/or
a glutamic acid or aspartic acid at position 96
wherein the position numbering is according to SEQ ID NO: 8, provided that the lysine at the N terminus of SEQ ID NO: 8 is designated as position 4.

Exemplary polypeptides of the present invention bind IL-2Rβ and comprise an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of SEQ ID NO: 13:

(SEQ ID NO: 13)
KIQLYAEHAR YDAEMILNIV KTNSPPAEEE LEDYAFNFEL
ILEEIARLFE SGDQKDEAEK AKRMKEWMKR IKTTASEDEQ
EEMANAIITI LTSWIRS;

wherein the polypeptide comprises:
a glutamic acid or aspartic acid at position 17, a glutamic acid, aspartic acid, threonine, or serine at position 95, an arginine or lysine at position 99, and
wherein the position numbering is according to SEQ ID NO: 13, provided that the lysine at the N terminus of SEQ ID NO: 13 is designated as position 4.

In some embodiments for polypeptides of Section D, there is an arginine at position 99. In other embodiments, there is a lysine at position 99. In such embodiments, positions 17, 95, and 13 can be as set forth in any of the embodiments described herein.

In some embodiments for polypeptides of Section D, there is a glutamic acid at position 17. In some embodiments, there is an aspartic acid at position 17.

In some embodiments for polypeptides of Section D, there is a glutamic acid, aspartic acid, threonine, or serine at position 95. In some embodiments, there is a glutamic acid or threonine at position 95. In some embodiments, there is a glutamic acid at position 95. In other embodiments, there is a threonine at position 95.

In some embodiments for polypeptides of Section D, there is a leucine, isoleucine, valine, arginine, or lysine at position 13. In some embodiments, there is a leucine or arginine at position 13. In some embodiments, there is a leucine at position 13. In some embodiments, there is an arginine at position 13.

Included in the present invention are the above noted polypeptides of Section D wherein the polypeptide comprises a glutamic acid or aspartic acid at position 17, a glutamic acid, aspartic acid, threonine or serine at position 95, and an arginine or lysine at position 99.

Included in the present invention are the above noted polypeptides of Section D wherein the polypeptide comprises a glutamic acid at position 17, a glutamic acid aspartic acid, threonine or serine at position 95, and an arginine at position 99.

Included in the present invention are the above noted polypeptides of Section D wherein the polypeptide comprises a glutamic acid at position 17, a glutamic acid or threonine at position 95, and an arginine at position 99.

Included in the present invention are the above noted polypeptides of Section D wherein the polypeptide comprises one, two, three or four of an arginine or lysine at position 89, an arginine or lysine at position 91, an arginine or lysine at position 92, and a glutamic acid or aspartic acid at position 96.

Included in the present invention are the above noted polypeptides of Section D wherein the polypeptide comprises an arginine or lysine at position 91, an arginine or lysine at position 92, and a glutamic acid or aspartic acid at position 96.

Included in the present invention are any of the above noted polypeptides of Section D wherein the polypeptide comprises one, two, three or four of: an arginine or lysine at position 89, an arginine at position 91, a lysine at position 92, and a glutamic acid at position 96.

Included in the present invention are any of the above noted polypeptides of Section D wherein the polypeptide comprises an arginine at position 91, a lysine at position 92, and a glutamic acid at position 96.

Included in the present invention are any of the above noted polypeptides of Section D wherein the polypeptide comprises any amino acid at position 21.

Included in the present invention are any of the above noted polypeptides of Section D wherein the polypeptide comprises an alanine, asparagine, aspartic acid, arginine, cysteine, glutamic acid, glutamine, glycine, histidine, isoleucine, leucine, lysine, methionine, proline, serine, threonine, tryptophan, tyrosine, or valine at position 21.

Included in the present invention are any of the above noted polypeptides of Section D wherein the polypeptide comprises a lysine or arginine at position 21.

Included in the present invention are any of the above noted polypeptides of Section D wherein the polypeptide comprises a histidine, tyrosine or phenylalanine at position 8.

Included in the present invention are any of the above noted polypeptides of Section D wherein the polypeptide comprises a tyrosine or phenylalanine at position 8.

Included in the present invention are any of the above noted polypeptides of Section D wherein the polypeptide comprises a cysteine, tyrosine, lysine, glutamic acid or aspartic acid at position 33.

Included in the present invention are any of the above noted polypeptides of Section D wherein the polypeptide comprises a glutamic acid or aspartic acid at position 33.

Included in the present invention are any of the above noted polypeptides of Section D wherein the polypeptide comprises a tyrosine or phenylalanine at position 8 and/or a glutamic acid or aspartic acid at position 33.

Included in the present invention are any of the above noted polypeptides comprising at least 3 amino acid N terminal to the lysine and the amino acids are proline-lysine-lysine-.

### Section E

Included in the present invention are polypeptides that bind IL-2Rβ and comprise the amino acid sequence of SEQ ID NO: 17, 18, or 19:

(SEQ ID NO: 17)
KIQLYAEHAL YDAX₁₇MILKIV KTNSPPAEEE LEDYAFNFEL
ILEEIARLFE SGDQKDEAEK AKRMKEWMKR IKTTASEDEQE
EMANX₈₉IX₉₁X₉₂I LX₉₅X₉₆WIX₉₉S
(SEQ ID NO: 18)
KIQLFAEHAL YDAX₁₇MILKIV KTNSPPAEEE LEDYAFNFEL
ILEEIARLFE SGDQKDEAEK AKRMKEWMKR IKTTASEDEQE
EMANX₈₉IX₉₁X₉₂I LX₉₅X₉₆WIX₉₉S
(SEQ ID NO: 19)
KIQLX₈AEHAL YDAX₁₇MILX₂₁IV KTNSPPAEEX₃₃ LEDYAFNFEL
ILEEIARLEE SGDQKDEAEK AKRMKEWMKR IKTTASEDEQE
EMANX₈₉IX₉₁X₉₂I LX₉₅X₉₆WIX₉₉S

wherein:
X₈ is any amino acid;
X₁₇ is glutamic acid or aspartic acid;
X₂i is any amino acid;
X₃₃ is any amino acid;
X₈₉ is arginine or lysine;
X₉₁ is arginine or lysine;
X₉₂ is arginine or lysine;
X₉₅ is threonine, serine, glutamic acid, or aspartic acid;
X₉₆ is aspartic acid or glutamic acid; and
X₉₉ is arginine or lysine
wherein the polypeptide contains a total of no more than ten, no more than nine, no more than eight, no more than seven, no more than six, no more than five, no more than four, no more than three substitutions, or no more than two substitutions, additions, and/or deletions at amino acid positions in SEQ ID NO: 17, 18 or 19 not designated as X.

Included in the present invention are any of the above noted polypeptides of Section E wherein the polypeptide comprises one, two, or three of: (i) an asparagine or lysine at position 21, (ii) a cysteine, tyrosine, glutamic acid, lysine, or aspartic acid at position 33 and/or (iii) a tyrosine, histidine, or phenylalanine at position 8; or any combination thereof, wherein the position numbering is according to SEQ ID NO:17 or 18, provided that the lysine at the N terminus of SEQ ID NO: 17 and 18 is designated as position 4.

Included in the present invention are any of the above noted polypeptides of Section E wherein the polypeptide comprises one, two, or three of: (i) an asparagine or lysine at position 21, (ii) a cysteine, tyrosine, glutamic acid or aspartic acid at position 33 and/or (iii) a tyrosine, or phenylalanine at position 8; or any combination thereof, wherein the position numbering is according to SEQ ID NO:17 or 18, provided that the lysine at the N terminus of SEQ ID NO: 17 and 18 is designated as position 4.

In some embodiments for polypeptides of Section E, X₈ is any amino acid. In some embodiments, X₈ is alanine, asparagine, aspartic acid, arginine, cysteine, glutamic acid, glutamine, glycie, histidine, isoleucine, leucine, lysine, methionine, phenylalanine, serine, threonine, tryptophan, tyrosine, or valine. In some embodiments, X₈ is histidine, tyrosine, or phenylalanine. In some embodiments, X₈ is tyrosine or phenylalanine. In all of these embodiments, the other variables (e.g., X₁₇, X₂₁, X₃₃, X₈₉, X₉₁, X₉₂, X₉₅, X₉₆, and X₉₉) can be as set forth in any of the embodiments described herein.

In some embodiments for polypeptides of Section E, X₃₃ is any amino acid. In some embodiments, X₃₃ is cysteine, tyrosine, lysine, glutamic acid or aspartic acid. In some embodiments, X₃₃ is cysteine, tyrosine, lysine, glutamic acid or aspartic acid. In some embodiments, X₃₃ is glutamic acid or aspartic acid. In some embodiments, X₃₃ is glutamic acid. In all of these embodiments, the other variables (e.g., X₈, X₁₇, X₂₁, X₈₉, X₉₁, X₉₂, X₉₅, X₉₆, and X₉₉) can be as set forth in any of the embodiments described herein.

In some embodiments for polypeptides of Section E, X₁₇ is glutamic acid or aspartic acid. In some embodiments, X₁₇ is glutamic acid. In all of these embodiments, the other variables (e.g., X₈, X₂₁, X₃₃, X₈₉, X₉₁, X₉₂, X₉₅, X₉₆, and X₉₉) can be as set forth in any of the embodiments described herein.

In some embodiments for polypeptides of Section E, X₈₉ is arginine. In some embodiments, X₈₉ is lysine. In all of these embodiments, the other variables (e.g., X₈, X₁₇, X₂₁, X₃₃, X₉₁, X₉₂, X₉₅, X₉₆, and X₉₉) can be as set forth in any of the embodiments described herein.

In some embodiments for polypeptides of Section E, X₉₁ is arginine. In some embodiments, X₉₁ is lysine. In all of these embodiments, the other variables (e.g., X₈, X₁₇, X₂₁, X₃₃, X₈₉, X₉₂, X₉₅, X₉₆, and X₉₉) can be as set forth in any of the embodiments described herein.

In some embodiments for polypeptides of Section E, X₉₂ is arginine. In some embodiments, X₉₂ is lysine. In all of these embodiments, the other variables (e.g., X₈, X₁₇, X₂₁, X₃₃, X₈₉, X₉₁, X₉₅, X₉₆, and X₉₉) can be as set forth in any of the embodiments described herein.

In some embodiments for polypeptides of Section E, X₉₆ is glutamic acid. In some embodiments, X₉₆ is aspartic acid. In all of these embodiments, the other variables (e.g., X₈, X₁₇, X₂₁, X₃₃, X₈₉, X₉₁, X₉₂, X₉₅, and X₉₉) can be as set forth in any of the embodiments described herein.

In some embodiments for polypeptides of Section E, X₉₉ is arginine. In some embodiments, X₉₉ is lysine. In all of these embodiments, the other variables (e.g., X₈, X₁₇, X₂₁, X₃₃, X₈₉, X₉₁, X₉₂, X₉₅, and X₉₆) can be as set forth in any of the embodiments described herein.

In some embodiments for polypeptides of Section E, X₉₅ is threonine, serine, glutamic acid, or aspartic acid. In some embodiments, X₉₅ is threonine, glutamic acid, or aspartic acid. In some embodiments, X₉₅ is threonine or glutamic acid. In some embodiments, X₉₅ is glutamic acid. In all of these embodiments, the other variables (e.g., X₈, X₁₇, X₂₁, X₃₃, X₈₉, X₉₁, X₉₂, X₉₆, and X₉₉) can be as set forth in any of the embodiments described herein.

In some embodiments for polypeptides of Section E, X₂₁ is any amino acid. In som embodiments, X₂₁ is alanine, asparagine, aspartic acid, arginine, cysteine, glutamic acid, glutamine, glycie, histidine, isoleucine, leucine, lysine, methionine, proline, serine, threonine, tryptophan, tyrosine, or valine. In some embodiments, X₂₁ is lysine. In all of these embodiments, the other variables (e.g., X₈, X₁₇, X₃₃, X₈₉, X₉₁, X₉₂, X₉₅, X₉₆, and X₉₉) can be as set forth in any of the embodiments described herein.

Included herein are polypeptides of Section E wherein there are 10, 9, 8, 7, 6, 5, 4, 3, 2, 1 or zero substitutions at positions not designated as X.

Included in the present invention are polypeptides comprising an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or 100% identical to the amino acid sequence of SEQ ID NO: 9-16 and 37-38:

(SEQ ID NO: 9)
PKKKIQLYAE HALYDAEMIL KIVKTNSPPA EEELEDYAFN
FELILEEIAR LFESGDQKDE AEKAKRMKEW MKRIKTTASE
DEQEEMANRI RKILEEWIRS.
(SEQ ID NO: 10)
PKKKIQLFAE HALYDAEMIL KIVKTNSPPA EEELEDYAFN
FELILEEIAR LFESGDQKDE AEKAKRMKEW MKRIKTTASE
DEQEEMANKI RKILEEWIRS.
(SEQ ID NO: 11)
KIQLHAEHAR YDAEMILNIV KTNSPPAEEK LEDYAFNFEL
ILEEIARLFE SGDQKDEAEK AKRMKEWMKR IKTTASEDEQ
EEMANAIITI LTSWIRS;
(SEQ ID NO: 12)
KIQLHAEHAL YDAEMILKIV KTNSPPAEEK LEDYAFNFEL
ILEEIARLFE SGDQKDEAEK AKRMKEWMKR IKTTASEDEQ
EEMANRIRKI LEEWIRS;
(SEQ ID NO: 13)
KIQLYAEHAR YDAEMILNIV KTNSPPAEEE LEDYAFNFEL
ILEEIARLFE SGDQKDEAEK AKRMKEWMKR IKTTASEDEQ
EEMANAIITI LTSWIRS.
(SEQ ID NO: 14)
PKKKIQLHAE HARYDAEMIL NIVKTNSPPA EEKLEDYAFN
FELILEEIAR LFESGDQKDE AEKAKRMKEW MKRIKTTASE
DEQEEMANAI ITILTSWIRS;
(SEQ ID NO: 15)
PKKKIQLHAE HALYDAEMIL KIVKTNSPPA EEKLEDYAFN
FELILEEIAR LFESGDQKDE AEKAKRMKEW MKRIKTTASE
DEQEEMANRI RKILEEWIRS;
(SEQ ID NO: 16)
PKKKIQLYAE HARYDAEMIL NIVKTNSPPA EEELEDYAFN
FELILEEIAR LFESGDQKDE AEKAKRMKEW MKRIKTTASE
DEQEEMANAI ITILTSWIRS
(SEQ ID NO: 37)
KIQLHAE HALYDAEMIL KIVKTNSPPA EEKLEDYAFN
FELILEEIAR LFESGDQKDE AEKAKRMKEW MKRIKTTASE
DEQEEMANKI RKILEEWIRS;
(SEQ ID NO: 39)
PKKKIQLHAE HALYDAEMIL KIVKTNSPPA EEKLEDYAFN
FELILEEIAR LFESGDQKDE AEKAKRMKEW MKRIKTTASE
DEQEEMANKI RKILEEWIRS.

### Section F

Included in the present invention are polypeptides that bind IL-2Rβ and comprise the amino acid sequence of SEQ ID NO: 33 or 36:

(SEQ ID NO: 33)
KIQLYAEHAL YDAX₁₇MILKIV KTNSPPAEEE LEDYAFNFEL
ILEEIARLFE SGDQKDEAEK AKRMKEWMKR IKTTASEDEQE
EMANRIRKI LX₉₅EWIX₉₉S
(SEQ ID NO: 36)
KIQLFAEHAL YDAX₁₇MILKIV KTNSPPAEEE LEDYAFNFEL
ILEEIARLFE SGDQKDEAEK AKRMKEWMKR IKTTASEDEQE
EMANKIRKI LX₉₅EWIX₉₉S

wherein:
   X₁₇ is glutamic acid or aspartic acid;
   X₉₅ is threonine, serine, glutamic acid, or aspartic acid; and
   X₉₉ is arginine or lysine
wherein the polypeptide contains a total of no more than ten, no more than nine, no more than eight, no more than seven, no more than six, no more than five, no more than four, no more than three substitutions, or no more than two substitutions, additions, and/or deletions at amino acid positions in SEQ ID NO: 33, or 36 not designated as X.

Included in the present invention are any of the above noted polypeptides of Section E wherein one, two, three, or four of the following are true: if there is a substitution at position 89, it is to arginine or lysine; if there is a substitution at position 91, it is to lysine; if there is a substitution at position 92, it is to arginine; and/or if there is a substitution at position 96, it is to aspartic acid, wherein the position numbering is according to SEQ ID NO: 33 or SEQ ID NO:36, provided that the lysine at the N terminus of SEQ ID NO: 33 and SEQ ID NO:36 is designated as position 4.

Included in the present invention are any of the above noted polypeptides of Section E wherein if there is a substitution at position 91, it is to lysine; if there is a substitution at position 92, it is to arginine; and/or if there is a substitution at position 96, it is to aspartic acid, wherein the position numbering is according to SEQ ID NO: 33 or SEQ ID NO:36, provided that the lysine at the N terminus of SEQ ID NO: 33 and SEQ ID NO:36 is designated as position 4.

Included in the present invention are polypeptides that bind IL-2Rβ and comprise the amino acid sequence of SEQ ID NO: 45 or 46:

(SEQ ID NO: 45)
KIQLYAEHAL YDAEMILKIV KTNSPPAEEE LEDYAFNFEL
ILEEIARLFE SGDQKDEAEK AKRMKEWMKR IKTTASEDEQE
EMANRIRKI LX₉₅EWIX₉₉S
(SEQ ID NO: 46)
KIQLFAEHAL YDAEMILKIV KTNSPPAEEE LEDYAFNFEL
ILEEIARLFE SGDQKDEAEK AKRMKEWMKR IKTTASEDEQE
EMANKIRKI LX₉₅EWIX₉₉S

wherein:
   X₉₅ is threonine, serine, glutamic acid, or aspartic acid; and
   X₉₉ is arginine or lysine
wherein the polypeptide contains a total of no more than ten, no more than nine, no more than eight, no more than seven, no more than six, no more than five, no more than four, no more than three substitutions, or no more than two substitutions, additions, and/or deletions at amino acid positions in SEQ ID NO: 45, or 46 not designated as X.

Included in the present invention are any of the above noted polypeptides wherein one, two, three, or four of the following are true: if there is a substitution at position 17, it is to aspartic acid, if there is a substitution at position 89, it is to arginine or lysine; if there is a substitution at position 91, it is to lysine; if there is a substitution at position 92, it is to arginine; and/or if there is a substitution at position 96, it is to aspartic acid, wherein the position numbering is according to SEQ ID NO: 45 or SEQ ID NO:46, provided that the lysine at the N terminus of SEQ ID NO: 45 and SEQ ID NO:46 is designated as position 4.

Included in the present invention are any of the above noted polypeptides wherein if there is a substitution at position 17, it is to aspartic acid; if there is a substitution at position 91, it is to lysine; if there is a substitution at position 92, it is to arginine; and/or if there is a substitution at position 96, it is to aspartic acid, wherein the position numbering is according to SEQ ID NO: 45 or SEQ ID NO:46, provided that the lysine at the N terminus of SEQ ID NO: 45 and SEQ ID NO:46 is designated as position 4.

Included in the present invention are any of the above noted polypeptides of Section F wherein there are no substitutions at positions 91, 92, and 96 wherein the position numbering is according to SEQ ID NO: 33, SEQ ID NO:36, SEQ ID NO: 45, or SEQ ID NO:46, provided that the lysine at the N terminus of SEQ ID NO: 33, SEQ ID NO:36, SEQ ID NO: 45, or SEQ ID NO:46 is designated as position 4.

Included in the present invention are any of the above noted polypeptides of Section F wherein there are no substitutions at position 89 wherein the position numbering is according to SEQ ID NO: 33, SEQ ID NO:36, SEQ ID NO: 45, or SEQ ID NO:46, provided that the lysine at the N terminus of SEQ ID NO: 33, SEQ ID NO:36, SEQ ID NO: 45, or SEQ ID NO:46 is designated as position 4.

Included in the present invention are any of the above noted polypeptides of Section F wherein: if there is a substitution at the tyrosine of position 8, it is a substitution to phenylalanine, wherein the position numbering is according to SEQ ID NO: 33, SEQ ID NO:36, SEQ ID NO: 45, or SEQ ID NO:46, provided that the lysine at the N terminus of SEQ ID NO: 33, SEQ ID NO:36, SEQ ID NO: 45, or SEQ ID NO:46 is designated as position 4.

Included in the present invention are any of the above noted polypeptides of Section F wherein: if there is a substitution at the tyrosine of position 8, it is a substitution to histidine, wherein the position numbering is according to SEQ ID NO: 33, SEQ ID NO:36, SEQ ID NO: 45, or SEQ ID NO:46, provided that the lysine at the N terminus of SEQ ID NO: 33, SEQ ID NO:36, SEQ ID NO: 45, or SEQ ID NO:46 is designated as position 4.

Included in the present invention are any of the above noted polypeptides of Section F wherein: if there is a substitution at position 33, it is a substitution to cysteine, aspartic acid, or tyrosine, wherein the position numbering is according to SEQ ID NO: 33, SEQ ID NO:36, SEQ ID NO: 45, or SEQ ID NO:46, provided that the lysine at the N terminus of SEQ ID NO: 33, SEQ ID NO:36, SEQ ID NO: 45, or SEQ ID NO:46 is designated as position 4.

Included in the present invention are any of the above noted polypeptides of Section F wherein: if there is a substitution at position 33, it is a substitution to lysine, wherein the position numbering is according to SEQ ID NO: 33, SEQ ID NO:36, SEQ ID NO: 45, or SEQ ID NO:46, provided that the lysine at the N terminus of SEQ ID NO: 33, SEQ ID NO:36, SEQ ID NO: 45, or SEQ ID NO:46 is designated as position 4.

Included in the present invention are any of the above noted polypeptides of Section F wherein X₁₇ is glutamic acid and X₉₉ is arginine.

Included in the present invention are any of the above noted polypeptides of Section F wherein X₉₅ is threonine, glutamic acid, or aspartic acid; X₉₅ is threonine, or X₉₅ is glutamic acid.

### Additional Exemplary Polypeptides of the Present Invention:

In a first embodiment, the present invention includes a non-naturally occurring interleukin-2 receptor β (IL-2Rβ) binding protein that has reduced binding affinity to the human IL-2 gamma receptor as compared to IL-2 and (i) comprises an amino acid sequence at set forth in SEQ ID NO:28:
X¹X²X³KIQLX^{8A}AEHAX^{13A}YDAX^{17A}MILX^{21A}IJ¹X³³ALEDYAFNFELILEEIARLFESGJ²Z¹J³ EDEQEEMANX^{89A}IX^{91A}X^{92A}ILX^{95A}X^{96A}WIX^{99A}X¹⁰⁰ (SEQ ID NO:28) wherein (a) X¹, X², X³ and X¹⁰⁰ are, independently, any amino acid and can be present or absent; (b) J¹, J² and J³ are, independently, amino acid linkers that can be present or absent; (c) Z¹ is an amino acid helical domain of at least 8 amino acids in length; (d) X^{13A} is R, X^{17A} is E, X^{21A} is N, X^{89A} is A, X^{91A} is X^{92A} is T, X^{95A} is E or T or S, X^{96A} is S, and X^{99A} is R; and (e) X^{8A} is Y or H, and X^{33A} is E or K.

In a second embodiment, the present invention includes a non-naturally occurring interleukin-2 receptor β (IL-2Rβ) binding protein that has reduced binding affinity to the human IL-2 gamma receptor as compared to IL-2 and (i) comprises an amino acid sequence at set forth in SEQ ID NO:28 wherein (a) X¹, X², X³ and X¹⁰⁰ are, independently, any amino acid and can be present or absent; (b) J¹, J² and J³ are, independently, amino acid linkers that can be present or absent; (c) Z¹ is an amino acid helical domain of at least 8 amino acids in length; (d) X^{13A} is L, X^{17A} is E, X^{21A} is K, X^{89A} is R, X^{91A} is R, X^{92A} is K, X^{95A} is E or T or S, X^{96A} is E, and X^{99A} is R; and (e) X^{8A} is Y or H; and X^{33A} is E or K.

In a third embodiment, the present invention includes a non-naturally occurring interleukin-2 receptor β (IL-2Rβ) binding protein that has reduced binding affinity to the human IL-2 gamma receptor as compared to IL-2 and (i) comprises an amino acid sequence at least 80%, at least 85%, at least 90% or at least 95% identical to the amino acid sequence set forth in SEQ ID NO:28 wherein (a) X¹, X², X³ and X¹⁰⁰ of SEQ ID NO:28 are, independently, any amino acid and can be present or absent; (b) J¹, J² and J³ of SEQ ID NO:28 are, independently, amino acid linkers that can be present or absent; (c) Z¹ of SEQ ID NO:28 is an amino acid helical domain of at least 8 amino acids in length; (d) X^{13A} of SEQ ID NO:28 is R, X^{17A} of SEQ ID NO:28 is E, X^{21A} of SEQ ID NO:28 is N, X^{89A} of SEQ ID NO:28 is A, X^{91A} of SEQ ID NO:28 is I, X^{92A} of SEQ ID NO:28 is T, X^{95A} of SEQ ID NO:28 is E or T or S, X^{96A} of SEQ ID NO:28 is S, and X^{99A} of SEQ ID NO:28 is R; and (e) X^{8A} of SEQ ID NO:28 is Y or H, and X^{33A} of SEQ ID NO:28 is E or K; provided that, in the resultant protein, X^{17A} is glutamic acid, X^{95A} is glutamic acid, threonine or serine and X^{99A} is arginine.

In a fourth embodiment, the present invention includes a non-naturally occurring interleukin-2 receptor β (IL-2Rβ) binding protein that has reduced binding affinity to the human IL-2 gamma receptor as compared to IL-2 and (i) comprises an amino acid sequence at least 80%%, at least 85%, at least 90% or at least 95% identical to the amino acid sequence set forth in SEQ ID NO:28 wherein (a) X¹, X², X³ and X¹⁰⁰ of SEQ ID NO:28 are, independently, any amino acid and can be present or absent; (b) J¹, J² and J³ of SEQ ID NO:28 are, independently, amino acid linkers that can be present or absent; (c) Z₁ of SEQ ID NO:28 is an amino acid helical domain of at least 8 amino acids in length; (d) X^{13A} of SEQ ID NO:28 is L, X^{17A} of SEQ ID NO:28 is E, X^{21A} of SEQ ID NO:28 is K, X^{89A} of SEQ ID NO:28 is R, X^{91A} of SEQ ID NO:28 is R, X^{92A} of SEQ ID NO:28 is K, X^{95A} of SEQ ID NO:28 is E or T or S, X^{96A} of SEQ ID NO:28 is E, and X^{99A} of SEQ ID NO:28 is R; and (e) X^{8A} of SEQ ID NO:28 is Y or H, and X^{33A} of SEQ ID NO:28 is E or K; provided that, in the resultant protein, X^{17A} is glutamic acid, X^{95A} is glutamic acid, threonine or serine and X⁹⁹A is arginine.

In a fifth embodiment, the present invention includes any one of the IL-2Rβ binding proteins of embodiment 3, wherein, in the resultant protein, X^{13A} is arginine.

In a sixth embodiment, the present invention includes any one of the IL-2Rβ binding proteins of embodiments 3 or 5, wherein, in the resultant protein, X^{95A} is threonine.

In a seventh embodiment, the present invention includes any one of the IL-2Rβ binding proteins of embodiment 4, wherein, in the resultant protein, one or more of the following is true: X^{21A} is lysine, X^{89A} is arginine, X^{91A} is arginine, X^{92A} is lysine, and X⁹⁶A is glutamic acid.

In an eighth embodiment, the present invention includes any one of the IL-2Rβ binding proteins of embodiment 7, wherein, in the resultant protein, X^{89A} is arginine, X⁹¹A is arginine, X^{92A} is lysine, and X^{96A} is glutamic acid.

In a ninth embodiment, the present invention includes any one of the IL-2Rβ binding proteins of embodiment 8, wherein in the resultant protein, X^{21A} is lysine.

In a tenth embodiment, the present invention includes any one of the IL-2Rβ binding proteins of embodiment 4, 7, 8, and 9, wherein in the resultant protein, X^{95A} is glutamic acid.

In an eleventh embodiment, the present invention includes any one of the IL-2Rβ binding proteins of embodiments 1-10, wherein X¹, X² and X³ are present.

In a twelfth embodiment, the present invention includes any one of the IL-2Rβ binding proteins of embodiment 11, wherein X¹, X² and X³ are PKK, respectively.

In a thirteenth embodiment, the present invention includes any one of the IL-2Rβ binding proteins of embodiments 1-10, wherein X¹, X² and X³ are absent.

In a fourteenth embodiment, the present invention includes any one of the IL-2Rβ binding proteins of embodiments 1-13, wherein X¹⁰⁰ is present.

In a fifteenth embodiment, the present invention includes any one of the IL-2Rβ binding proteins of embodiment 14, wherein X¹⁰⁰ is serine.

In an sixteenth embodiment, the present invention includes any one of the IL-2Rβ binding proteins of embodiments 1-13 wherein wherein X¹⁰⁰ is absent.

In a seventeenth embodiment, the present invention includes any one of the IL-2Rβ binding proteins of embodiments 1-10 wherein the amino acid sequence of SEQ ID NO:28 is selected from the group consisting of:

(SEQ ID NO: 30)
PKKKIQLHAEHARYDAEMILNIJ¹KLEDYAFNFELILEEIARLFESGJ²
Z¹J³EDEQEEMANAIITILTSWIRS;
(SEQ ID NO: 31)
PKKKIQLHAEHALYDAEMILKIJ¹KLEDYAFNFELILEEIARLFESGJ²
Z¹J³EDEQEEMANRIRKILEEWIRS;
(SEQ ID NO: 14)
PKKKIQLHAEHARYDAEMILNIVKTNSPPAEEKLEDYAFNFELILEEIA
RLFESGDQKDEAEKAKRMKEWMKRIKTTASEDEQEEMANAIITILTSWI
RS;
(SEQ ID NO: 15)
PKKKIQLHAEHALYDAEMILKIVKTNSPPAEEKLEDYAFNFELILEEIA
RLFESGDQKDEAEKAKRMKEWMKRIKTTASEDEQEEMANRIRKILEEWI
RS;
(SEQ ID NO: 34)
PKKKIQLYAEHARYDAEMILNIJ¹ELEDYAFNFELILEEIARLFESGJ²
Z¹J³EDEQEEMANAIITILTSWIRS;
(SEQ ID NO: 35)
PKKKIQLYAEHALYDAEMILKIJ¹ELEDYAFNFELILEEIARLFESGJ²
Z¹J³EDEQEEMANRIRKILEEWIRS;
(SEQ ID NO: 16)
PKKKIQLYAEHARYDAEMILNIVKTNSPPAEEELEDYAFNFELILEEIA
RLFESGDQKDEAEKAKRMKEWMKRIKTTASEDEQEEMANAIITILTSWI
RS;
and
(SEQ ID NO: 9)
PKKKIQLYAEHALYDAEMILKIVKTNSPPAEEELEDYAFNFELILEEIA
RLFESGDQKDEAEKAKRMKEWMKRIKTTASEDEQEEMANRIRKILEEWI
RS .

In an eighteenth embodiment, the present invention includes any one of the IL-2Rβ binding proteins of embodiments 1-17, wherein Z¹ is from 8 to about 100 amino acids in length.

In a nineteenth embodiment, the present invention includes any one of the IL-2Rβ binding proteins of embodiment 18, wherein Z¹ is from 8 to about 50 amino acids in length.

In a twentieth embodiment, the present invention includes any one of the IL-2Rβ binding proteins of embodiments 1-19, wherein Z¹ is at least 19 amino acids in length.

In a twenty-first embodiment, the present invention includes any one of the IL-2Rβ binding proteins of embodiment 20, wherein Z¹ comprises the amino acid sequence KDEAEKAKRMKEWMKRIKT (SEQ ID NO:6).

In a twenty-second embodiment, the present invention includes any one of the IL-2Rβ binding proteins of embodiments 1-21, wherein the amino acid linker are present.

In a twenty-third embodiment, the present invention includes any one of the IL-2Rβ binding proteins of embodiment 22, wherein J¹ is about 10 amino acids in length, J² is about 2 amino acids in length and J³ is about 3 amino acids in length. An exemplary sequence for J¹ is VKTNSPPAEE (SEQ ID NO:23). An exemplary sequence for J² is DQ and an exemplary sequence for J³ is TAS (SEQ ID NO:24).

In a twenty-fourth embodiment, the present invention includes any one of the IL-2Rβ binding proteins of embodiments 1-23, wherein the IL-2Rβ binding protein binds human IL-2Rβ with substantially the same or higher binding affinity (substantially the same or lower Kd) as compared to the binding affinity of IL-2 to human IL-2Rf3.

In a twenty-fifth embodiment, the present invention includes any one of the IL-2Rβ binding proteins of embodiment 24, wherein the IL-2Rβ binding protein binds human IL-2Rβ with higher binding affinity (lower Kd) as compared to the binding affinity of IL-2 to human IL-2Rβ (e.g., 30 fold, 50 fold, 100 fold, or 150 fold higher affinity).

In a twenty-sixth embodiment, the present invention includes any one of the IL-2Rβ binding proteins of embodiments 1-25, wherein, in the resultant protein, X^{8A} is tyrosine and X^{33A} is glutamic acid.

In a twenty-seventh embodiment, the present invention includes a non-naturally occurring interleukin-2 receptor β (IL-2Rβ) binding protein that has reduced binding affinity to the human IL-2 gamma receptor as compared to IL-2 and comprises domains D1, D2, D3, and D4, wherein: D1 comprises an amino acid sequence at least 70% identical to the amino acid sequence: X¹X²X³KIQLYAEHARYDAEMILNI (SEQ ID NO:38), wherein

D1 comprises a glutamic acid at position 17 and X¹, X², and X³ can be any amino acid and can be present or absent; D2 is a helical-peptide of at least 8 amino acids in length; D3 comprises an amino acid sequence at least 70% identical to the amino acid sequence: ELEDYAFNFELILEEIARLFESG (SEQ ID NO:2); and D4 comprises an amino acid sequence at least 70% identical to the amino acid sequence: EDEQEEMANAIITILX⁹⁵ASWIRX¹⁰⁰ (SEQ ID NO:40) provided that D4 comprises an arginine at position 20 and, in D4, X^{95A} is serine, threonine, or glutamic acid; and X¹⁰⁰ can be any amino acid and can be present or absent; wherein D1, D2, D3, and D4 may be in any order in the polypeptide; and wherein amino acid linkers may be present between any of the domains. Position numbering for D1 is according to SEQ ID NO:38 and for the purpose of position numbering, X¹ at the N terminus of SEQ ID NO:38 is designated as position 1. Position numbering for D4 is according to SEQ ID NO:40 and the glutamic acid at the N terminus of SEQ ID NO:40 is designated as position 1.

In a twenty-eighth embodiment, the present invention includes a non-naturally occurring interleukin-2 receptor β (IL-2Rβ) binding protein that has reduced binding affinity to the human IL-2 gamma receptor as compared to IL-2 and comprises domains D1, D2, D3, and D4, wherein: D1 comprises an amino acid sequence at least 70% identical to SEQ ID NO:38, wherein D1 comprises a glutamic acid at position 17, and X¹, X², and X³ can be any amino acid and can be present or absent; D2 is a helical-peptide of at least 8 amino acids in length; D3 comprises an amino acid sequence at least 70% identical to the amino acid sequence of SEQ ID NO:2; and D4 comprises an amino acid sequence at least 70% identical to the amino acid sequence: EDEQEEMANRIRKILX⁹⁵AEWIRX¹⁰⁰ (SEQ ID NO: 41) provided that D4 comprises an arginine at position 20, and in D4, X^{95A} is serine, threonine, or glutamic acid ; and X¹⁰⁰ can be any amino acid and can be present or absent; wherein D1, D2, D3, and D4 may be in any order in the polypeptide; and wherein amino acid linkers may be present between any of the domains. Position numbering for D1 is according to SEQ ID NO:38 and for the purpose of position numbering, X¹ at the N terminus of SEQ ID NO:38 is designated as position 1. Position numbering for D4 is according to SEQ ID NO:41 and the glutamic acid at the N terminus of SEQ ID NO:41 is designated as position 1.

In a twenty-ninth embodiment, the present invention includes a non-naturally occurring interleukin-2 receptor β (IL-2Rβ) binding protein that has reduced binding affinity to the human IL-2 gamma receptor as compared to IL-2 and comprises domains D1, D2, D3, and D4, wherein: D1 comprises an amino acid sequence at least 70% identical to the amino acid sequence: X¹X²X³KIQLYAEHALYDAEMILKI (SEQ ID NO:42), wherein D1 comprises a glutamic acid at position 17, and X¹, X², and X³ can be any amino acid and can be present or absent; D2 is a helical-peptide of at least 8 amino acids in length; D3 comprises an amino acid sequence at least 70% identical to the amino acid sequence of SEQ ID NO:2; and D4 comprises an amino acid sequence at least 70% identical to SEQ ID NO:40 provided that D4 comprises an arginine at position 20 and, in D4, X^{95A} is serine, threonine, or glutamic acid; and X¹⁰⁰ can be any amino acid and can be present or absent; wherein D1, D2, D3, and D4 may be in any order in the polypeptide; and wherein amino acid linkers may be present between any of the domains. Position numbering for D1 is according to SEQ ID NO:42 and for the purpose of position numbering, X¹ at the N terminus of SEQ ID NO:42 is designated as position 1. Position numbering for D4 is according to SEQ ID NO:40 and the glutamic acid at the N terminus of SEQ ID NO:40 is designated as position 1.

In a thirtieth embodiment, the present invention includes a non-naturally occurring interleukin-2 receptor β (IL-2Rβ) binding protein that has reduced binding affinity to the human IL-2 gamma receptor as compared to IL-2 and comprises domains D1, D2, D3, and D4, wherein: D1 comprises an amino acid sequence at least 70% identical to SEQ ID NO:42 wherein D1 comprises a glutamic acid at position 17, and X¹, X², and X³ can be any amino acid and can be present or absent; D2 is a helical-peptide of at least 8 amino acids in length; D3 comprises an amino acid sequence at least 70% identical the amino acid sequence of SEQ ID NO:2; and D4 comprises an amino acid sequence at least 70% identical to SEQ ID NO:41 provided that D4 comprises an arginine at position 20 and, in D4, X^{95A} is serine, threonine, or glutamic acid; and X¹⁰⁰ can be any amino acid and can be present or absent; wherein D1, D2, D3, and D4 may be in any order in the polypeptide; and wherein amino acid linkers may be present between any of the domains. Position numbering for D1 is according to SEQ ID NO:42 and for the purpose of position numbering, X¹ at the N terminus of SEQ ID NO:42 is designated as position 1. Position numbering for D4 is according to SEQ ID NO:41 and the glutamic acid at the N terminus of SEQ ID NO:41 is designated as position 1.

In a thirty-first embodiment, the present invention includes any one of the IL-2Rβ binding proteins of embodiment 27, wherein D1 comprises an arginine at position 13 and X^{95A} of D4 is threonine.

In a thirty-second embodiment, the present invention includes any one of the IL-2Rβ binding proteins of embodiment 30, wherein D4 comprises an arginine at position 10, an arginine at position 12, a lysine at position 13, a glutamic acid at position 17 and X^{95A} of D4 is glutamic acid.

In a thirty-third embodiment, the present invention includes any one of the IL-2Rβ binding proteins of embodiments 27-32 wherein D1 comprises a tyrosine at position 8 and D3 comprises a glutamic acid at position 1, wherein the position numbering for D3 is according to SEQ ID NO:2.

In a thirty-fourth embodiment, the present invention includes any one of the IL-2Rβ binding proteins of embodiments 27-33 wherein D1 comprises a lysine at position 21.

In a thirty-fifth embodiment, the present invention includes any one of the IL-2Rβ binding proteins of embodiments 27-34 wherein D1 comprises a histidine at position 11 and a tyrosine at position 14 and D3 comprises a tyrosine at position 5, a phenylalanine at position 7, an asparagine at position 8, a leucine at position 11, and an isoleucine at position 12, wherein the position numbering for D3 is according to SEQ ID NO:2.

In a thirty-sixth embodiment, the present invention includes any one of the IL-2Rβ binding proteins of embodiments 27-34 wherein D1 comprises a histidine at position 11 and a tyrosine at position 14 and D3 comprises a tyrosine at position 5, a phenylalanine at position 7, an asparagine at position 8, a leucine at position 11, and an isoleucine at position 12, wherein the position numbering for D3 is according to SEQ ID NO:2.

In a thirty-seventh embodiment, the present invention includes any one of the IL-2Rβ binding proteins of embodiment 36, wherein D1 comprises a leucine at residue 7, a methionine at residue 18 and D3 comprises aspartic acid at position 4, glutamic acid at position 14, and glutamic acid at position 15, wherein the position numbering for D3 is according to SEQ ID NO:2.

In a thirty-eighth embodiment, the present invention includes any one of the IL-2Rβ binding proteins of embodiments 27-37, wherein D2 is from 8 to about 100 amino acids in length.

In a thirty-ninth embodiment, the present invention includes any one of the IL-2Rβ binding proteins of embodiments 27-38, wherein D2 is at least 19 amino acids in length.

In a fortieth embodiment, the present invention includes any one of the IL-2Rβ binding proteins of embodiment 39, wherein D2 comprises an amino acid sequence at least 80%, at least, 85%, at least 90%, at least 95%, or 100% identical to SEQ ID NO:6.

In a forty-first embodiment, the present invention includes any one of the IL-2Rβ binding proteins of embodiment 40, wherein D2 comprises the amino acid sequence set forth in SEQ ID NO:6.

In a forty-second embodiment, the present invention includes any one of the IL-2Rβ binding proteins of embodiments 27-41, wherein X¹, X² and X³ are present.

In a forty-third embodiment, the present invention includes any one of the IL-2Rβ binding proteins of embodiment 42, wherein Xi, X² and X³ are PKK, respectively.

In a forty-fourth embodiment, the present invention includes any one of the IL-2Rβ binding proteins of embodiments 27-41, wherein X¹, X² and X³ are absent.

In a forty-fifth embodiment, the present invention includes any one of the IL-2Rβ binding proteins of embodiments 27-44, wherein the order of the four domains in the final binding protein is D1-D3-D2-D4.

In a forty-sixth embodiment, the present invention includes any one of the IL-2Rβ binding proteins of embodiments 27-41, wherein X¹⁰⁰ is present.

In a forty-seventh embodiment, the present invention includes any one of the IL-2Rβ binding proteins of embodiment 46, wherein X¹⁰⁰ is serine.

In a forty-eighth embodiment, the present invention includes any one of the IL-2Rβ binding proteins of embodiments 27-41, wherein X¹⁰⁰ is absent.

In a forty-ninth embodiment, the present invention includes any one of the IL-2Rβ binding proteins of embodiments 27-41, wherein D1 comprises an amino acid sequence having at least 80% identity to the amino acid sequence set forth in SEQ ID NO: 38 or SEQ ID NO:42, D3 comprises an amino acid sequence at least 80% identical to the amino acid sequence set forth in SEQ ID NO: 2, and D1 comprises an amino acid sequence having at least 80% identity to the amino acid sequence set forth in SEQ ID NO: 40 or SEQ ID NO:41.

In a fiftieth embodiment, the present invention includes any one of the IL-2Rβ binding proteins of embodiment 49, wherein D1 comprises an amino acid sequence having at least 90% identity to the amino acid sequence set forth in SEQ ID NO: 38 or SEQ ID NO:42, D3 comprises an amino acid sequence at least 90% identical to the amino acid sequence set forth in SEQ ID NO: 2, and D1 comprises an amino acid sequence having at least 90% identity to the amino acid sequence set forth in SEQ ID NO: 40 or SEQ ID NO:41.

In a fifty-first embodiment, the present invention includes any one of the IL-2Rβ binding proteins of embodiment 49, wherein D1 comprises an amino acid sequence having at least 95% identity to the amino acid sequence set forth in SEQ ID NO: 38 or SEQ ID NO:42, D3 comprises an amino acid sequence at least 95% identical to the amino acid sequence set forth in SEQ ID NO: 2, and D1 comprises an amino acid sequence having at least 95% identity to the amino acid sequence set forth in SEQ ID NO: 40 or SEQ ID NO:41.

In a fifty-second embodiment, the present invention includes any one of the IL-2Rβ binding proteins of embodiments 27-51, wherein D1 comprises an amino acid sequence 100% identical to the amino acid sequence set forth in SEQ ID NO: 38 or SEQ ID NO:42, D3 comprises an amino acid sequence 100% identical to the amino acid sequence set forth in SEQ ID NO: 2, and D1 comprises an amino acid sequence 100% identical to the amino acid sequence set forth in SEQ ID NO: 40 or SEQ ID NO:41.

In a fifty-third embodiment, the present invention includes any one of the IL-2Rβ binding proteins of embodiments 49-51, wherein the percent of identity of D1 is to SEQ ID NO:38 and the percent identity of D4 is to SEQ ID NO:40.

In a fifty-fourth embodiment, the present invention includes any one of the IL-2Rβ binding proteins of embodiments 49-51, wherein the percent of identity of D1 is to SEQ ID NO:42 and the percent identity of D4 is to SEQ ID NO:41.

In a fifty-fifth embodiment, the present invention includes any one of the IL-2Rβ binding proteins of embodiments 49-51, wherein the percent of identity of D1 is to SEQ ID NO:42 and the percent identity of D4 is to SEQ ID NO:40.

In a fifty-sixth embodiment, the present invention includes any one of the IL-2Rβ binding proteins of embodiments 27-54, wherein amino acid linkers are present between the domains.

The present invention includes any one of the IL-2Rβ binding proteins of embodiments 27-56, wherein the IL-2Rβ binding protein binds human IL-2Rβ with substantially the same or higher binding affinity (substantially the same or lower Kd) as compared to the binding affinity of IL-2 to human IL-2R13.

The present invention includes any one of the IL-2Rβ binding proteins of embodiments 27-56, wherein the IL-2Rβ binding protein binds human IL-2Rβ with higher binding affinity (lower Kd) as compared to the binding affinity of IL-2 to human IL-2R13 (e.g., 30 fold, 50 fold, 100 fold, or 150 fold higher affinity).

As used herein, the term “Neo-2/15 variant protein” refer to the de novo protein mimic Neo-2/15 wherein specific substitutions to the Neo-2/15 protein have been made. The Neo-2/15 variant proteins are characterized by amino acid insertions, deletions, substitutions and modifications at one or more sites in or at the other residues of the Neo-2/15 polypeptide chain. In accordance with this disclosure, any such insertions, deletions, substitutions and modifications result in Neo-2/15 variants that retain IL-2Rβ binding activity. Exemplary variant proteins can include substitutions of 1, 2, 3, 4, 5, 6, 7, 8, 9, 10, 15, 20 or more amino acids. Substitutions can be natural or non-natural amino acids. Exemplary Neo-2/15 variant proteins are at least about 50%, at least about 65%, at least about 70%, at least about 80%, at least about 85%, at least about 87%, at least about 90%, at least about 91%, at least about 92%, at least about 93%, at least about 94%, at least about 95%, at least about 96%, or at least about 97% identical to Neo-2/15. The mutations therein can consist of a change in the number or content of amino acid residues. For example, the variant Neo-2/15 can have a greater or a lesser number of amino acid residues than Neo-2/15. Alternatively, or in addition, an exemplary variant protein can contain a substitution of one or more amino acid residues that are present in Neo-2/15.

In a fifty-seventh embodiment, the present invention includes a Neo-2/15 variant protein that has reduced binding affinity to the IL-2R gamma receptor as compared to Neo-2/15, wherein said Neo-2/15 variant comprises amino acid substitutions at positions 17, 95 and 99 wherein said substitutions are selected in order to decrease binding affinity the IL-2R gamma receptor as compared to Neo-2/15, wherein said numbering is in reference to Neo-2/15.

In a fifty-eighth embodiment, the present invention includes a Neo-2/15 variant protein that has reduced binding affinity to the IL-2R gamma receptor as compared to Neo-2/15, wherein said Neo-2/15 variant comprises amino acid substitutions L17E, Q95T or Q95E, or Q95S and F99R, wherein numbering is in reference to Neo-2/15.

In a fifty-ninth embodiment, the present invention includes the Neo-2/15 variant protein of any one of embodiments 57-58 having an amino acid substitution at position 13, wherein numbering is in reference to Neo-2/15.

In a sixtieth embodiment, the present invention includes the Neo-2/15 variant protein of embodiment 59 comprising the amino acid substitution L13R.

In a sixty-first embodiment, the present invention includes the Neo-2/15 variant protein of any one of embodiments 57-60 comprising one or more amino acid substitutions at positions 21, 89, 91, 92, or 96, wherein numbering is in reference to Neo-2/15.

In a sixty-second embodiment, the present invention includes the Neo-2/15 variant protein of embodiment 61 comprising one or more amino acid substitutions N21K, A89R, I91R, T92K, or S96E.

In a sixty-third embodiment, the present invention includes the Neo-2/15 variant protein of any one of embodiments 57-62 comprising amino acid substitutions at positions 89, 91, 92, and 96, wherein numbering is in reference to Neo-2/15.

In a sixty-fourth embodiment, the present invention includes the Neo-2/15 variant protein of embodiment 63 comprising the amino acid substitutions A89R, I91R, T92K, and S96E.

In a sixty-fifth embodiment, the present invention includes the Neo-2/15 variant protein of any one of embodiments 57-64 comprising an amino acid substitution at position 21, wherein numbering is in reference to Neo-2/15.

In a sixty-sixth embodiment, the present invention includes the Neo-2/15 variant protein of embodiment 65 comprising the amino acid substitution N21K.

In a sixty-seventh embodiment, the present invention includes the Neo-2/15 variant protein of any one of embodiments 57-66 comprising the amino acid substitution Q95E.

In a sixty-eighth embodiment, the present invention includes the Neo-2/15 variant protein of any one of embodiments 57-66 comprising the amino acid substitution Q95T.

In a sixty-ninth embodiment, the present invention includes the Neo-2/15 variant protein of any one of embodiments 57-68 further comprising one or more mutations that increase binding affinity to human IL-2Rβ as compared to Neo-2/15.

In a seventieth embodiment, the present invention includes the Neo-2/15 variant protein of any one of embodiments 57-68 further comprising a H8Y and K33E mutation, wherein numbering is in reference to Neo-2/15.

The present invention includes any one of the Neo-2/15 variant proteins of embodiments 57-70, wherein the Neo-2/15 variant protein binds human IL-2Rβ with substantially the same or higher binding affinity (substantially the same or lower Kd) as compared to the binding affinity of IL-2 to human IL-2R13.

The present invention includes any one of the Neo-2/15 variant proteins of embodiments 57-70 wherein the Neo-2/15 variant protein binds human IL-2Rβ with higher binding affinity (lower Kd) as compared to the binding affinity of IL-2 to human IL-2R13 (e.g., 30 fold, 50 fold, 100 fold, or 150 fold higher affinity).

In a seventy-first embodiment, the present invention includes the proteins of any one of embodiments 1-70 wherein the IL-2 receptor is a human IL-2 receptor.

### Polypeptide Variability

In order to retain the optimal binding and functional characteristics of the polypeptides, in some embodiments, it may be desirable that no amino acids are added or deleted within domains D1, D3, and D4 of the polypeptides of the present invention. The teachings provided herein can be used to determine the optimal sites for mutating amino acid residues and retaining their desired functional characteristics, i.e., diminished binding to IL-2RG and retained binding to IL-2Rβ.

The term “identity”, as used herein in reference to polypeptide sequences, refers to the subunit sequence identity between two molecules. When a subunit position in both molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained (including gaps if necessary). Identity may be calculated, in various embodiments, using published techniques and widely available computer programs, such as the GCG program package (Devereux et al., Nucleic Acids Res. 12:387, 1984), BLASTP, BLASTN, FASTA (Atschul et al., J. Molecular Biol. 215:403, 1990). Sequence identity can be measured, for example, using sequence analysis software such as the Sequence Analysis Software Package of the Genetics Computer Group at the University of Wisconsin Biotechnology Center (1710 University Avenue, Madison, Wis. 53705), using the default parameters. Unless indicated otherwise, percent identity is determined across the length of the reference sequence.

In some aspects, amino acid substitutions relative to the reference peptide domains may be conservative amino acid substitutions. As used herein, “conservative amino acid substitution” means a given amino acid can be replaced by an amino acid having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity is retained. Amino acids can be grouped according to similarities in the properties of their side chains (see, e.g., A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala to Gly or to Ser; Arg to Lys; Asn to Gln or to H is; Asp to Glu; Cys to Ser; Gln to Asn; Glu to Asp; Gly to Ala or to Pro; His to Asn or to Gln; Ile to Leu or to Val; Leu to Ile or to Val; Lys to Arg, to Gln or to Glu; Met to Leu, to Tyr or to Ile; Phe to Met, to Leu or to Tyr; Ser to Thr; Thr to Ser; Trp to Tyr; Tyr to Trp; and/or Phe to Val, to Ile or to Leu.

In some aspects the following teachings can be used to select amino acid substitutions for D1, D2, D3 and D4.

One representative sequence for D1 is set forth in SEQ ID NO:1 as KIQLYAEHAL YDAX₁₇MILX₂₁I . In some aspects, the amino acid at position 1 is K or if substituted is D, E, N, P, R, or W; the amino acid at position 2 is I or, if substituted, is D, E, H, K, L, M, or S; the amino acid at position 3 is Q or, if substituted, is A, D, E, G, L, P, S, or W; the amino acid at position 4 is L or, if substituted, is D, E, Q, or Y; the amino acid at position 5 is Y or, if substituted, is H or F; the amino acid at position 6 is A or, if substituted, is C, F, or P; the amino acid at position 7 is E or, if substituted, is C, D, F, K, or P; the amino acid at position 8 is H or, if substituted, is D or F; the amino acid at position 9 is A or, if substituted is D, E, P, S, T or V; the amino acid at position 11 is Y or, if substituted, is F, R or W; the amino acid at position 12 is D or, if substituted, is E, N or Y; the amino acid at position 13 is A or, if substituted, is C, L, M or S; the amino acid at position 15 is M, or if substituted is G, Q, or Y; the amino acid at position 16 is I or, if substituted is L, M, P, Q or V; the amino acid at position 17 is L or, if substituted is A, K, M, Q, R, or S; the amino acid at position 19 is I or, if substituted is D, E, K, M, N,W, or Y. In some aspects, exemplary amino acid residues for position 10 are L, H, I, M, P, R, V, or W. In some aspects, X₂₁ is N, G, K, P, R, S, or W. With respect to position X₁₇, it is selected so as to reduce binding to the gamma interface. In some aspects, X₁₇ is E or another amino acid residue that acts to interfere with binding to the gamma interface. D1 optionally will comprise amino acids N-terminal to the lysine at position 1. In some aspects, the polypetide will comprise at least 3 amino acid N terminal to the lysine and the amino acids will be proline-lysine-lysine-. In some aspects, instead of proline, the amino acid will be selected from A, F, I, L, M, Q, R, S or W. In some aspects, instead of lysine adjacent to the proline, the amino acid will be selected from A, D, E, G, or or V. In some aspects, instead of lysine adjacent to position 1, the amino acid will be selected from D, E, F, or W.

One representative sequence for D3 is set forth in SEQ ID NO:2 as ELEDYAFN FELILEEIAR LFESG. In some aspects, the amino acid at position 1 is E or, if substituted, is C, Y, or D; the amino acid at position 2 is L or, if substituted, is A; the amino acid at position 3 is E or, if substituted, is D, G, K, M or T; the amino acid at position 4 is D or, if substituted, is E, N or Y; the amino acid at position 5 is Y or, if substituted is C, D, G, or T; the amino acid at position 6 is A or, if substituted, is F, H, S, V, W, or Y; the amino acid at position 7 is F or, if substituted, is A, I, M, T, V, or Y; the amino acid at position 8 is N, or if substituted, is D, K, S, or T; the amino acid at position 9 is F or, if substituted, is A, C, G, L, M, S, or V; the amino acid at position 10 is E or, if substituted, is C, H, K, L, R, S, T, or V; the amino acid at position 11 is L or, if substituted, is F, I, M or Y; the amino acid at position 12 is I or, if substituted, is L, T, or Y; the amino acid at position 13 is L of, if substituted, is F, K, M, S, or V; the amino acid at position 14 is E or, if substituted, is A, D, F, G, I, N, P, Q, S or T; the amino acid at position 15 is E or, if substituted, is A, F, H, S, or V; the amino acid at position 16 is I or, if substituted, is C, L, M, V, or W; the amino acid at position 17 is A or, if substituted, is D, G, S, T, or V; the amino acid at position 18 is R or, if substituted, is H, K, L, or N; the amino acid at position 19 is L or, if substituted, is C, D, G, L, Q, R, T or W; the amino acid at position 20 is F or, if substituted, is D, M, N, or W; the amino acid at position 21 is E or, if substituted, is A, C, F, G, M, S or Y; the amino acid at position 22 is S or, if substituted, is D, E, G, H, L, M, R, T, V, or W, and the amino acid at position 23 is G of, if substituted, is A, D, K, N, S, or Y.

One representative sequence for D4 is set forth in SEQ ID NO:3: EDEQEEMANX₈₉I X₉₁X₉₂ILX₉₅X₉₆WIX₉₉S. In some aspects, the amino acid at position 1 is E or, if substituted, is D, G, K, or V; the amino acid at position 2 is D or, if substituted, is I, M, or S; the amino acid at position 3 is E or, if substituted, is G, H, or K; the amino acid at position 4 is Q or, if substituted, is E, G, I, K, R, or S; the amino acid at position 5 is E or, if substituted, is A, D, G, H, S, or V; the amino acid at position 6 is E, or if substituted, is C, D, G, I, M, Q, R, T or V; the amino acid at position 7 is M or, if substituted, is C, E, L, P, R, or T; the amino acid at position 8 is A or, if substituted, is F, L, M, or W; the amino acid at position 9 is N or, if substituted, is A, G, L, Q, R, or T; the amino acid at position 11 is I or, if substituted, is M, N, S, V, or W; the amino acid at positionl4 is I or, if substituted, is L, P, T or Y; the amino acid at position 15 is L or, if substituted, is F, G, I, M, N, or V; the amino acid at position 18 is W or, if substituted, is K, Q, or T; the amino acid at position 19 is I or, if substituted, C, G, or N; the amino acid at position 21 is S or, if substituted, is A, F, G, H, or Y. X₈₉, X₉₁, X₉₂, X₉₅, and X₉₆ can be as described herein.

One representative sequence for D2 is set forth in SEQ ID NO:6 KDEAEK AKRMKEWMKR IKT . In some aspects, the amino acid at position 1 is A, H, L, M, R, S, V, K, or C; the amino acid at position 2 is A, D, E, Q, R, S, T, V, W, Y or C; the amino acid at position 3 is C, E, G, K, L, N, Q, R, or W; the amino acid at position 4 is A, F, G, N, S, T, V, or Y; the amino acid at position 5 is A, E, G, I, M, R, V, or C; the amino acid at position 6 is C, E, K, L, N, R, or V; the amino acid at position 7 is A, C, E, I, L, S, T, V, or W; the amino acid at position 8 is H, K, L, M, S, T, W, or Y; the amino acid at position 9 is A, I, L, M, Q, S, R or C; the amino acid at position 10 is A, I, M, S, W, or Y; the amino acid at position 11 is C, I, K, L, S, or V; the amino acid at position 12 is C, E, K, L, P, Q, R, or T; the amino acid at position 13 is A, D, H, N or W; the amino acid at position 14 is A, C, G, I, L, S, T, V, or M; the amino acid at position 15 is A, E, G, I, K, L, M, R, or V; the amino acid at position 16 is G, H, L, R, S, T, V or C; the amino acid at position 17 is A, I, L, or V; the amino acid at position 18 is A, C, D, E, G, H, I, K, M or S; and the amino acid at position 19 is D, E, G, L, N, V or T.

In some aspects, a polypeptide of the present invention is multivalent (e.g. bivalent, trivalent, tetravalent). which means it comprises two or more IL-2Rβ binding units and as such, can bind two or more IL-2 receptors. In some such aspects, two or more amino acid sequences described herein are joined together via a linker to create a multivalent polypeptide.

As noted above, exemplary polypeptides of the present invention comprise 4 domains, D1, D2, D3, and D4. In some aspects, the 4 domains joined together are 85-300 amino acids in length, 85-200 amino acids in length, 85-120 amino acids in length, 90-300 amino acids in length, 90-200 amino acids in length, 90-120 amino acids in length, 95-300 amino acids in length, 95-200 amino acids in length, or 95-120 amino acids in length.

In a further aspect, the present disclosure provides antibodies that selectively bind to the polypeptides of the disclosure. The antibodies can be polyclonal, monoclonal antibodies, humanized antibodies, and fragments thereof, and can be made using techniques known to those of skill in the art. As used herein, “selectively bind” means preferential binding of the antibody to the protein of the disclosure, as opposed to one or more other biological molecules, structures, cells, tissues, etc., as is well understood by those of skill in the art.

### Activity of the Exemplary Polypeptides of the Present Invention

Polypeptides of the present invention are designed to have differential activity as compared to IL-2 and/or Neo-2/15. Certain polypeptides of the present invention are IL-2 and/or IL-15 receptor antagonists (e.g., hIL-2 and hIL-15 receptor antagonists). In exemplary embodiments, polypeptides of the present invention inhibit IL-2 binding to the IL-2 receptor Byc heterodimer in vitro and/or in vivo. In exemplary embodiments, polypeptides of the present invention inhibit IL-2 signaling in vitro and/or in vivo. In exemplary embodiments, the inhibition of IL-2 binding and/or signaling is selective. In some aspects, inhibition of IL-2 binding and/or signaling is more pronounced in CD25 negative cells or cells with low or medium levels of CD25 than in cells with high levels of CD25. In some aspects, IL-2 binding and/or signaling are inhibited in CD4⁺ T CD⁺ T cells that are CD25- but to a lesser extent in CD25⁺ T regulatory cells. In some aspects, IL-2 binding and/or signaling are not inhibited or minimally inhibited in CD25⁺ T regulatory cells.

In exemplary embodiments, polypeptides of the present invention have a reduced ability to stimulate STATS phosphorylation as compared to Neo-2/15 and/or IL-2 in select cell types. In some aspects, stimulation of STATS phosphorylation by a polypeptide of the present invention is at a level that is at least 50% less, at least 75% less, at least 90% less, or at least 95% less than the level of STATS phosphorylation stimulated by IL-2 in that same cell type. In some aspects, stimulation of STATS phosphorylation by a polypeptide of the present invention is at level that is at least 50% less, at least 75% less, at least 90% less, or at least 95% less than the level of STATS phosphorylation stimulated by Neo-2/15 in that same cell type. In some aspects, polypeptides of the present invention do not detectably stimulate STAT5 phosphorylation in select IL-2Rβγc positive cell types. In some embodiments, the cell is a T cell (for example, a CD⁺CD25⁺ T cell, a CD4⁺ CD25⁻ T cell, or a NK cell). STAT5 signaling can be measured by any method known in the art, including, for example, using a method shown in the examples. For example, STAT5 phosphorylation can be measured using antibodies specific for phosphorylated STAT5 and flow cytometry analysis. In some aspects, the polypeptides of the present invention inhibit or prevent IL-15 from stimulating STAT5 phosphorylation in select cell types (such as a CD8+ T cell or NK cell).

In some aspects, polypeptides of the present invention block more than 20%, more than 30%, more than 40%, more than 50%, more than 60%, more than 70% more than 80%, or more than 90% of IL-2 binding to IL-2R as determined using assays known in the art. In some embodiments, IL-2R is the intermediate affinity IL-2 receptor. In some aspects, polypeptides of the present invention block more than 20%, more than 30% more than 40%, more than 50%, block more than 50%, more than 60%, more than 70%, more than 80%, or more than 90% of IL-2 binding to IL-2R in CD4⁺ CD25⁻ cells, and CD8⁺CD25⁻ cells but not in T regulatory cells.

Included herein are polypeptides that have (i) decreased (including negligible) ability to bind hIL-2Rγ_{c} and hIL-2RBγ_{c} (ii) increased human IL-2 beta receptor binding affinity as compared to IL-2 and/or Neo-2/15, and/or (iii) act as IL-2R and/or IL-15R antagonists. In some aspects, such exemplary polypeptides act as competitive inhibitors of IL-2 and/or IL-15. Without intending to be bound by any particular theory, polypeptides of the invention may function by interfering with the binding of hIL-2 to the hIL-2 receptor and inhibiting the heterodimerization of the hIL-2R beta and common gamma receptors. Because IL-15 signals via this pathway as well, exemplary polypeptides of the present invention may, in some embodiments, act as dual antagonists for both IL-2 and IL-15.

Polypeptides of the present invention possess the improved characteristics of de novo proteins with respect to improved stability as compared to native proteins and variants thereof. Benefits of increased stability include the elimination of requirement for cold chain storage and a tolerance of mutations, genetic fusions, and chemical modifications.

### Nucleic Acids, Expression Vectors and Host Cells.

In a further aspect, the present invention provides nucleic acids, including isolated nucleic acids, encoding polypeptides of the present invention. The nucleic acid sequence may comprise RNA or DNA. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present invention provides recombinant expression vectors comprising the nucleic acid of any aspect of the invention. In some aspects the nucleic acid is operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector. In some embodiments, the expression vector can be administered in the methods of the disclosure to express the polypeptides in vivo for therapeutic benefit.

In a further aspect, the present disclosure provides host cells that comprise the nucleic acids and recombinant expression vectors disclosed herein, wherein the host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (R.I. Freshney. 1987. Liss, Inc. New York, NY)). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

### Fusion Proteins and Conjugates

Exemplary polypeptides of the present invention can be prepared as fusion or chimeric proteins that include polypeptide of the present invention and a heterologous polypeptide. In some embodiments, heterologous polypeptides can increase the circulating half-life of the resultant chimeric polypeptide in vivo, and may, therefore, further enhance the properties of the proteins of the present invention. In various embodiments, the polypeptide that increases the circulating half-life may be a serum albumin, such as human serum albumin, or the Fc region of an IgG subclass of antibodies. Exemplary Fc regions can include one or more mutations that inhibit complement fixation and/or Fc receptor binding or may be lytic, i.e., able to bind complement or to lyse cells via another mechanism, such as antibody-dependent complement lysis. In some embodiments, a Fc region is a naturally occurring or synthetic polypeptide that is homologous to the IgG C-terminal domain produced by digestion of IgG with papain. The fusion proteins can include the entire Fc region, or a smaller portion that retains a desired activity, such as the ability to extend the circulating half-life of a chimeric polypeptide of which it is a part. In addition, full-length or fragmented Fc regions can be variants of the wild-type molecule. That is, they can contain mutations that may or may not affect the function of the polypeptides. For example, a Fc region may have effector function or may be modified as to have one or more activities associated with effector function reduced or completely eliminated. Effector function refers to certain biological activities attributable to the Fc region of an immunoglobulin, which may vary with the immunoglobulin isotype. Examples of effector function include, but are not limited to, C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors, and B cell activation.

In some exemplary embodiments, the polypeptides of the present invention comprise an IgG1, IgG2, IgG3, or IgG4 Fc region. In some exemplary embodiments, the polypeptides of the present invention comprise a variant IgG1, IgG2, IgG3, or IgG4 Fc region. In some aspects, the variant Fc region lacks effector function.

In other embodiments, the polypeptides of the present invention may be linked to other types of stabilization compounds to promote an increased half-life in vivo, including but not limited to attachment of one or more polyethylene glycol chains (PEGylation). Accordingly, polypeptides of the present invention can comprise one or more stabilizing agents.

In that regard, polypeptides of the present invention can have amino acid substitutions that enable chemical conjugation with water soluble polymers (e.g., PEG) that increase circulating half-life compared to the protein alone. A “PEG” is a poly(ethylene glycol) molecule that is a water-soluble polymer of ethylene glycol. PEGs can be obtained in different sizes, and can also be obtained commercially in chemically activated forms that are derivatized with chemically reactive groups to enable covalent conjugation to proteins. Linear PEGs are produced in various molecular weights, such as PEG polymers of weight-average molecular weights of 5,000 daltons, 10,000 daltons, 20,000 daltons, 30,000 daltons, and 40,000 daltons. Branched PEG polymers have also been developed. Commonly-used activated PEG polymers are those derivatized with N-hydroxysuccinimide groups (for conjugation to primary amines such as lysine residues and protein N-termini), with aldehyde groups (for conjugation to N-termini), and with maleimide or iodoacetamide groups (for coupling to thiols such as cysteine residues). Methods of designing moieties for conjugation to PEG are known in the art. For example, addition of polyethylene glycol (“PEG”) containing moieties may comprise attachment of a PEG group linked to maleimide group (“PEG-MAL”) to a cysteine residue of the protein. Suitable examples of PEG-MAL are methoxy PEG-MAL 5 kD; methoxy PEG-MAL 20 kD; methoxy (PEG)2-MAL 40 kD; methoxy PEG(MAL)2 5 kD; methoxy PEG(MAL)2 20 kD; methoxy PEG(MAL)2 40 kD; or any combination thereof.

In some embodiments, a stabilization compound, including but not limited to a PEG-containing moiety, is linked at a cysteine residue in a polypeptide of the present invention. In some embodiments, the cysteine residue is present in the D2 domain. In some embodiments, the cysteine residue is present, for example, in any one of a number of positions in the D2 domain. In some such embodiments, the D2 domain is at least 19 amino acids in length and the cysteine residue is at positions 1, 2, 5, 9 or 16 relative to those 19 amino acids. In a further embodiment, the stabilization compound, including but not limited to a PEG-containing moiety, is linked to the cysteine residue via a maleimide group, including but not limited to linked to a cysteine residue present at amino acid residue 62 relative to Neo-2/15.

In some aspects, polypeptides of the present invention comprise a targeting domain. The targeting domain can direct cellular localization of the polypeptide of the present invention. For example, in some aspects, it might be desirable to target the polypeptides of the present invention to inflammatory cells involved in auto-immune disease.

In some embodiments, the targeting domain can bind to, for example, immune cell surface markers. In this embodiment, the target may be cell surface proteins on any suitable immune cell, including but not limited to CD8+ T cells, T-regulatory cells, dendritic cells, natural killer (NK) cells or macrophages. The targeting domain may target any suitable immune cell surface marker.

When a targeting domain is a polypeptide, the targeting domain can be any suitable polypeptide that bind to one or more targets of interest and can be attached or associated with a polypeptide of the present invention. In non-limiting embodiments, the targeting domain may include but is not limited to an scFv, a F(ab), a F(ab′)₂, a B cell receptor (BCR), a DARPin, an affibody, a monobody, a nanobody, diabody, an antibody (including a monospecific or bispecific antibody); a cell-targeting oligopeptide including but not limited to RGD integrin-binding peptides, de novo designed binders, aptamers, a bicycle peptide, conotoxins, small molecules such as folic acid, and a virus that binds to the cell surface. The targeting domain may be covalently or non-covalently bound to the protein.

In another embodiment, the targeting domain, when present, is a translational fusion with the protein. In this embodiment, the protein and the targeting domain may directly about each other in the translational fusion or may be linked by a polypeptide linker suitable for an intended purpose. Exemplary such linkers include, but are not limited, to those disclosed in WO2016178905, WO2018153865 (in particular, at page 13), and WO 2018170179 (in particular, at paragraphs [0316]-[0317]). Methods of making fusion proteins and conjugates are known in the art and not discussed herein in detail.

### Methods of Treatment

In certain embodiments, polypeptides described herein are useful for the treatment of one or more conditions wherein suppression of one or more IL-2 and/or IL-15 dependent functions is desirable.

The present disclosure provides, inter alia, methods for modulating an immune response in a subject by administering to the subject a polypeptide of the present invention.

As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), such as a response by a CD4 T cell, CD8 T cell, or B cell via their antigen-specific receptor. In some embodiments, an immune response is a T cell response, such as a CD4+ response or a CD8+response. Such responses by these cells can include, for example, cytotoxicity, proliferation, cytokine or chemokine production, trafficking, or phagocytosis, and can be dependent on the nature of the immune cell undergoing the response. In some embodiments of the compositions and methods described herein, an immune response being modulated is T-cell mediated. Methods of measuring an immune response are known in the art and include, for example, measuring pro-inflammatory cytokines such as IL-6, IL-12 and TNF-alpha as well as co-stimulatory molecules, such as CD80, CD86, and chemokine receptor.

The polypeptides of the present invention can be used, for example, to treat diseases associated with IL-15 and/or IL-2 activity. The polypeptides of the present invention can be used, for example, to treat a subject, e.g., a human subject, who is suffering from a disease associated with IL-15 and/or IL-2 activity. In some embodiments, the disease associated with IL-15 and/or IL-2 activity is an autoimmune disease. In some embodiments, the polypeptides of the present invention are used to treat an autoimmune disease in a subject. Autoimmune diseases include, but are not limited to the following: (1) a rheumatic disease such as rheumatoid arthritis, systemic lupus erythematosus, Sjogren's syndrome, scleroderma, mixed connective tissue disease, dermatomyositis, polymyositis, Reiter's syndrome or Behcet's disease (2) type II diabetes (3) an autoimmune disease of the thyroid, such as Hashimoto's thyroiditis or Graves' Disease (4) an autoimmune disease of the central nervous system, such as multiple sclerosis, myasthenia gravis, or encephalomyelitis (5) a variety of phemphigus, such as phemphigus vulgaris, phemphigus vegetans, phemphigus foliaceus, Senear-Usher syndrome, or Brazilian phemphigus, (6) psoriasis, (7) inflammatory bowel disease (e.g., ulcerative colitis or Crohn's Disease) and (8) celiac disease. In some embodiments, treatment of the subject is via administration of a polypeptide of the present invention.

In some embodiments, a polypeptides of the present invention can be used, for example, to treat a subject, e.g., a human subject, who has received a transplant of biological materials, such as an organ, tissue, or cell transplant. For example, the polypeptides of the invention may be particularly suitable in promoting graft survival (allograft or xenograft) and/or in treating patients with graft versus host disease. In some embodiments, treatment of the subject is via administration of a polypeptide of the present invention.

The polypeptides of the present invention can also be used, for example, as research tools to study the differential effects of I1-2 and/or IL-15 agonism and antagonism.

### Pharmaceutical Compositions

Pharmaceutical compositions can be formulated to improve the bioavailability of the polypeptides of the present invention upon administration of the composition to a patient. Such pharmaceutical compositions can take the form of solutions, suspensions, emulsion, microparticles, tablets, pills, pellets, capsules, capsules containing liquids, powders, sustained-release formulations, suppositories, emulsions, aerosols, sprays, suspensions, or any other form suitable for use. Examples of suitable pharmaceutical carriers are described in “Remington's Pharmaceutical Sciences” by E. W. Martin. It will be evident to those of ordinary skill in the art that the optimal dosage of the active ingredient(s) in the pharmaceutical composition will depend on a variety of factors. Relevant factors include, without limitation, the type of animal (e.g., human), the particular form of polypeptides of the present invention, the manner of administration, and the composition employed.

A pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous or particulate, so as to provide an aerosol composition useful in, e.g., inhalatory administration.

When intended for oral administration, the polypeptides of the present invention are preferably in solid or liquid form, where semi-solid, semi-liquid, suspension and gel forms are included within the forms considered herein as either solid or liquid. As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the composition is in the form of a capsule, e.g., a gelatin capsule, it can contain, in addition to materials of the above type, a liquid carrier such as polyethylene glycol, cyclodextrin or a fatty oil. The composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included. Also contemplated are delayed release capsule, including those with an enteric coating.

The liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. A parenteral composition can be enclosed in ampoule, a disposable syringe or a multiple-dose vial made of glass, plastic or other material. Physiological saline is an exemplary adjuvant. An injectable composition is preferably sterile.

In another aspect, the present disclosure provides pharmaceutical compositions, comprising one or more polypeptides of the present invention and a pharmaceutically acceptable carrier. The term “carrier” refers to a diluent, adjuvant or excipient, with which a polypeptides of the present invention is administered. The pharmaceutical composition may comprise, for example, in addition to the polypeptide of the disclosure (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate- 60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides of the present invention may be the sole active agent in the pharmaceutical composition, or the composition may further comprise one or more other active agents suitable for an intended use.

In order to treat disease, the polypeptides of the present invention are provided in a therapeutically effective amount. This refers to an amount of the polypeptide effective for treating the disease or having the desired effect. The data obtained from the cell culture assays and animal studies can be used in formulating a range of dosage for use in humans. Dosage regimens can be adjusted by clinicians to provide the optimum desired response (e.g., a therapeutic or prophylactic response). The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the polypeptides can include a single treatment or, can include a series of treatments.

An exemplary dosage range for the polypeptides of the present invention may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. In some embodiments, the recommended dose could be lower than 0.1 mcg/kg, especially if administered locally. In other embodiments, the recommended dose could be based on weight/m² (i.e. body surface area), and/or it could be administered at a fixed dose (e.g., .05-100 mg). The polypeptides can be delivered in a single bolus, or may be administered more than once (e.g., 2, 3, 4, 5, or more times) as determined by an attending physician.

The following examples are provided to describe certain embodiments of the invention provided herein and are not to be construed to as limiting.

### EXAMPLES

IL-2R beta binding proteins were recombinantly expressed and purified from E. coli. Genes encoding the designed protein sequences were synthesized and cloned into pET-28b(+) E. coli plasmid expression vectors (GenScript, N-terminal 6xHis tag and thrombin cleavage site). Plasmids were then transformed into chemically competent E. coli Lemo21 cells (NEB). Protein expression was performed using Terrific Broth and M salts, cultures were grown at 37° C. until OD6′ reached approximately 0.8, then expression was induced with 1 mM of isopropyl β-D-thiogalactopyranoside (IPTG), and temperature was lowered to 18° C. After expression for approximately 18 hours, cells were harvested and lysed with a Microfluidics M110P microfluidizer at 18,000 psi, then the soluble fraction was clarified by centrifugation at 24,000 g for 20 minutes. The soluble fraction was purified by Immobilized Metal Affinity Chromatography (Qiagen) followed by FPLC size-exclusion chromatography (Superdex 75 10/300 GL, GE Healthcare). The purified proteins were characterized by Mass Spectrum (MS) verification of the molecular weight of the species in solution (Thermo Scientific), Size Exclusion - MultiAngle Laser Light Scattering (SEC-MALLS) in order to verify monomeric state and molecular weight (Agilent, Wyatt), SDS-PAGE, and endotoxin levels.

Protein 1 (P1) is as set forth in SEQ ID NO: 14. It comprises the same sequence as Neo-2/15 except for the following mutations: L13R, L17E, Q95T, and F99R.

Protein 2 (P2) is at set forth in SEQ ID NO: 15. It comprises the same sequence as Neo-2/15 except for the following mutations: L17E, N21K, A89R, I91R, T92K, Q95E, S96E, and F99R.

Protein 3 (P3) comprises the same sequence as P1 except for two additional mutations: H8Y, and K33E and is as set forth in SEQ ID NO: 16.

Protein 4 (P4) comprises the same sequence as P2 except for two additional mutations: H8Y, and K33E and is as set forth in SEQ ID NO:9. P4 is also referred to herein as S4.

Protein 5 (P5) is as set forth in SEQ ID NO:39. It comprises the same sequence as Neo-2/15 except for the following mutations: L17E, N21K, A89K, I91R, T92K, Q95E, S96E, and F99R.

Protein 6 (P6) comprises the same sequence as P5 except for two additional mutations: Y8F and K33E and is as set forth in SEQ ID NO: 10.

Neo-2/15 used in the examples is as set forth in SEQ ID NO:2. As used herein PEGylated Neo-2/15 refers to a PEGylated variant of Neo-2/15 comprising an E62C substitution.

### Example 1

### Polypeptides of the Present Invention (P1 and P2) Demonstrated Little or No Binding to the Human IL-2 Gamma Receptor and Significantly Reduced pSTAT5 Signaling as Compared to PEGylated Neo-2/15

The affinity for hIL2 receptor gamma was calculated from binding and dissociation kinetics at different protein concentration. hIL2 receptor gamma was immobilized in the surface of anti-human IgG Fc biosensors (ForteBio). For this purpose, sensors were soaked in samples containing 20 nM of hIL2RG-fc chimera (AcroBiosystems) for 300 sec. Subsequently, sensors were dipped in five-fold serial dilutions (5000 nM-1.6 nM) of Neo-2/15, P1 or P2, and binding was measured for 600 sec. Finally, sensors were removed from the protein samples and dipped in buffer solutions to promote and measure dissociation (1500 sec). The buffer used to prepare all the samples and soak the sensors was HBS-EP+ from GE Healthcare, which contains 10 mM HEPES, 150 mM NaCl, 3 mM EDTA and 0.05% v/v surfactant P20, pH 7.4. Data were acquired at 30° C. using an Octet RED96e system (ForteBio) and processed using the instrument's integrated software. Kinetics were fitted to a 1:1 binding model after subtracting a buffer signal baseline and the Kd values were calculated. Kd values were estimated from response vs protein concentration plots and are shown in Table 1 below.

The affinity for hIL2 receptor beta was calculated from binding and dissociation kinetics at different protein concentration. hIL2 receptor beta was immobilized in the surface of anti-human IgG Fc biosensors (ForteBio). For this purpose, sensors were soaked in samples containing 20 nM of hIL2RB-fc chimera (AcroBiosystems) for 300 sec. Subsequently, sensors were dipped in five-fold serial dilutions (1000 nM — 0.32 nM) of Neo-2/15, P1 or P2, and binding was measured for 600 sec. Finally, sensors were removed from the protein samples and dipped in buffer solutions to promote and measure dissociation (1500 sec). The buffer used to prepare all the samples and soak the sensors was HBS-EP+from GE Healthcare, which contains 10 mM HEPES, 150 mM NaCl, 3 mM EDTA and 0.05% v/v surfactant P20, pH 7.4. Data were acquired at 30° C. using an Octet RED96e system (ForteBio) and processed using the instrument's integrated software. Kinetics were fitted to a 1:1 binding model after subtracting a buffer signal baseline and the Kd values were calculated. Kd values were estimated from response vs protein concentration plots and are shown in Table 1 below.

Approximately 2×10⁵ YT-1 CTLL-2 cells were plated in each well of a 96-well plate and re-suspended in RPMI complete medium containing serial dilutions of Neo-2/15, P1 or P2. Cells were stimulated for 15 min at 37° C. and immediately fixed by addition of formaldehyde to 1.5% and 10 min incubation at room temperature. Permeabilization of cells was achieved by resuspension in ice-cold 100% methanol for 30 min at 4° C. Fixed and permeabilized cells were washed twice with FACS buffer (phosphate-buffered saline [PBS] pH 7.2 containing 0.1% bovine serum albumin) and incubated with Alexa Fluor® 647-conjugated anti-STATS pY694 (BD Biosciences) diluted in FACS buffer for 2 hours at room temperature. Cells were then washed twice in FACS buffer and MFI was determined on a CytoFLEX flow cytometer (Beckman-Coulter). Dose-response curves were fitted to a logistic model and half-maximal effective concentration (EC5o values) were calculated using GraphPad Prism data analysis software after subtraction of the mean fluorescence intensity (MFI) of unstimulated cells and normalization to the maximum signal intensity. P1 and P2 demonstrated significantly reduced pSTAT5 signaling as compared to Neo-2/15.

TABLE 1

| | Binding to hIL2RB | Binding to hIL2RG | Induced Signaling |
|---|---|---|---|
| Protein | Kd (nM) | Kd (nM) | EC50 (nM) |
| Neo-2/15 | 2.9 | 277 | 0.12 |
| P1 | 17 | 1190 | 557.2 |
| P2 | 21 | No binding detected | 425.5 |

These results indicate that the interaction with hIL2RG was successfully disrupted in P1 and P2, nevertheless, the affinity for hIL2RB needs to be improved in order to obtain an ideal hIL2 antagonist.

### Example 2

### Polypeptides of the Present Invention (P3 and P4) Demonstrated Increased Binding to IL2 Receptor Beta and Decreased Binding to IL2 Receptor Gamma as Compared to Neo-2/15

The affinity for hIL2 receptor beta was calculated from binding and dissociation kinetics at different protein concentration. hIL2 receptor beta was immobilized in the surface of anti-human IgG Fc biosensors (ForteBio). For this purpose, sensors were soaked in samples containing 20 nM of hIL2RB-fc chimera (Symansis) for 300 sec. Subsequently, sensors were dipped in two-fold serial dilutions (200 nM — 6.1 uM) of Neo-2/15, P3 or P4, and binding was measured for 600 sec. Finally, sensors were removed from the protein samples and dipped in buffer solutions to promote and measure dissociation (1500 sec). The buffer used to prepare all the samples and soak the sensors was HBS-EP+from GE Healthcare, which contains 10 mM HEPES, 150 mM NaCl, 3 mM EDTA and 0.05% v/v surfactant P20, pH 7.4. Data were acquired at 30° C. using an Octet RED96e system (ForteBio) and processed using the instrument's integrated software. Kinetics were fitted to a 1:1 binding model after subtracting a buffer signal baseline and the Kd values were calculated. Kd values were estimated from response vs protein concentration plots and are shown in Table 2 below.

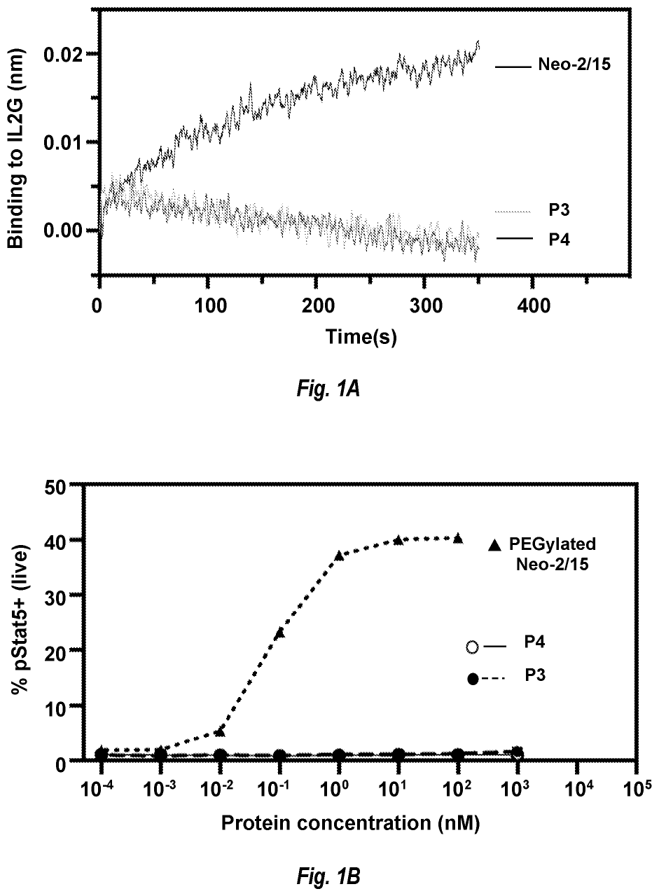

hIL2 receptor gamma binding kinetics were measured for Neo-2/15, P3 and P4. hIL2RG was immobilized in the surface of anti-human IgG Fc biosensors (ForteBio), for this purpose, sensors were soaked in samples containing 20 nM of hIL2RG-fc chimera (Symansis) for 300 sec. Subsequently, sensors were dipped in 200 nM of Neo215, P3 or P4 +200 nM hIL2RB-fc chimera (Symansis) , and binding was measured for 600 sec. All samples were prepared using HBS-EP+ buffer from GE Healthcare, which contains 10 mM HEPES, 150 mM NaCl, 3 mM EDTA and 0.05% v/v surfactant P20, pH 7.4. Data were acquired at 30° C. using an Octet RED96e system (ForteBio) and processed using the instrument's integrated software. See FIG. 1A and Table 2.

The mutations made to P1 and P2 to arrive at P3 and P4 resulted in proteins with higher affinity for hIL2RB.

TABLE 2

| | Binding to hIL2RB | Binding to hIL2G |
|---|---|---|
| Protein | Kd (nM) | Kd (nM) |
| Neo-2/15 | 2.9 | 277 |
| P3 | 0.2 | No binding detected |
| P4 | 0.06 | No binding detected |

### Example 3

### Polypeptides of the Present Invention (P3, P4) Demonstrated Little to No pSTAT5 Signaling on Human Pan T Cells

Human pan T cells were purified from PBMC and frozen for later use. Cells were thawed and rested overnight by culturing in X-VIVO 15 media (Lonza) without IL-2. The following day, the T cells were enumerated and plated at 50,000 cells per well in 96 well plates. P3 and P4 proteins were added to T cells starting at final concentration of 1 uM, titrating 8 points with 1:10 dilutions to 0.01 pM. As a positive signaling control, PEGylated Neo-2/15 was added to cells starting at a final concentration of 100 nM, titrating 8 points with 1:10 dilutions to 0.01 pM. Cultures were incubated at 37 degrees Celsius for 20 minutes before fixing cells in wells by adding formaldehyde to a final concentration of 1.5% by volume. Cells were fixed for 10 minutes at room temperature, centrifuged, and resuspended in 200 ul ice cold methanol to permeabilize the cells. Methanol was washed out twice with FACS buffer before adding anti-human pSTAT5 pY694 (BD Biosciences) diluted 1:25 in FACS buffer. Cells were incubated with antibody for 1 hour before washing twice and resuspending in 150 ul FACS buffer. Cells were acquired on a Guava EasyCyte HT flow cytometer (Millipore) and analyzed with FlowJo software (FlowJo LLC) to determine the percentage of cells demonstrating phosphorylated STATS. Results were graphed in Prism software (GraphPad). P3 and P4 demonstrated little to no pSTAT5 signaling (FIG. 1B).

### Example 4

### Polypeptides of the Present Invention (P3, P4) Competitively Inhibited Binding of Neo-2/15 to the Human IL-2 Gamma Receptor

Binding to hIL2GB or mIL2GB was measured for Neo-2/15 in the presence of different concentrations of P3 and P4. hIL2RG or mIL2RG was immobilized in the surface of anti-human IgG Fc biosensors (ForteBio), for this purpose, sensors were soaked in samples containing 20 nM of hIL2RG-fc chimera (Symansis) for 300 sec. Subsequently, sensors were dipped in in twofold serial dilutions of P3 or P4 (200 uM-6.1 uM)+20 nM of Neo215+50 nM hIL2RB-fc chimera (Symansis) , and binding was measured for 600 sec. All samples were prepared using HBS-EP+buffer from GE Healthcare, which contains 10 mM HEPES, 150 mM NaCl, 3 mM EDTA and 0.05% v/v surfactant P20, pH 7.4. Data were acquired at 30° C. using an Octet RED96e system (ForteBio) and processed using the instrument's integrated software. IC50 values were estimated from response vs antagonist concentration plots. The binding of Neo-2/15 to human IL-2 receptor gamma and beta was inhibited by both P3 and P4. The binding of Neo-2/15 to mouse IL-2 receptor gamma and beta was inhibited by both P3 and P4, but to a lesser degree. IC50 values are shown in Table 3.

TABLE 3

Binding Inhibition
IC₅₀ (nM)

| Receptor -agonist | P3 | P4 |
|---|---|---|
| hIL2RBG - 20 nM Neo-2/15 | 12.5 | 0.9 |
| mIL2RBG - 20 nM Neo-2/15 | >200 | 67.6 |

### Example 5

### Polypeptides of the Present Invention (P3, P4) Inhibited Human IL-2 pSTAT5 Signaling on Human Pan T Cells

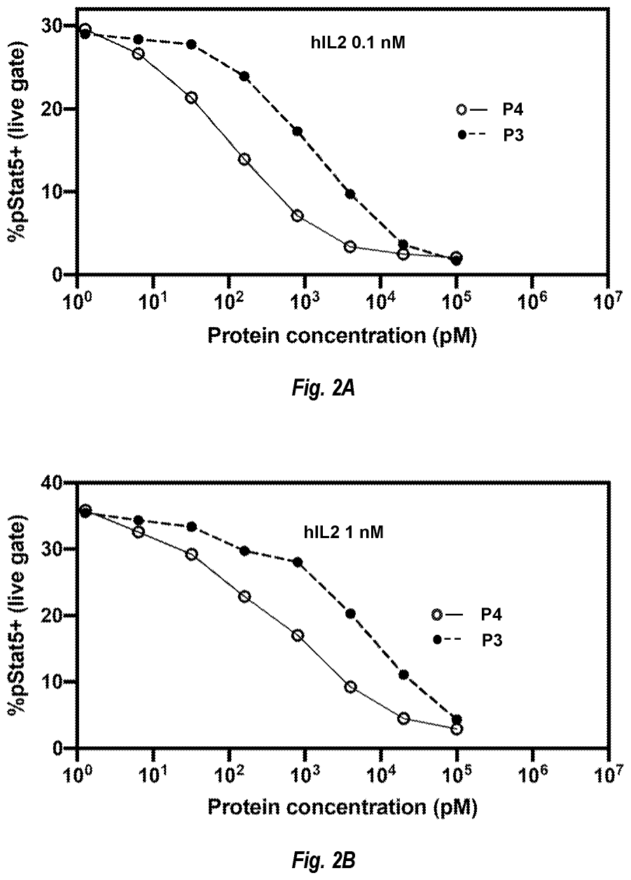

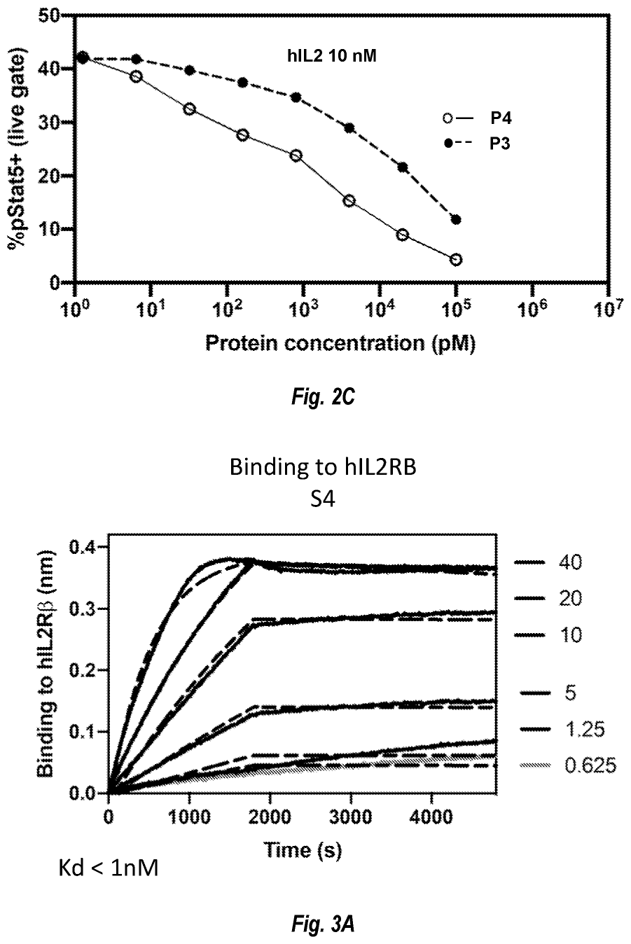

Human pan T cells were purified from PBMC and frozen for later use. Cells were thawed and rested overnight by culturing in X-VIVO 15 media (Lonza) without human IL-2. The following day, the T cells were enumerated and plated at 50,000 cells per well in 96 well plates. P3 and P4 were added to T cells starting at final concentration of 100 nM, titrating 8 points with 1:5 dilutions to roughly 1 pM. Recombinant human IL-2 (R&D Systems) was then added to cultures at a final concentration of 10 nM. Cultures were incubated at 37 degrees Celsius for 20 minutes before fixing cells in wells by adding formaldehyde to a final concentration of 1.5% by volume. Cells were fixed for 10 minutes, centrifuged and resuspended in 200 ul ice cold methanol to permeabilize the cells. Methanol was washed out twice with FACS buffer before adding anti-human pSTAT5 pY694 (BD Biosciences) diluted 1:25 in FACS buffer. Cells were incubated with antibody for 1 hour before washing twice and resuspending in 150 ul FACS buffer Cells were acquired on a Guava EasyCyte HT flow cytometer (Millipore) and analyzed with FlowJo Software (FlowJo LLC) to determine the percentage of cells demonstrating phosphorylated STATS. P3 and P4 proteins inhibited human IL-2 pSTAT5 at shown concentrations (FIGS. 2A-C). IC50 values are shown in Table 4

TABLE 4

| | |
|---|---|
| hIL2 | Binding Inhibition |
| concentration | IC₅₀ (nM) |

| (nM) | P3 | P4 |
|---|---|---|
| 0.1 | 1.4 | 0.1 |
| 1 | 11.7 | 0.45 |
| 10 | 863 | 3.3 |

### Example 6

### Polypeptides of the Present Invention (P4, P5, P6) Demonstrated High Binding Affinity to hIL-2RB, No Detectable Binding to hIL-2RG, and Inhibited Binding of hIL-2 to hIL-2RBG

Binding of P4, P5, and P6 to human IL2 receptor beta (hIL2RB) was analyzed by biolayer interferometry. Biotinylated hIL2RB molecules were immobilized to Streptavidin sensors (SA, ForteBio) at 2μg/mL in binding buffer (10 mM HEPES, pH 7.4, 150 mM

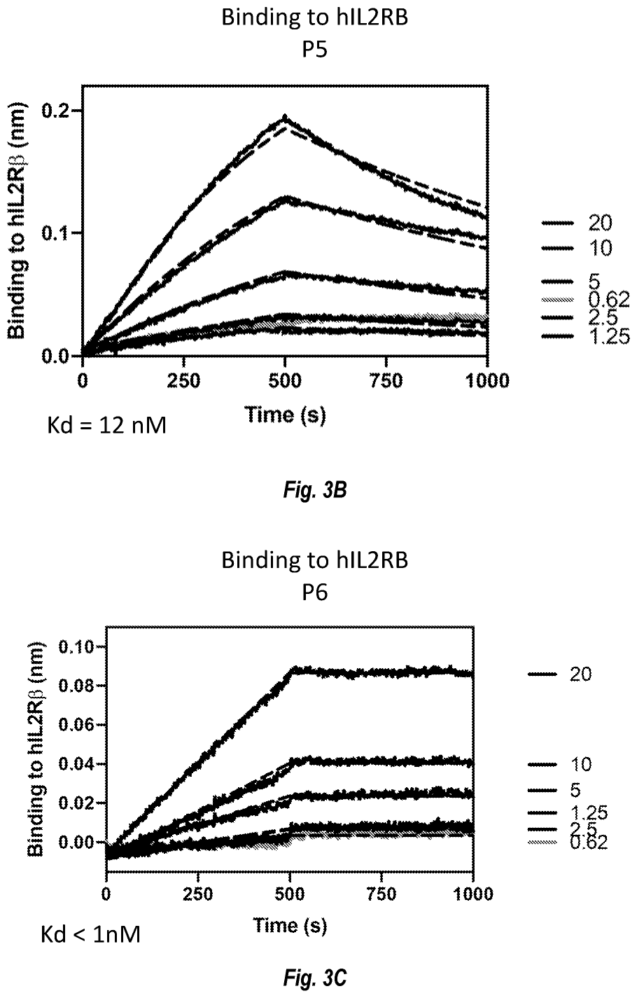

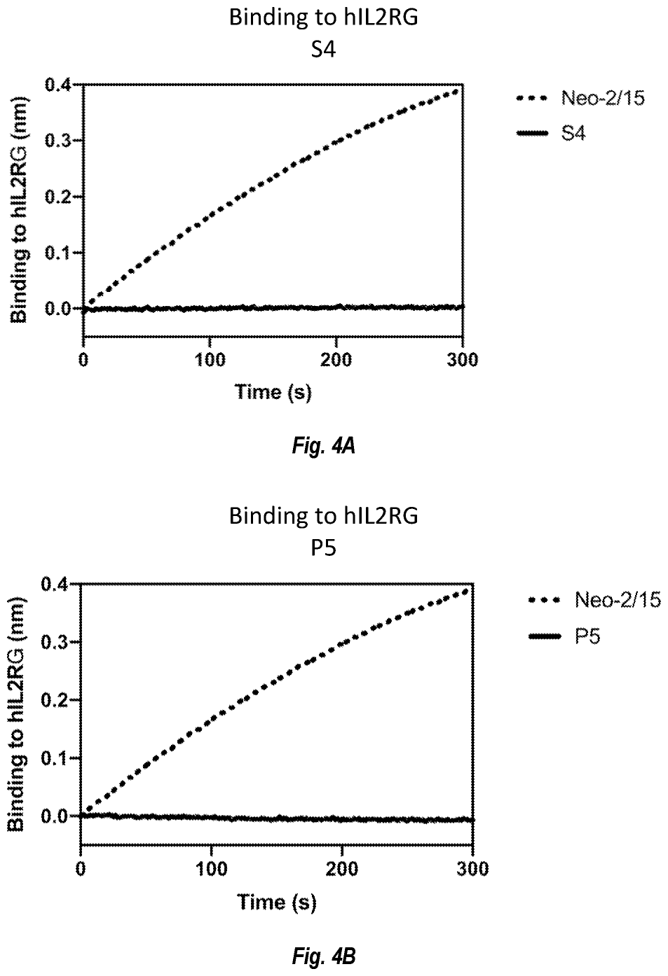

measurement in the binding buffer alone, the binding kinetics were monitored by dipping the biosensors in wells containing two-fold dilutions of the corresponding protein (20 to 0.62 nM) for 500sec (association) and then dipping the sensors back into baseline wells for 500 sec (dissociation). Data were collected in an Octet RED96 (ForteBio) and processed using the instrument's integrated software; kinetics were globally fit using a 1:1 binding to calculate the reported KD values. See FIGS. 3A-C

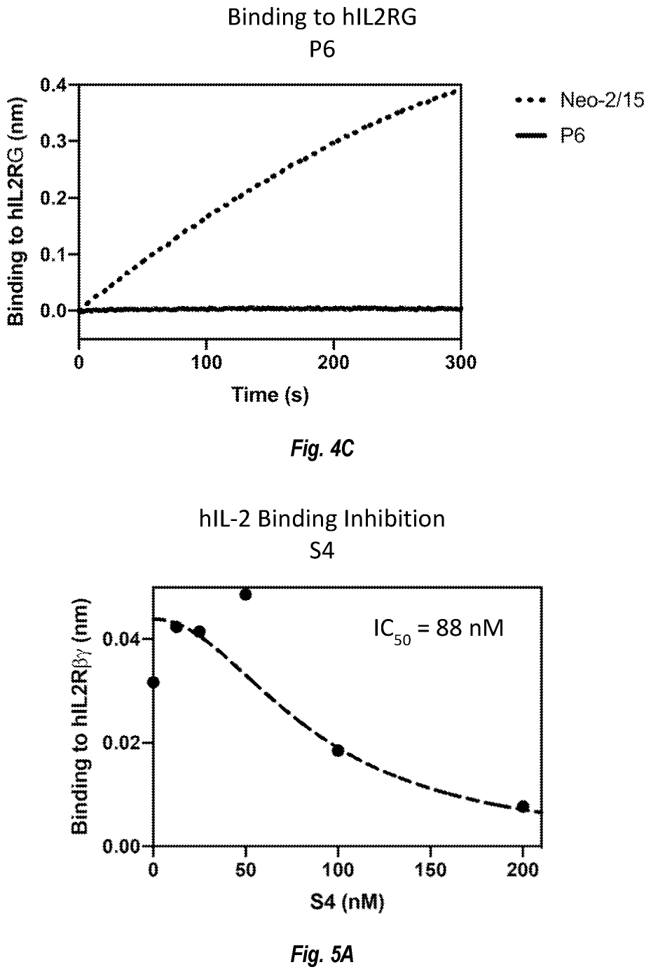

Binding of P4, P5, and P6 to human common gamma receptor (hIL2RG) was analyzed by biolayer interferometry. Biotinylated hIL2RG molecules were immobilized to Streptavidin sensors (SA, ForteBio) at 2 μg/mL in binding buffer (10 mM HEPES, pH 7.4, 150 mM NaCl, 3 mM EDTA, 0.05% surfactant P20, 0.5% non-fat dry milk). After baseline measurement in the binding buffer alone, the binding kinetics were monitored by dipping the biosensors in wells containing 200 nM of protein and 200 nM of hIL2RB for 300 sec. Data were collected in an Octet RED96 (ForteBio) and processed using the instrument's integrated software. See FIGS. 4A-C

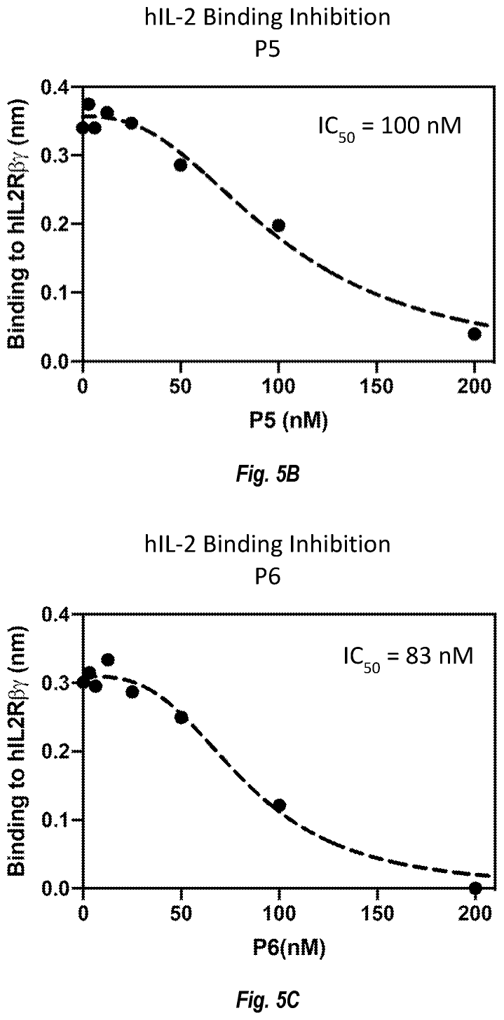

hIL2 binding inhibition assays were performed by biolayer interferometry. Biotinylated hIL2RG molecules were immobilized to Streptavidin sensors (SA, ForteBio) at 2 μg/mL in binding buffer (10 mM HEPES, pH 7.4, 150 mM NaCl, 3 mM EDTA, 0.05% surfactant P20, 0.5% non-fat dry milk). After baseline measurement in the binding buffer alone, the binding kinetics were monitored by dipping the biosensors in wells containing 50 nM of hIL2, 200 nM of hIL2RB and increasing concentrations of antagonist (0 to 200 nM). Data were collected in an Octet RED96 (ForteBio) and processed using the instrument's integrated software. Binding kinetics were fit using a one-association model to calculate the signal values once the equilibrium is reached (Req). The plot of Req vs the antagonist concentration was fit using a one-site binding model to calculate the IC50. See FIGS. 5A-C.

### Example 7

### Polypeptides of the Present Invention (P4, P5, P6) Inhibit IL-2R Signaling in T Cells

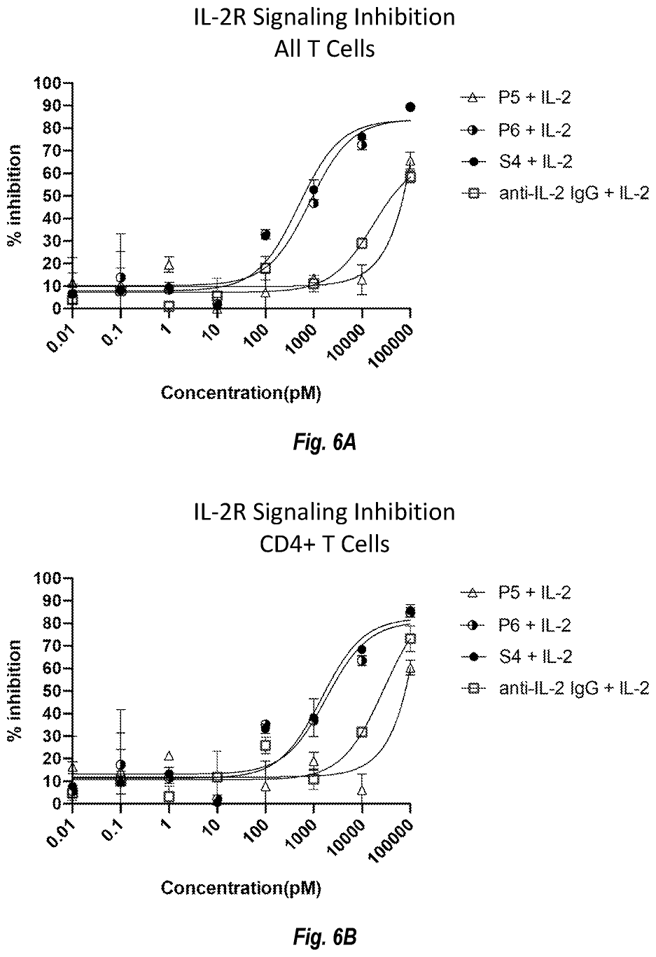

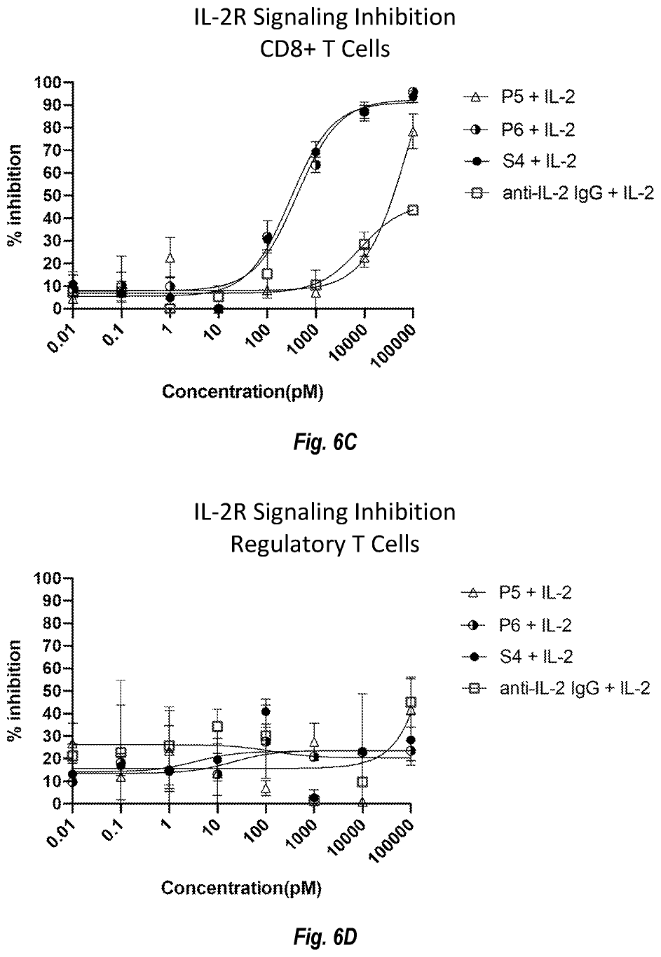

Human pan T cells were purified from whole blood and rested overnight by culturing in X-VIVO 15 media (Lonza) without human IL-2. The following day, the T cells were enumerated and plated at 100,000 cells per well in 96 well plates. Anti-IL-2 IgG, P5, P6, and S4 were added to T cells starting at final concentration of 100 nM, titrating 8 points with 1:10 dilutions to 0.01pM and incubated at 37C for 30 minutes. Recombinant human IL-2 (R&D Systems) was then added to cultures at a final concentration of 1 nM. Cultures were incubated at 37 degrees Celsius for 30 minutes before fixing cells in wells by adding formaldehyde to a final concentration of 1.5% by volume. Cells were fixed for 10 minutes at room temperature, centrifuged, and resuspended in 200 ul ice cold methanol to permeabilize the cells. Methanol was washed out twice with FACS buffer before adding antibodies (BD Biosciences) for anti-human pSTAT5 pY694 diluted 1:20, anti-human CD4 L200 diluted 1:50, anti-human CD8a SK1 diluted 1:20, anti-human CD25 M-A251 diluted 1:20, and anti-human CD127 HIL-7RM21 diluted 1:10 in FACS buffer. Cells were incubated with antibody for 1 hour before washing twice and resuspending in 100 ul FACS buffer. Cells were acquired on a Cytek Aurora flow cytometer (Cytek Biosciences) and analyzed with FlowJo Software (FlowJo LLC) to determine the percentage of cells demonstrating phosphorylated STATS. Live cells were gated on FSC and SSC plots. CD4⁺ cells and CD8⁺ cells were gated on live cells, and regulatory T cells were defined as the CD25+CD^{low} subset of CD4⁺ cells. Anti-IL-2 IgG, P5, P6, and S4 proteins inhibited human IL-2 pSTAT5 signaling at shown concentrations (FIGS. 6A-D).

Polypeptides of the invention (P4, P5 and P6) were not as effective at inhibiting murine IL-2R signaling (data not shown). This is believed to be due to weaker binding to the mouse IL-2R as compared to hIL-2R. S4 was determined to have a Kd of 70.6 nm to the mouse IL-2 beta receptor.

TABLE 5

Signaling Inhibition
IC₅₀ (nM)

| T Cell Type | S4 | P5 | P6 | Anti-IL-2 IgG |
|---|---|---|---|---|
| CD8+ | 0.3 | 77.73 | 0.41 | 8.3 |
| CD4+ | 1.46 | ND | 1.84 | 28.3 |
| All T Cells | 0.49 | ND | 0.83 | 17 |

### Example 8

### Polypeptides of the Present Invention (P4, P5, P6) Demonstrate Little to No pSTAT5 Signaling in T Cells

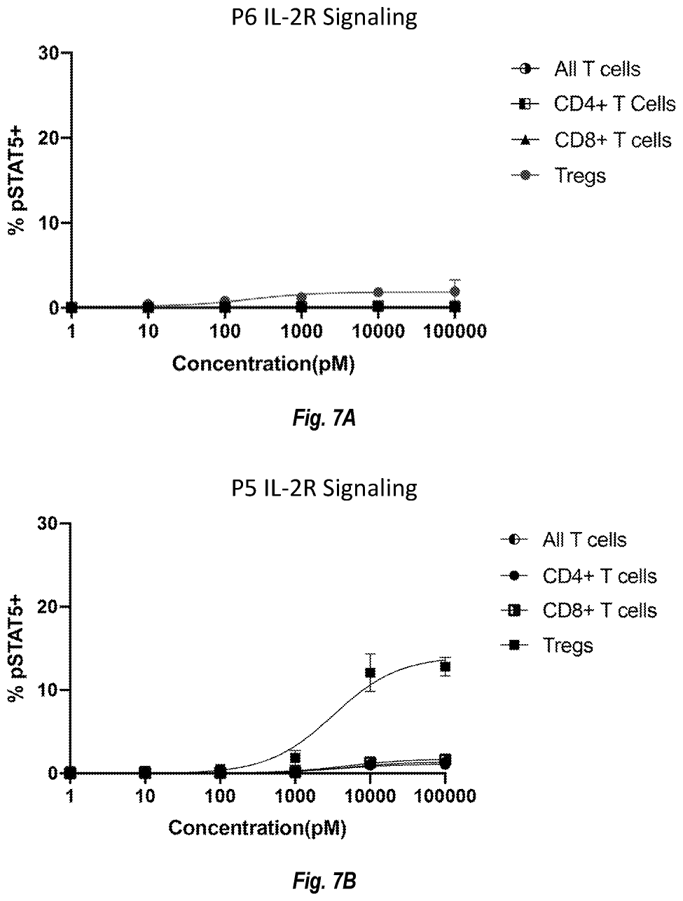

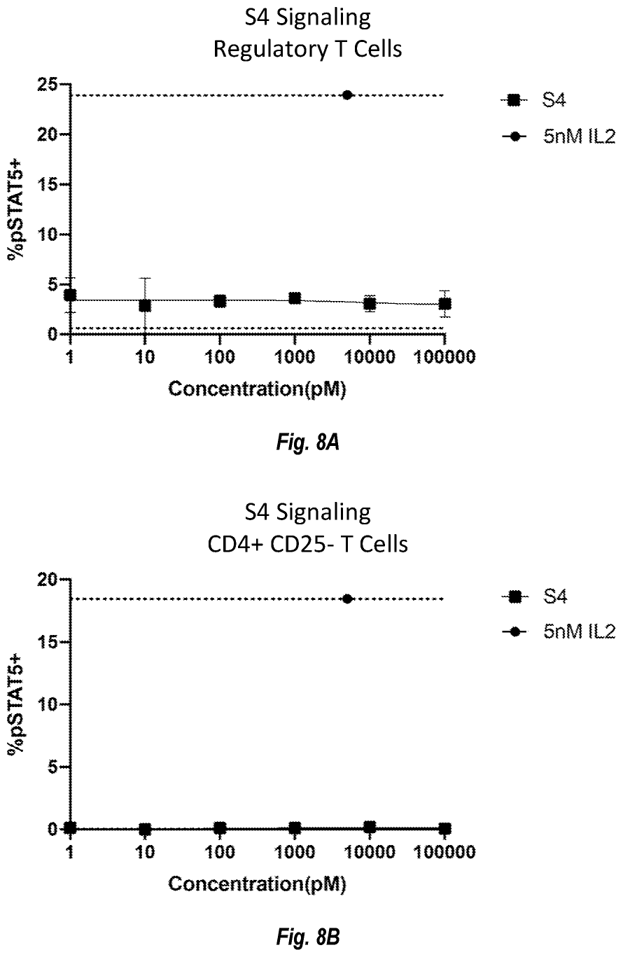

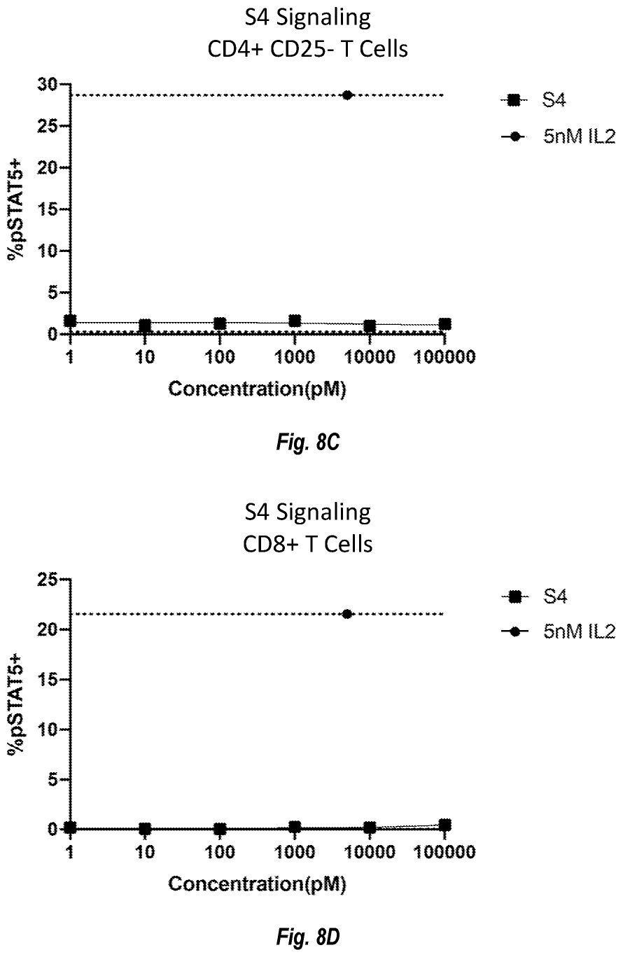

Human pan T cells were purified from whole blood and rested overnight by culturing in X-VIVO 15 media (Lonza) without human IL-2. The following day, T cells were enumerated and plated at 100,000 cells per well in 96 well plates. P5, P6, and S4 were added to T cells starting at final concentration of 100nM, titrating 6 points with 1:10 dilutions to 1pM and incubated at 37C for 30 minutes. Cells were fixed in wells by adding formaldehyde to a final concentration of 1.5% by volume for 10 minutes at room temperature, then centrifuged and resuspended in 200 ul ice cold methanol to permeabilize the cells. Methanol was washed out twice with FACS buffer before adding antibodies (BD Biosciences) for anti-human pSTAT5 pY694 diluted 1:20, anti-human CD4 L200 diluted 1:50, anti-human CD8a SK1 diluted 1:20, anti-human CD25 M-A251 diluted 1:20, and anti-human CD127 HIL-7RM21 diluted 1:10 in FACS buffer. Cells were incubated with antibody for 1 hour before washing twice and resuspending in 100 uL FACS buffer. Cells were acquired on a Cytek Aurora flow cytometer (Cytek Biosciences) and analyzed with FlowJo Software (FlowJo LLC) to determine the percentage of cells demonstrating phosphorylated STATS. Live cells were gated on FSC and SSC plots. CD4⁺ cells and CD8⁺ cells were gated on live cells, and regulatory T cells were defined as the CD25⁺ CD1271^{low} subset of CD⁺ cells. P5, P6, and S4 proteins showed negligible pSTAT5 signaling at shown concentrations (FIGS. 7A-B, 8A-D).

### Example 9

### Polypeptides of the Present Invention (P4) Demonstrate Little to No pSTAT5 Signaling in PBMC and NK Cells

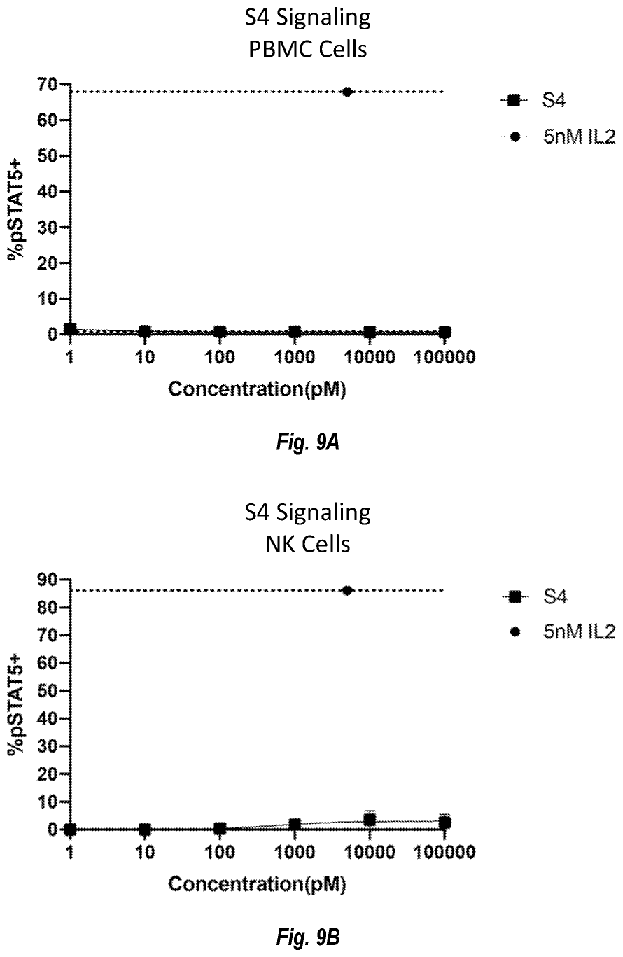

Human PBMCs or NK cells were purified from whole blood and rested overnight by culturing in X-VIVO 15 media (Lonza) without human IL-2. The following day, PBMCs were enumerated and plated at 200,000 cells per well in 96-well plates. NK cells were enumerated and plated at 50,000 cells per well in 96-well plates. S4 was added to cells starting at final concentration of 100nM, titrating 6 points with 1:10 dilutions to 1pM and incubated at 37C for 30 minutes. Cells were fixed in wells by adding formaldehyde to a final concentration of 1.5% by volume for 10 minutes at room temperature, then centrifuged and resuspended in 200 ul ice cold methanol to permeabilize the cells. Methanol was washed out twice with FACS buffer before adding antibody for anti-human pSTAT5 pY694 (BD Biosciences) diluted 1:20 in FACS buffer. Cells were incubated with antibody for 1 hour before washing twice and resuspending in 100 uL FACS buffer. Cells were acquired on a Cytek Aurora flow cytometer (Cytek Biosciences) and analyzed with FlowJo Software (FlowJo LLC) to determine the percentage of cells demonstrating phosphorylated STATS. Live cells were gated on SSC and FSC plots. S4 protein showed negligible pSTAT5 signaling in both PBMCs and NK cells at shown concentrations (FIG. 9A-B)

### Example 10

### Polypeptides of the Present Invention (S4) Inhibit IL-15 Signaling in T Cells

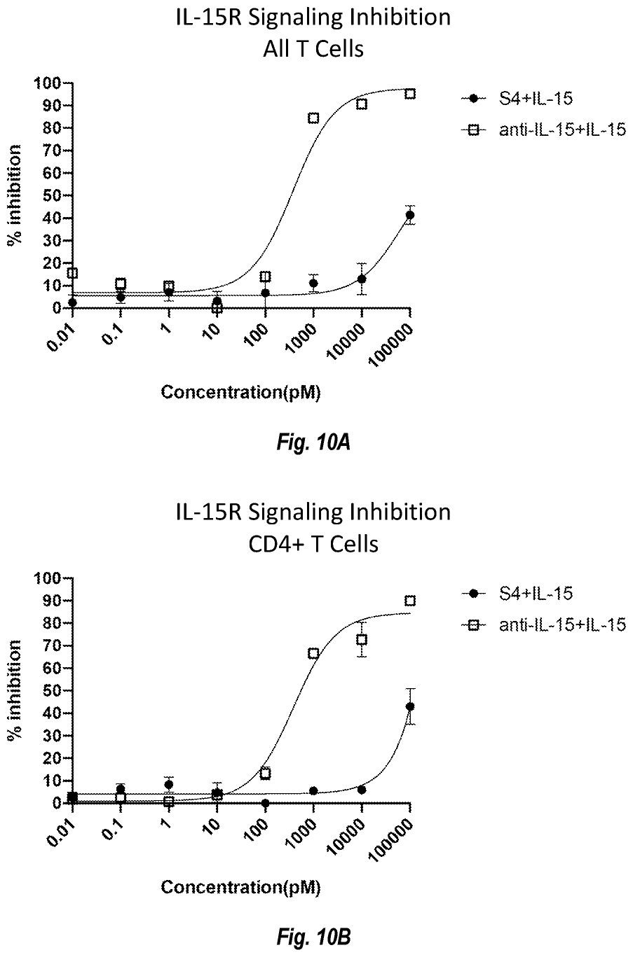

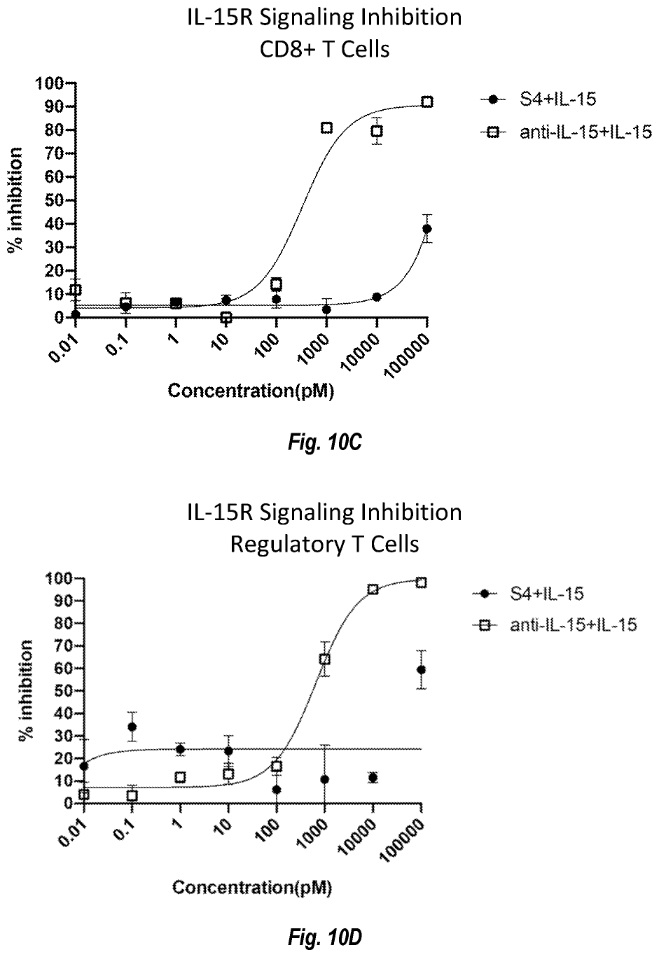

Human pan T cells were purified from whole blood and rested overnight by culturing in X-VIVO 15 media (Lonza) without human IL-2. The following day, the T cells were enumerated and plated at 100,000 cells per well in 96 well plates. Anti-IL-15 and S4 were added to T cells starting at final concentration of 100 nM, titrating 8 points with 1:10 dilutions to 0.01 pM and incubated at 37 C for 30 minutes. Recombinant human IL-15 (R&D Systems) was then added to cultures at a final concentration of 5 pM. Cultures were incubated at 37 degrees Celsius for 30 minutes before fixing cells in wells by adding formaldehyde to a final concentration of 1.5% by volume. Cells were fixed for 10 minutes at room temperature, centrifuged, and resuspended in 200 ul ice cold methanol to permeabilize the cells. Methanol was washed out twice with FACS buffer before adding antibodies (BD Biosciences) for anti-human pSTAT5 pY694 diluted 1:20, anti-human CD4 L200 diluted 1:50, anti-human CD8a SK1 diluted 1:20, anti-human CD25 M-A251 diluted 1:20, and anti-human CD127 HIL-7RM21 diluted 1:10 in FACS buffer. Cells were incubated with antibody for 1 hour before washing twice and resuspending in 100 ul FACS buffer. Cells were acquired on a Cytek Aurora flow cytometer (Cytek Biosciences) and analyzed with FlowJo Software (FlowJo LLC) to determine the percentage of cells demonstrating phosphorylated STATS. Live cells were gated on FSC and SSC plots. CD4⁺ cells and CD8⁺ cells were gated on live cells, and regulatory T cells were defined as the CD25⁺CD127^{low} subset of CD4⁺ cells. Anti-IL-2 IgG, P5, P6, and S4 proteins inhibited human IL-15 pSTAT5 signaling at shown concentrations (FIGS. 10A-D).

### Example 11

### Polypeptides of the Present Invention are Hyperstable

Far-ultraviolet circular dichroism measurements were carried out on S4, P5 and P6 using an CHIRASCAN spectrometer V100 (Applied Photophysics). Protein Samples were measured in PBS buffer (pH 7.4) at protein concentrations of 0.2 mg/mL, using a 0.1 mm path-length cuvette. Temperature unfolding curves were obtained from 20 to 98° C. by monitoring the absorption signal at 222 nm (steps of 0.5° C. per min, 30 s of equilibration by step). Wavelength scans (200-250 nm) were collected at 20° C., 98° C., and again at 20° C. after refolding.

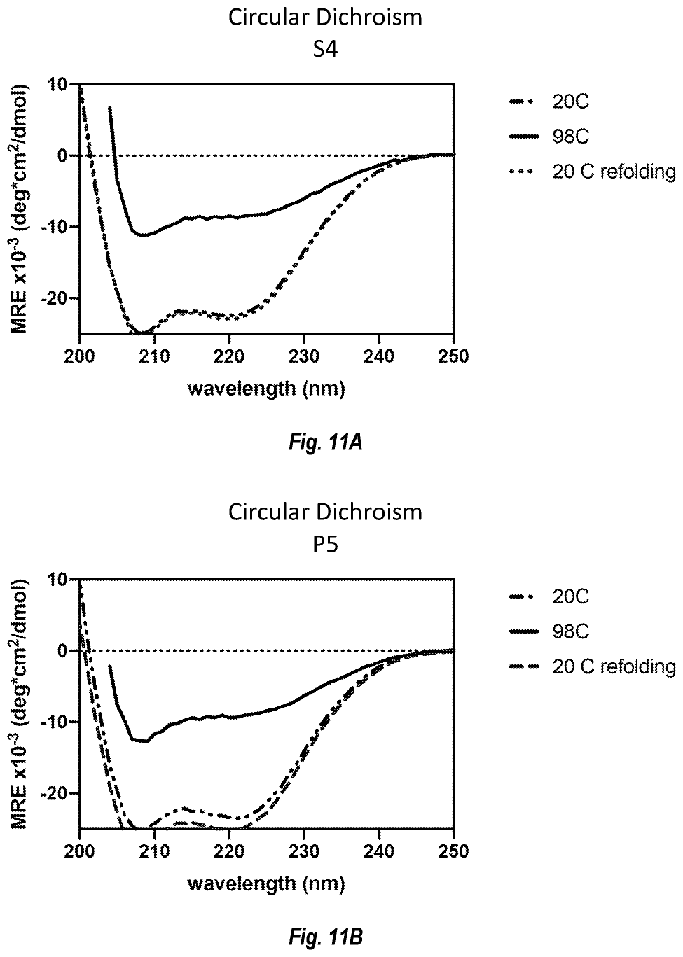

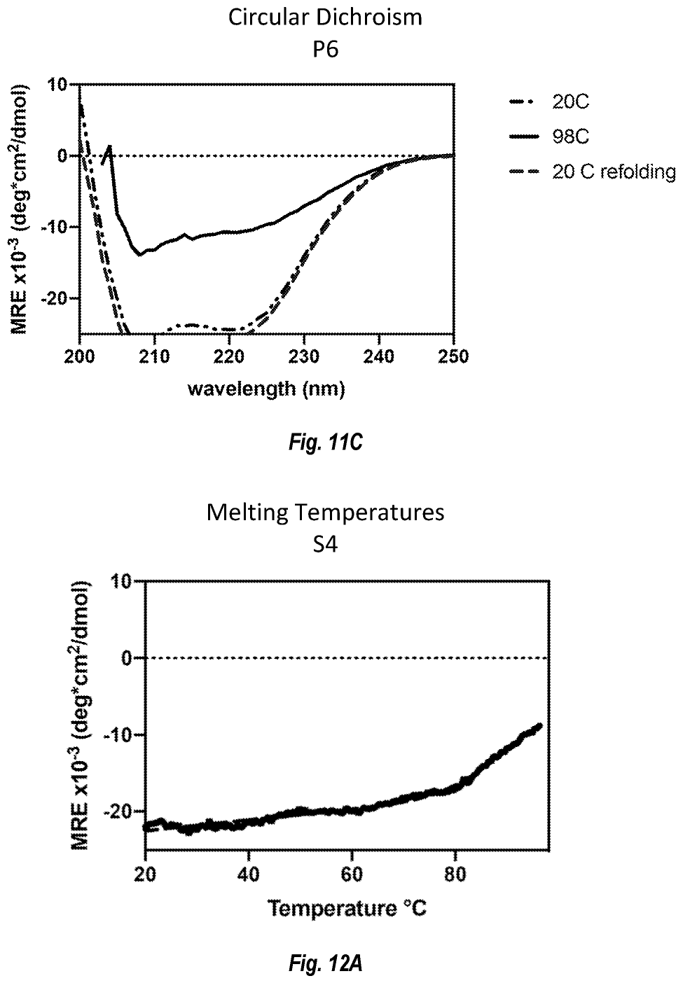

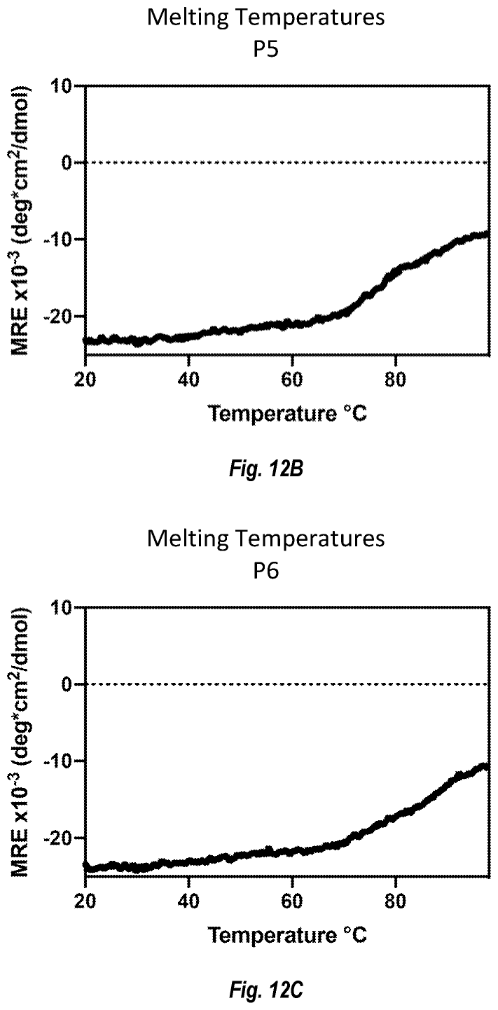

The shape and signal of the scans indicate that the proteins are structured at 20 C (two minima at 208 and 222 nm with MRE>=-20 deg cm2 dmol⁻¹), unfold and 98 C and refold after decreasing the temperature to the initial value. The unfolding curves were fit using a Van't Hoff equation-based model to calculate the melting temperatures (Tm). The Tm values obtained for all proteins are similar or higher than those calculated for thermophilic proteins (˜80 C), indicating that they are folded even at high temperatures. See FIGS. 11A-C and 12A-C

### Example 12

### Polypeptides of the Present Invention are Tolerant to Amino Acid Substitutions

As taught in the present application, amino acid substitutions can be tolerated at multiple positions. S4 variants with mutations at select positions were made and tested for binding to IL-2β. Positions that were mutated include: I22F, P29L, A30V, K3E, and S54G, position numbering in accordance with SEQ ID NO:9. All variants retained beta binding activity (data not shown).

We claim:

1. A polypeptide that binds IL-2 receptor beta (IL-2Rβ) and comprises the domains D1, D2, D3, and D4 wherein:
D1 comprises the amino acid sequence: KIQLYAEHAL YDAX₁₇MILX₂₁I (SEQ ID NO: 1)
D2 comprises an amino acid sequence at least 8 amino acids in length;
D3 comprises the amino acid sequence ELEDYAFN FELILEEIAR LFESG (SEQ ID NO:2)
D4 comprises the amino acid sequence EDEQEEMANX₈₉I X₉₁X₉₂ILX₉₅X₉₆WIX₉₉S (SEQ ID NO:3)
wherein:
(i) D1, D2, D3 and D4 may be in any order in the polypeptide;
(ii) amino acid linkers may be present between any of the domains,
(iii) Xi7 is glutamic acid or aspartic acid; X₂₁ is any amino acid; X₈₉ is arginine or lysine; X₉₁ is arginine or lysine; X₉₂ is arginine or lysine; X₉₅ is threonine, serine, glutamic acid, or aspartic acid; X₉₆ is aspartic acid or glutamic acid; and X₉₉ is arginine or lysine; and
(iv) wherein the polypeptide contains a total of no more than ten, no more than nine, no more than eight, no more than seven, no more than six, no more than five, no more than four, no more than three, or no more than two substitutions, at amino acid positions not designated as X.

2. The polypeptide of claim 1, wherein:
if there is a substitution of the tyrosine at position 5 of D1, it is a substitution to phenylalanine, wherein the position numbering of D1 is according to SEQ ID NO:l.

3. The polypeptide of claim 1, wherein:
if there is a substitution of the tyrosine at position 5 of D1, it is a substitution to histidine, wherein the position numbering of D1 is according to SEQ ID NO:1.

4. The polypeptide of any one of claims 1-3, wherein:
if there is a substitution of the glutamic acid at position 1 of D3, it is a substitution to cysteine, aspartic acid, or tyrosine, wherein the position numbering of D3 is according to SEQ ID NO:2.

5. The polypeptide of any one of claims 1-3, wherein:
if there is a substitution of the glutamic acid at position 1 of D3, it is a substitution to lysine, wherein the position numbering of D3 is according to SEQ ID NO:2.

6. A polypeptide that binds IL-2 receptor beta (IL-2Rβ) and comprises the domains D1, D2, D3 and D4 wherein:
D1 comprises the amino acid sequence: KIQLX₈AEHAL YDAX₁₇MILX₂₁I (SEQ ID NO: 4)
D2 comprises an amino acid sequence at least 8 amino acids in length;
D3 comprises the amino acid sequence X₃₃LEDYAFN FELILEEIAR LFESG (SEQ ID NO:5)
D4 comprises the amino acid sequence. EDEQEEMANX₈₉I X₉₁X₉₂₁LX₉₅X₉₆W1X₉₉S (SEQ ID NO:3)
wherein:
(i) D1, D2, D3 and D4 may be in any order in the polypeptide;
(ii) amino acid linkers may be present between any of the domains, X₈ is any amino acid; X₁₇ is glutamic acid or aspartic acid; X₂i is any amino acid; X₃₃ is any amino acid; X₈₉ is arginine or lysine; X₉₁ is arginine or lysine;
X₉₂ is arginine or lysine; X₉₅ is threonine, serine, glutamic acid, or aspartic acid; X₉₆ is aspartic acid or glutamic acid; and X₉₉ is arginine or lysine; and
(iii) wherein the polypeptide contains a total of no more than ten, no more than nine, no more than eight, no more than seven, no more than six, no more than five, no more than four, no more than three, or no more than two substitutions, at amino acid positions not designated as X.

7. The polyeptide of claim 6 wherein:
X₈ is alanine, asparagine, aspartic acid, arginine, cysteine, glutamic acid, glutamine, glycine, histidine, isoleucine, leucine, lysine, methionine, phenylalanine, serine, threonine, tryptophan, tyrosine, or valine.

8. The polypeptide of claim 7 wherein:
X₈ is histidine, tyrosine or phenylalanine.

9. The polypeptide of claim 7 wherein:
X₈ is tyrosine or phenylalanine.

10. The polypeptide of any one of claims 6-9, wherein:
X₃₃ is cysteine, tyrosine, lysine, glutamic acid or aspartic acid.

11. The polypeptide of claim 10, wherein:
X₃₃ is cysteine, tyrosine, glutamic acid or aspartic acid.

12. The polypeptide of claim 10, wherein:
X₃₃ is glutamic acid or aspartic acid.

13. The polypeptide of claim 12, wherein:
X₃₃ is glutamic acid.

14. The polypeptide of any one of claims 1 to 13 wherein:
X₁₇ is glutamic acid;
X₉₁ is arginine;
X₉₂ is lysine;
X₉₆ is glutamic acid; and
X₉₉ is arginine.

15. The polypeptide of any one of claims 1 to 14 wherein:
X₉₅ is threonine, glutamic acid, or aspartic acid.

16. The polypeptide of claim 15 wherein:
X₉₅ is threonine or glutamic acid.

17. The polypeptide of claim 16 wherein:
X₉₅ is glutamic acid.

18. The polypeptide of any one of claims 1 to 17, wherein:
X₂i is alanine, asparagine, aspartic acid, arginine, cysteine, glutamic acid, glutamine, glycine, histidine, isoleucine, leucine, lysine, methionine, proline, serine, threonine, tryptophan, tyrosine, or valine.

19. The polypeptide of claim 18, wherein:
X₂₁ is asparagine or lysine.

20. The polypeptide of claim 19, wherein:
X₂₁ is lysine.

21. A polypeptide that binds IL-2 receptor beta (IL-2Rβ) and comprises the domains D1, D2, D3, and D4 wherein:
D1 comprises the amino acid sequence: KIQLFAEHAL YDAX₁₇MILKI (SEQ ID NO: 21)
D2 comprises an amino acid sequence at least 8 amino acids in length;
D3 comprises the amino acid sequence ELEDYAFN FELILEEIAR LFESG (SEQ ID NO:2)
D4 comprises the amino acid sequence EDEQEEMANKI RKILX₉₅EWIX₉S (SEQ ID NO: 29)
wherein:
(i) D1, D2, D3 and D4 may be in any order in the polypeptide;
(ii) amino acid linkers may be present between any of the domains,
(iii) X₁₇ is glutamic acid or aspartic acid; X₉₅ is threonine, serine, glutamic acid, or aspartic acid; and X₉₉ is arginine or lysine; and
(iv) wherein the polypeptide contains a total of no more than ten, no more than nine, no more than eight, no more than seven, no more than six, no more than five, no more than four, no more than three, or no more than two substitutions, at amino acid positions not designated as X.

22. A polypeptide that binds IL-2 receptor beta (IL-2Rβ) and comprises the domains D1, D2, D3, and D4 wherein:
D1 comprises the amino acid sequence: KIQLYAEHAL YDAX₁₇MILKI (SEQ ID NO: 32)
D2 comprises an amino acid sequence at least 8 amino acids in length;
D3 comprises the amino acid sequence ELEDYAFN FELILEEIAR LFESG (SEQ ID NO:2)
D4 comprises the amino acid sequence EDEQEEMANRI RKILX₉₅EWIX₉₉S (SEQ ID NO: 47)
wherein:
(i) D1, D2, D3 and D4 may be in any order in the polypeptide;
(ii) amino acid linkers may be present between any of the domains,
(iii) Xi7 is glutamic acid or aspartic acid; X₉₅ is threonine, serine, glutamic acid, or aspartic acid; and X₉₉ is arginine or lysine; and
(iv) wherein the polypeptide contains a total of no more than ten, no more than nine, no more than eight, no more than seven, no more than six, no more than five, no more than four, no more than three, or no more than two substitutions, at amino acid positions not designated as X.

23. The polypeptide of claim 21 or 22, wherein X₁₇ is glutamic acid and X₉₉ is arginine.

24. A polypeptide that binds IL-2 receptor beta (IL-2Rβ) and comprises the domains D1, D2, D3, and D4 wherein:
D1 comprises the amino acid sequence: KIQLFAEHAL YDAEMILKI (SEQ ID NO: 27)
D2 comprises an amino acid sequence at least 8 amino acids in length;
D3 comprises the amino acid sequence ELEDYAFN FELILEEIAR LFESG (SEQ ID NO:2)
D4 comprises the amino acid sequence EDEQEEMANKI RKILX₉₅EWIX₉₉S (SEQ ID NO: 29)
wherein:
(i) D1, D2, D3 and D4 may be in any order in the polypeptide;
(ii) amino acid linkers may be present between any of the domains,
(iii) X₉₅ is threonine, serine, glutamic acid, or aspartic acid; and X₉₉ is arginine or lysine; and
(iv) wherein the polypeptide contains a total of no more than ten, no more than nine, no more than eight, no more than seven, no more than six, no more than five, no more than four, no more than three, or no more than two substitutions, at amino acid positions not designated as X.

25. A polypeptide that binds IL-2 receptor beta (IL-2Rβ) and comprises the domains D1, D2, D3, and D4 wherein:
D1 comprises the amino acid sequence: KIQLYAEHAL YDAEMILKI (SEQ ID NO:44),
D2 comprises an amino acid sequence at least 8 amino acids in length;
D3 comprises the amino acid sequence ELEDYAFN FELILEEIAR LFESG (SEQ ID NO:2)
D4 comprises the amino acid sequence EDEQEEMANRI RKILX₉₅EWIX₉₉S (SEQ ID NO: 47)
wherein:
(i) D1, D2, D3 and D4 may be in any order in the polypeptide;
(ii) amino acid linkers may be present between any of the domains,
(iii) X₉₅ is threonine, serine, glutamic acid, or aspartic acid; and X₉₉ is arginine or lysine; and
(iv) wherein the polypeptide contains a total of no more than ten, no more than nine, no more than eight, no more than seven, no more than six, no more than five, no more than four, no more than three, or no more than two substitutions, at amino acid positions not designated as X.

26. The polypeptide of any one of claims 21-25 wherein X₉₉ is arginine.

27. The polypeptide of any one of claims claim 21-26, wherein X₉₅ is threonine or glutamic acid.

28. The polypeptide of any one of claims 21-27 wherein one, two, three, or four of the following are true: if there is a substitution at position 10 of D4, it is to arginine or lysine; if there is a substitution at position 12 of D4 it is to lysine; if there is a substitution at position 13 of D4, it is to arginine; and/or if there is a substitution at position 17 of D4, it is to aspartic acid, wherein the position numbering of D4 is according to SEQ ID NO: 29 or 47.

29. The polypeptide of any one of claims 21-27 wherein if there is a substitution at position 12 of D4 it is to lysine; if there is a substitution at position 13 of D4, it is to arginine; and/or if there is a substitution at position 17 of D4, it is to aspartic acid, wherein the position numbering of D4 is according to SEQ ID NO: 29 or 47.

30. The polypeptide of any one of claims 21-27, wherein there are no substitutions at positions 12, 13, and 17 of D4 wherein the position numbering of D4 is according to SEQ ID NO: 29 or 47.

31. The polypeptide of any one of claims 21-30, wherein there are no substitutions at position 10 of D4 wherein the position numbering of D4 is according to SEQ ID NO: 29 or 47.

32. The polypeptide of any one of claims 24-31 wherein if there is a substitution at the glutamic acid of position 14 of D1, it is to aspartic acid, wherein the position numbering of D1 is according to SEQ ID NO: 27 or 44.

33. A polypeptide that binds IL-2 receptor beta (IL-2Rβ) and comprises the domains D1, D2, D3, and D4 wherein:
D1 comprises the amino acid sequence: KIQLYAEHAX₁₃ YDAX₁₇MILNI (SEQ ID NO: 20)
D2 comprises an amino acid sequence at least 8 amino acids in length;
D3 comprises the amino acid sequence ELEDYAFN FELILEEIAR LFESG (SEQ ID NO:2)
D4 comprises the amino acid sequence EDEQEEMANAI ITILX₉₅SWIX₉₉S (SEQ ID NO:22)
wherein:
(i) D1, D2, D3 and D4 may be in any order in the polypeptide;
(ii) amino acid linkers may be present between any of the domains,
(iii) Xi3 is arginine or lysine; X₁₇ is glutamic acid or aspartic acid; X₉₅ is threonine, serine, glutamic acid, or aspartic acid; and X₉₉ is arginine or lysine; and
(iv) wherein the polypeptide contains a total of no more than ten, no more than nine, no more than eight, no more than seven, no more than six, no more than five, no more than four, no more than three, or no more than two substitutions, at amino acid positions not designated as X.

34. The polypeptide of claim 33 wherein X₁₃ is arginine, X₁₇ is glutamic acid, and X₉₉ is arginine.

35. The polypeptide of claim 33 or 34 wherein X₉₅ is threonine.

36. A polypeptide that binds IL-2 receptor beta (IL-2Rβ) and comprises the domains D1, D2, D3 and D4 wherein:
D1comprises an amino acid sequence at least 80% identical to the amino acid sequence: KIQLFAEHAL YDAEMILKI (SEQ ID NO: 27)
D2 comprises an amino acid sequence at least 8 amino acids in length;
D3 comprises an amino acid sequence at least 80% identical to the amino acid sequence ELEDYAFN FELILEEIAR LFESG (SEQ ID NO:2)
D4 comprises an amino acid sequence at least 80% identical to the amino acid sequence EDEQEEMANKI RKILEEWIRS (SEQ ID NO: 43) wherein D1, D2, D3 and D4 may be in any order in the polypeptide; and amino acid linkers may be present between any of the domains; and wherein the polypeptide comprises, a threonine, serine, glutamic acid or aspartic acid at position 16 of D4, and an arginine or lysine at position 20 of D4, wherein the position numbering of D4 is according to SEQ ID NO: 43.

37. The polypeptide of claim 36 wherein the polypeptide comprises a glutamic acid or aspartic acid at position 14 of D1 and an arginine at position 20 of D4, wherein the position numbering of D1 is according to SEQ ID NO:27.

38. The polypeptide of claim 35 wherein the polypeptide comprises a glutamic acid at position 14 of D1.

39. The polypeptide of any one of claims 36-38 wherein the polypeptide comprises a glutamic acid at position 16 of D4.

40. The polypeptide of any one of claims 36-39 wherein one, two, three, four, of the following are true: the polypeptide comprises an arginine or lysine at position 10 of D4, a lysine or arginine at position 12 of D4, an arginine or lysine at position 13 of D4, and a glutamic acid or aspartic acid at position 17 of D4.

41. The polypeptide of any one of claims 36-39 wherein the polypeptide comprises an arginine or lysine at position 10 of D4, an arginine at position 12 of D4, a lysine at position 13 of D4, and a glutamic acid at position 17 of D4.

42. The polypeptide of any one of claims 36-39 wherein the polypeptide comprises an arginine at position 12 of D4, a lysine at position 13 of D4, and a glutamic acid at position 17 of D4.

43. The polypeptide of any one of claims 36-42 wherein D3 comprises an amino acid sequence at least 90% identical to the amino acid sequence set forth in SEQ ID NO:2.

44. The polypeptide of any one of claims 36-43 wherein D4 comprises an amino acid sequence at least 90% identical to the amino acid sequence set forth in SEQ ID NO:43.

45. The polypeptide of any one of claims 1-44 wherein D2 is at least 19 amino acids in length.

46. The polypeptide of claim 45 wherein D2 comprises an amino acid sequence at least 84%, 89%, or 94% identical to the amino acid sequence KDEAEK AKRMKEWMKR IKT (SEQ ID NO: 6) .

47. The polypeptide of claim 45 wherein D2 comprises the amino acid sequence of SEQ ID NO:6.

48. The polypeptide of any one of claims 1-47 wherein the amino acid linkers are 1-100, 1-90, 1-80, 1-70, 1-60, 1-50, 1-40, 1-30, 1-20, 1-10, or 2-10 amino acids in length.

49. The polypeptide of any one of claims 1-48 wherein the order of the four domains is D1-D3-D2-D4.

50. A polypeptide that binds IL-2 receptor beta (IL-2Rβ) and comprises an amino acid sequence at least 80% identical to the amino acid sequence of SEQ ID NO: 7:
(SEQ ID NO: 7)
KIQLYAEHAL YDAEMILKIV KTNSPPAEEE LEDYAFNFEL
ILEEIARLFE SGDQKDEAEK AKRMKEWMKR IKTTASEDEQ
EEMANRIRKI LEEWIRS;
wherein the polypeptide comprises :
a glutamic acid, aspartic acid, threonine, or serine at position 95,
an arginine or lysine at position 99, and
one or more of, two or more of, three or more of, four or more of, or all five of:
a glutamic acid or aspartic acid at position 17;
an arginine or lysine at position 89
an arginine or lysine at position 91,
a lysine or arginine at position 92, and/or
a glutamic acid or aspartic acid at position 96
wherein the position numbering is according to SEQ ID NO: 7, provided that the lysine at the N terminus of SEQ ID NO: 7 is designated as position 4.

51. The polypeptide of claim 50, wherein the polypeptide comprises an amino acid sequence at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 7.

52. A polypeptide that binds IL-2 receptor beta (IL-2Rβ) and comprises an amino acid sequence at least 80% identical to the amino acid sequence of SEQ ID NO: 8:
(SEQ ID NO: 8)
KIQLFAEHAL YDAEMILKIV KTNSPPAEEE LEDYAFNFEL
ILEEIARLFE SGDQKDEAEK AKRMKEWMKR IKTTASEDEQ
EEMANKIRKI LEEWIRS;
wherein the polypeptide comprises :
a glutamic acid, aspartic acid, threonine, or serine at position 95,
an arginine or lysine at position 99, and
one or more of, two or more of, three or more of, four or more of, or all five of:
a glutamic acid or aspartic acid at position 17;
an arginine or lysine at position 89
an arginine or lysine at position 91,
a lysine or arginine at position 92, and/or
a glutamic acid or aspartic acid at position 96
wherein the position numbering is according to SEQ ID NO: 8, provided that the lysine at the N terminus of SEQ ID NO: 8 is designated as position 4.

53. The polypeptide of claim 52, wherein the polypeptide comprises an amino acid sequence at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 8.

54. The polypeptide of any one of claims 50 to 53, wherein the polypeptide comprises a glutamic acid or aspartic acid at position 17.

55. The polypeptide of claim 54, wherein the polypeptide comprises one or more of, two or more of, three or more of, or all four of (i) an arginine or lysine at position 89, (ii) an arginine or lysine at position 91, (iii) a lysine or arginine at position 92, and/or (iv) a glutamic acid or aspartic acid at position 96.

56. The polypeptide of any one of claims 50-55, wherein the polypeptide comprises an alanine, asparagine, aspartic acid, arginine, cysteine, glutamic acid, glutamine, glycine, histidine, isoleucine, leucine, lysine, methionine, proline, serine, threonine, tryptophan, tyrosine, or valine at position 21.

57. The polypeptide of claim 56, wherein the polypeptide comprises a lysine or arginine at position 21.

58. The polypeptide of claim 56, wherein the polypeptide comprises a lysine at position 21.

59. The polypeptide of any one of claims 50-58, wherein the polypeptide comprises one or more of, two or more of, or all three of an arginine at position 91, a lysine at position 92, and/or a glutamic acid at position 96.

60. The polypeptide of any one of claims 50-58, wherein the polypeptide comprises an arginine or lysine at position 89, an arginine or lysine at position 91, an arginine or lysine at position 92, and a glutamic acid or aspartic acid at position 96.

61. The polypeptide of claim 60, wherein the polypeptide comprises an arginine at position 91, a lysine at position 92, and a glutamic acid at position 96.

62. The polypeptide of any one of claims 50-61, wherein the polypeptide comprises an arginine at position 89.

63. The polypeptide of any one of claims 50-61, wherein the polypeptide comprises a lysine at position 89.

64. The polypeptide of any one of claims 50-63, wherein the polypeptide comprises a histidine at position 8.

65. The polypeptide of any one of claims 50-63, wherein the polypeptide comprises a tyrosine or phenylalanine at position 8.

66. The polypeptide of any one of claims 50-65, wherein the polypeptide comprises a cysteine, tyrosine, lysine, glutamic acid or aspartic acid at position 33.

67. The polypeptide of claim 66, wherein the polypeptide comprises a glutamic acid or aspartic acid at position 33.

68. The polypeptide of claim 66, wherein the polypeptide comprises a glutamic acid at position 33 and a tyrosine or phenylalanine at position 8.

69. The polypeptide of any one of claims 50-68, wherein the polypeptide comprises a glutamic acid at position 17.

70. A polypeptide that binds IL-2 receptor beta (IL-2Rβ) and comprises an amino acid sequence at least 80% identical to the amino acid sequence of SEQ ID NO: 13:
(SEQ ID NO: 13)
KIQLYAEHAR YDAEMILNIV KTNSPPAEEE LEDYAFNFEL
ILEEIARLFE SGDQKDEAEK AKRMKEWMKR IKTTASEDEQ
EEMANAIITI LTSWIRS,
wherein the polypeptide comprises:
a glutamic acid or aspartic acid at position 17,
a glutamic acid, aspartic acid, threonine, or serine at position 95,
an arginine or lysine at position 99, and
wherein the position numbering is according to SEQ ID NO: 13, provided that the lysine at the N terminus of SEQ ID NO: 13 is designated as position 4.

71. The polypeptide of claim 70, wherein the polypeptide comprises an amino acid sequence at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 13.

72. The polypeptide of any one of claims 50-71, wherein the polypeptide comprises an arginine at position 99.

73. The polypeptide of any one of claims 50-72, wherein the polypeptide comprises a glutamic acid at position 17.

74. The polypeptide of any one of claims 50-73, wherein the polypeptide comprises a glutamic acid or threonine at position 95.

75. The polypeptide of claim 74, wherein the polypeptide comprises a glutamic acid at position 95.

76. The polypeptide of claim 74, wherein the polypeptide comprises a threonine at position

95.

77. The polypeptide of any one of claims 50-76, wherein the polypeptide comprises a leucine, isoleucine, valine, arginine, or lysine at position 13.

78. The polypeptide of claim 77, wherein the polypeptide comprises a leucine or arginine at position 13.

79. The polypeptide of claim 78, wherein the polypeptide comprises a leucine at position 13.

80. The polypeptide of claim 78, wherein the polypeptide comprises an arginine at position 13.

81. The polypeptide of any one of claims 50-80, wherein no amino acids are added or deleted in the region from position 4 to position 22, position 33 to position 55, and position 80 to position 100.

82. The polypeptide of claim 81, wherein no amino acids are added or deleted in the region from position 58 to position 76.

83. The polypeptide of any one of claims 1-82, wherein the polypeptide comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 9:
(SEQ ID NO: 9)
PKKKIQLYAE HALYDAEMIL KIVKTNSPPA EEELEDYAFN
FELILEEIAR LFESGDQKDE AEKAKRMKEW MKRIKTTASE
DEQEEMANRI RKILEEWIRS.

84. The polypeptide of any one of claims 1-82, wherein the polypeptide comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 10:
(SEQ ID NO: 10)
PKKKIQLFAE HALYDAEMIL KIVKTNSPPA EEELEDYAFN
FELILEEIAR LFESGDQKDE AEKAKRMKEW MKRIKTTASE
DEQEEMANKI RKILEEWIRS.

85. The polypeptide of any one of claims 1-82, wherein the polypeptide comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% identical to an amino acid sequence selected from SEQ ID NOs: 11-15, 37, and 39.
(SEQ ID NO: 11)
KIQLHAEHAR YDAEMILNIV KTNSPPAEEK LEDYAFNFEL
ILEEIARLFE SGDQKDEAEK AKRMKEWMKR IKTTASEDEQ
EEMANAIITI LTSWIRS;
(SEQ ID NO: 12)
KIQLHAEHAL YDAEMILKIV KTNSPPAEEK LEDYAFNFEL
ILEEIARLFE SGDQKDEAEK AKRMKEWMKR IKTTASEDEQ
EEMANRIRKI LEEWIRS;
(SEQ ID NO: 13)
KIQLYAEHAR YDAEMILNIV KTNSPPAEEE LEDYAFNFEL
ILEEIARLFE SGDQKDEAEK AKRMKEWMKR IKTTASEDEQ
EEMANAIITI LTSWIRS;
(SEQ ID NO: 14)
PKKKIQLHAE HARYDAEMIL NIVKTNSPPA EEKLEDYAFN
FELILEEIAR LFESGDQKDE AEKAKRMKEW MKRIKTTASE
DEQEEMANAI ITILTSWIRS;
(SEQ ID NO: 15)
PKKKIQLHAE HALYDAEMIL KIVKTNSPPA EEKLEDYAFN
FELILEEIAR LFESGDQKDE AEKAKRMKEW MKRIKTTASE
DEQEEMANRI RKILEEWIRS;
(SEQ ID NO: 37)
KIQLHAE HALYDAEMIL KIVKTNSPPA EEKLEDYAFN
FELILEEIAR LFESGDQKDE AEKAKRMKEW MKRIKTTASE
DEQEEMANKI RKILEEWIRS;
(SEQ ID NO: 39)
PKKKIQLHAE HALYDAEMIL KIVKTNSPPA EEKLEDYAFN
FELILEEIAR LFESGDQKDE AEKAKRMKEW MKRIKTTASE
DEQEEMANKI RKILEEWIRS;
(SEQ ID NO: 16)
PKKKIQLYAE HARYDAEMIL NIVKTNSPPA EEELEDYAFN
FELILEEIAR LFESGDOKDE AEKAKRMKEW MKRIKTTASE
DEQEEMANAI ITILTSWIRS.

86. A polypeptide comprising an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to the amino acid sequence of SEQ ID NO: 9:
(SEQ ID NO: 9)
PKKKIQLYAE HALYDAEMIL KIVKTNSPPA EEELEDYAFN
FELILEEIAR LFESGDQKDE AEKAKRMKEW MKRIKTTASE
DEQEEMANRI RKILEEWIRS
wherein the polypeptide comprises a glutamic acid at position 17, a glutamic acid, threonine, or serine at position 95, and an arginine at position 99, wherein the position numbering is according to SEQ ID NO:9; wherein the polypeptide binds IL-2 receptor beta (IL-2Rβ); and wherein the polypeptide binds IL-2 receptor common gamma (IL-2Rγc) with lower affinity than IL-2, or wherein the polypeptide does not bind IL-2Rγc.

87. The polypeptide of claim 86 wherein the polypeptide comprises a leucine at position 13, a lysine at position 21, an arginine at position 89, an arginine at position 91, a lysine at position 92, and a glutamic acid at position 96.

88. The polypeptide of claim 86 or 87 wherein the polypeptide comprises a glutamic acid at position 95.

89. A polypeptide comprising an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to the amino acid sequence of SEQ ID NO: 16:
(SEQ ID NO: 16)
PKKKIQLYAE HARYDAEMIL NIVKTNSPPA EEELEDYAFN
FELILEEIAR LFESGDQKDE AEKAKRMKEW MKRIKTTASE
DEQEEMANAI ITILTSWIRS
wherein the polypeptide comprises a glutamic acid at position 17, a glutamic acid, threonine, or serine at position 95, and an arginine at position 99, wherein the position numbering is according to SEQ ID NO: 16, wherein the polypeptide binds IL-2 receptor beta (IL-2Rβ); and wherein the polypeptide binds IL-2 receptor common gamma (IL-2Rγc) with lower affinity than IL-2, or wherein the polypeptide does not bind IL-2Rγc.

90. The polypeptide of claim 89 wherein the polypeptide comprises an arginine at position 13.

91. The polypeptide of claim 89 or 90 wherein the polypeptide comprises a threonine at position 95.

92. A polypeptide that binds IL-2 receptor beta (IL-2Rβ) and comprises the amino acid sequence of SEQ ID NO: 17:
(SEQ ID NO: 17)
KIQLYAEHAL YDAX₁₇MILKIV KTNSPPAEEE LEDYAFNFEL
ILEEIARLFE SGDQKDEAEK AKRMKEWMKR IKTTASEDEQE
EMANX₈₉IX₉₁X₉₂I LX₉₅X₉₆WIX₉₉S
wherein:
X₁₇ is glutamic acid or aspartic acid;
X₈₉ is arginine or lysine;
X₉₁ is arginine or lysine;
X₉₂ is arginine or lysine;
X₉₅ is threonine, serine, glutamic acid, or aspartic acid;
X₉₆ is aspartic acid or glutamic acid; and
X₉₉ is arginine or lysine
wherein the polypeptide contains a total of no more than ten, no more than nine, no more than eight, no more than seven, no more than six, no more than five, no more than four, or no more than three substitutions, additions, and/or deletions at amino acid positions in SEQ ID NO: 17 not designated as X.

93. A polypeptide that binds IL-2 receptor beta (IL-2Rβ) and comprises the amino acid sequence of SEQ ID NO: 18:
(SEQ ID NO: 18)
KIQLFAEHAL YDAX₁₇MILKIV KTNSPPAEEE LEDYAFNFEL
ILEEIARLFE SGDQKDEAEK AKRMKEWMKR IKTTASEDEQE
EMANX₈₉IX₉₁X₉₂I LX₉₅X₉₆WIX₉₉S
wherein:
X₁₇ is glutamic acid or aspartic acid;
X₈₉ is arginine or lysine;
X₉₁ is arginine or lysine;
X₉₂ is arginine or lysine;
X₉₅ is threonine, serine, glutamic acid, or aspartic acid;
X₉₆ is aspartic acid or glutamic acid; and
X₉₉ is arginine or lysine
wherein the polypeptide contains a total of no more than ten, no more than nine, no more than eight, no more than seven, no more than six, no more than five, no more than positions in SEQ ID NO: 18 not designated as X.

94. The polypeptide of claim 92 or 93 comprising (i) an asparagine or lysine at position 21, (ii) a cysteine, tyrosine, glutamic acid or aspartic acid at position 33 and/or (iii) a tyrosine, or phenylalanine at position 8, or any combination thereof, wherein the position numbering is according to SEQ ID NO:17 or 18, provided that the lysine at the N terminus of SEQ ID NO: 17 and 18 is designated as position 4.

95. The polypeptide of claim 94 comprising a cysteine, tyrosine, glutamic acid or aspartic acid at position 33.

96. The polypeptide of claim 94 comprising a glutamic acid at position 33.

97. The polypeptide of any one of claims 94-96 comprising a lysine at position 21.

98. A polypeptide that binds IL-2 receptor beta (IL-2Rβ) and comprises the amino acid sequence of SEQ ID NO: 19:
(SEQ ID NO: 19)
KIQLX₈AEHAL YDAX₁₇MILX₂₁IV KTNSPPAEEX₃₃
LEDYAFNFEL ILEEIARLEE SGDQKDEAEK AKRMKEWMKR
IKTTASEDEQE EMANX₈₉IX₉₁X₉₂I LX₉₅X₉₆WIX₉₉S
wherein:
X₈, X₂₁ and X₃₃ are amino acids;
X₁₇ is glutamic acid or aspartic acid;
X₈₉ is arginine or lysine;
X₉₁ is arginine or lysine;
X₉₂ is arginine or lysine;
X₉₅ is threonine, serine, glutamic acid, or aspartic acid;
X₉₆ is aspartic acid or glutamic acid; and
X₉₉ is arginine or lysine
wherein the polypeptide contains a total of no more than ten, no more than nine, no more than eight, no more than seven, no more than six, no more than five, no more than positions in SEQ ID NO: 19 not designated as X.

99. The polypeptide of claim 98, wherein:
X₈ is histidine, tyrosine, or phenylalanine.

100. The polypeptide of claim 99, wherein:
X₈ is tyrosine or phenylalanine.

101. The polypeptide of any one of claims 98-100, wherein:
X₂₁ is asparagine or lysine.

102. The polypeptide of claim 101, wherein:
X₂₁ is lysine.

103. The polypeptide of any one of claims 98-102, wherein:
X₃₃ is cysteine, tyrosine, glutamic acid or aspartic acid.

104. The polypeptide of claim 103, wherein:
X₃₃ is glutamic acid.

105. The polypeptide of any one of claims 92-104, wherein:
X₉₁ is arginine;
X₉₂ is lysine; and
X₉₆ is glutamic acid.

106. A polypeptide that binds IL-2 receptor beta (IL-2Rβ) and comprises the amino acid sequence of SEQ ID NO: 33:
(SEQ ID NO: 33)
KIQLYAEHAL YDAX₁₇MILKIV KTNSPPAEEE LEDYAFNFEL
ILEEIARLFE SGDQKDEAEK AKRMKEWMKR IKTTASEDEQE
EMANRIRKI LX₉₅EWIX₉₉S
wherein:
X₁₇ is glutamic acid or aspartic acid;
X₉₅ is threonine, serine, glutamic acid, or aspartic acid;
X₉₉ is arginine or lysine,
wherein the polypeptide contains a total of no more than ten, no more than nine, no more than eight, no more than seven, no more than six, no more than five, no more than four, or no more than three substitutions, additions, and/or deletions at amino acid positions in SEQ ID NO: 33 not designated as X.

107. A polypeptide that binds IL-2 receptor beta (IL-2Rβ) and comprises the amino acid sequence of SEQ ID NO: 45:
(SEQ ID NO: 45)
KIQLYAEHAL YDAEMILKIV KTNSPPAEEE LEDYAFNFEL
ILEEIARLFE SGDQKDEAEK AKRMKEWMKR IKTTASEDEQE
EMANRIRKI LX₉₅EWIX₉₉S
wherein:
X₉₅ is threonine, serine, glutamic acid, or aspartic acid; and
X₉₉ is arginine or lysine,
wherein the polypeptide contains a total of no more than ten, no more than nine, no more than eight, no more than seven, no more than six, no more than five, no more than four, or no more than three substitutions, additions, and/or deletions at amino acid positions in SEQ ID NO: 45 not designated as X.

108. A polypeptide that binds IL-2 receptor beta (IL-2Rβ) and comprises the amino acid sequence of SEQ ID NO: 46:
(SEQ ID NO: 46)
KIQLFAEHAL YDAEMILKIV KTNSPPAEEE LEDYAFNFEL
ILEEIARLFE SGDQKDEAEK AKRMKEWMKR IKTTASEDEQE
EMANKIRKI LX₉₅EWIX₉₉S
wherein:
X₉₅ is threonine, serine, glutamic acid, or aspartic acid; and
X₉₉ is arginine or lysine,
wherein the polypeptide contains a total of no more than ten, no more than nine, no more than eight, no more than seven, no more than six, no more than five, no more than positions in SEQ ID NO: 46 not designated as X.

109. A polypeptide that binds IL-2 receptor beta (IL-2Rβ) and comprises the amino acid sequence of SEQ ID NO: 36:
(SEQ ID NO: 36)
KIQLFAEHAL YDAX₁₇MILKIV KTNSPPAEEE LEDYAFNFEL
ILEEIARLFE SGDQKDEAEK AKRMKEWMKR IKTTASEDEQE
EMANKIRKI LX₉₅EWIX₉₉S
wherein:
X₁₇ is glutamic acid or aspartic acid;
X₉₅ is threonine, serine, glutamic acid, or aspartic acid; and
X₉₉ is arginine or lysine,
wherein the polypeptide contains a total of no more than ten, no more than nine, no more than eight, no more than seven, no more than six, no more than five, no more than four, or no more than three substitutions, additions, and/or deletions at amino acid positions in SEQ ID NO: 36 not designated as X.

110. The polypeptide of any one of claims 106-109 wherein one, two, three, or four of the following are true: if there is a substitution at position 89 it is to arginine or lysine; if there is a substitution at position 91 it is to lysine; if there is a substitution at position 92, it is to arginine; and/or if there is a substitution at position 96, it is to aspartic acid, wherein the position numbering is according to SEQ ID NO: 33, 36, 45 or 46 provided that the lysine at the N terminus of SEQ ID NO: 33, 36, 45 or 46 is designated as position 4.

111. The polypeptide of any one of claims 106-109 wherein if there is a substitution at position 91 it is to lysine; if there is a substitution at position 92, it is to arginine; and if there is a substitution at position 96, it is to aspartic acid, wherein the position numbering is according to SEQ ID NO: 33, 36, 45 or 46 provided that the lysine at the N terminus of SEQ ID NO: 33, 36, 45 or 46 is designated as position 4.

112. The polypeptide of any one of claims 106-109, wherein there are no substitutions at positions 91, 92, and 96 wherein the position numbering is according to SEQ ID NO: 33, 36, 45 or 46, provided that the lysine at the N terminus of SEQ ID NO: 33, 36, 45 or 46 is designated as position 4.

113. The polypeptide of any one of claims 106-112, wherein there are no substitutions at position 89 wherein the position numbering is according to SEQ ID NO: 33, 36, 45 or 46, provided that the lysine at the N terminus of SEQ ID NO: 33, 36, 45 or 46 is designated as position 4.

114. The polypeptide of any one of claims 106-113, wherein:
if there is a substitution at position 8, it is a substitution to tyrosine, histidine or phenylalanine, wherein the position numbering is according to SEQ ID NO: 33, 36, 45 or 46, provided that the lysine at the N terminus of SEQ ID NO: 33, 36, 45 or 46 is designated as position 4.

115. The polypeptide of claim 114, wherein:
if there is a substitution at position 8, it is a substitution to tyrosine or phenylalanine, wherein the position numbering is according to SEQ ID NO: 33, 36, 45 or 46, provided that the lysine at the N terminus of SEQ ID NO: 33, 36, 45 or 46 is designated as position 4.

116. The polypeptide of any one of claims 106-115 wherein:
if there is a substitution of the glutamic acid at position 33, it is a substitution to cysteine, aspartic acid, lysine, or tyrosine, wherein the position numbering is according to SEQ ID NO: 33, 36, 45 or 46, provided that the lysine at the N terminus of SEQ ID NO: 33, 36, 45 or 46 is designated as position 4.

117. The polypeptide of claim 112 wherein:
if there is a substitution of the glutamic acid at position 33, it is a substitution to cysteine, aspartic acid, or tyrosine, wherein the position numbering is according to SEQ ID NO: 33, 36, 45 or 46, provided that the lysine at the N terminus of SEQ ID NO: 33, 36, 45 or 46 is designated as position 4.

118. The polypeptide of any one of claims 92-117, wherein X₁₇ is glutamic acid and X₉₉ is arginine.

119. The polypeptide of any one of claims 92-118, wherein:
X₉₅ is threonine, glutamic acid, or aspartic acid.

120. The polypeptide of claim 119 wherein:
X₉₅ is threonine or glutamic acid.

121. The polypeptide of claim 119 wherein:
X₉₅ is glutamic acid.

122. The polypeptide of any one of claims 92-121, wherein no amino acids are added or deleted in the region from position 4 to position 22, position 33 to position 55, and position 80 to position 100 of SEQ ID NO: 17, 18, 19, 33, 36, 45 or 46 provided that the lysine at the N terminus of of SEQ ID NO: 17, 18, 19, 33, 36, 45 or 46 is designated as position 4.

123. The polypeptide of claim 122, wherein no amino acids are added or deleted in the region from position 58 to position 76 SEQ ID NO: 17, 18, or 19, 33, 36, 45 or 46.

124. The polypeptide of any one of claims 1-123 wherein the polypeptide is isolated.

125. The polypeptide of any one of claims 1-124, wherein the polypeptide binds IL-2 receptor common gamma (IL-2Rγc) in the presence of IL-2Rβ with lower affinity than does IL-2, or wherein the polypeptide does not detectably bind IL-2Rγc in the presence of IL-2Rβ.

126. The polypeptide of any one of claims 1-124, wherein the polypeptide binds IL-2 receptor common gamma (IL-2Ryc) in the presence of IL-2Rβ with at least 5 fold, at least 10 fold, at least 100 fold, at least 1000 fold, or at least 10,000 fold lower affinity than does IL-2.

127. The polypeptide of any one of claims 1-124, wherein the polypeptide does not detectably bind IL-2Rγc in the presence of IL-2Rβ.

128. The polypeptide of claim 125 or 126, wherein the polypeptide binds IL-2 receptor beta (IL-2Rβ) with greater affinity than does IL-2.

129. The polypeptide of any one of claims 1-128, wherein the polypeptide binds IL-2 receptor common gamma (IL-2Rγc) in the presence of IL-2Rβ with at least 5 fold, at least 10 fold, at least 100 fold, at least 1000 fold, or at least 10,000 fold lower affinity than does Neo-2/15; and wherein the polypeptide binds IL-2 receptor beta (IL-2Rβ) with greater affinity than does Neo-2/15.

130. The polypeptide of claim 129, wherein the polypeptide does not detectably bind IL-2Rγc in the presence of IL-2Rβ; and wherein the polypeptide binds IL-2 receptor beta (IL-2Rβ) with greater affinity than does Neo-2/15.

131. The polypeptide of any one of claims 1-130, wherein the polypeptide binds IL-2Rβ with a KD of 20 nM or lower.

132. The polypeptide of any one of claims 1-130, wherein the polypeptide binds IL-2Rβ with a KD of 10 nM or lower.

133. The polypeptide of any one of claims 1-130, wherein the polypeptide binds IL-2Rβ with a KD of 5 nM or lower.

134. The polypeptide of any one of claims 1-130, wherein the polypeptide binds IL-2Rβ with a KD of 1 nM or lower.

135. The polypeptide of any one of claims 131-134, wherein KD is measured using biolayer interferometry.

136. The polypeptide of any one of claims 1-135, wherein the polypeptide inhibits IL-2 binding to the IL-2 receptor βγ_{c} heterodimer (IL-2βγ_{c}) in vitro and/or in vivo.

137. The polypeptide of claim 136 wherein the polypeptide inhibits IL-2 binding to the IL-2 receptor βγ_{c} heterodimer (IL-2βγ_{c}) by at least about 30%, at least about 40%, at least about 50%, at least about 60%, at least about 70%, at least about 80%, at least about 90%, or at least about 95%.

138. The polypeptide of claim 136 or 137 wherein the polypeptide inhibits IL-2 binding to the IL-2 receptor βγ_{c} heterodimer in CD8 positive and CD4 positive T cells.

139. The polypeptide of claim 136 or 137 wherein the polypeptide inhibits IL-2 binding to the IL-2 receptor βγ_{c} heterodimer in NK cells.

140. The polypeptide of any one of claims 1-135, wherein the polypeptide inhibits IL-2 binding to IL-2 receptor β in vitro and/or in vivo.

141. The polypeptide of claim 140 wherein the polypeptide inhibits IL-2 binding to IL-2 receptor β by at least about 30%, at least about 40%, at least about 50%, at least about 60%, at least about 70%, at least about 80%, at least about 90%, or at least about 95%.

142. The polypeptide of claim 140 or 141 wherein the polypeptide inhibits IL-2 binding to IL-2 receptor β in CD8 positive and CD4 positive T cells.

143. The polypeptide of claim 140 or 141 wherein the polypeptide inhibits IL-2 binding to IL-2 receptor β in NK cells.

144. The polypeptide of any one of claims 1-143, wherein the polypeptide inhibits IL-2 signaling in vitro and/or in vivo.

145. The polypeptide of claim 144, wherein the polypeptide inhibits IL-2 signaling by at least 60%, at least 70%, at least 80%, at least 85%, at least 90% less, or at least 95% in IL-2Rβγc positive cells.

146. The polypeptide of claim 145, wherein inhibiton of IL-2 signaling is measured by STATS phosphorylation.

147. The polypeptide of any one of claims 144-146, wherein the polypeptide inhibits IL-2 signaling in CD8 positive T cells, CD4 positive T cells, and NK cells.

148. The polypeptide of any one of claims 1-147, wherein the polypeptide stimulates STATS phosphorylation at level that is at least 50% less, at least 60% less, at least 70% less, at least 80% less, at least 85% less, at least 90% less, or at least 95% less than the level that IL-2 stimulates STATS phosphorylation in the same cell type.

149. The polypeptide of any of the previous claims, wherein the polypeptide inhibits the binding of IL-2 to the IL-2 receptor and/or signaling via the IL-2 receptor by at least about 30%, at least about 40%, at least about 50%, at least about 60%, at least about 70%, at least about 80%, at least about 90%, or at least about 95% in IL-2Rβ positive cells that are IL-2Rα negative and by not more than 50%, not more than 30% or by not more than 20% in IL-2Rβ positive cells that are IL-2Rα positive.

150. The polypeptide of any one of claims 1 to 148, wherein the polypeptide inhibits the ability of IL-2 to stimulate STATS phosphorylation by at least about 30%, at least about 40%, at least about 50%, at least about 60%, at least about 70%, at least about 80%, at least about 90%, or at least about 95% in IL-2Rβ positive cells that are IL-2Rα negative and by not more than 50%, not more than 30% or by not more than 20% in IL-2Rβ positive cells that are IL-2Rα positive.

151. The polypeptide of any one of claims 1 to 150 wherein the polypeptide comprises a targeting agent.

152. The polypeptide of any one of the preceding claims wherein the polypeptide comprises a stabilizing agent.

153. The polypeptide of claim 152 wherein the stabilizing agent is a Fc region of an antibody.

154. The polypeptide of any one of the preceding claims wherein the IL-2 receptor is a human IL-2 receptor.

155. A pharmaceutical composition comprising a polypeptide of any one of the preceding claims and a pharmaceutically acceptable carrier or diluent.

156. An isolated polynucleotide comprising a polynucleotide sequence that encodes a polypeptide of any one of claims 1-155.

157. A vector comprising the polynucleotide of claim 156.

158. An isolated host cell comprising the vector of claim 157.

159. An isolated host cell that expresses the polypeptide of any one of claims 1-154.

160. A method of producing a polypeptide of any one of claims 1-154 comprising incubating the host cell of claim 158 or 159 under conditions suitable for expressing the polypeptide.

161. The method of claim 160, further comprising isolating the polypeptide.

162. A method for antagonizing the IL-2 receptor in a subject comprising administering to the subject a polypeptide of any one of claims 1-154 or the pharmaceutical composition of claim 155.

163. A method for modulating IL-2 activity in a subject comprising administering to the subject the polypeptide of any one of claims 1-154 or the pharmaceutical composition of claim 155.

164. A method for treating disease associated with IL-2 and/or IL-15 activity in a subject comprising administering to the subject the polypeptide of any one of claims 1-154 or the pharmaceutical composition of claim 155.

165. The method of claim 164, wherein the disesase associated with IL-2 and/or IL-15 activity is an autoimmune disease.

166. The method of claim 163 or claim 164, whethein the subject is suffering from an autoimmune disease.

167. The method of claim 165 or claim 166, wherein the autoimmune disease is selected from the group consisting of a rheumatic disease, including, but not limited to, rheumatoid arthritis, systemic lupus erythematosus, Sjogren's syndrome, scleroderma, mixed connective tissue disease, dermatomyositis, polymyositis, Reiter's syndrome or Behcet's disease; type II diabetes; an autoimmune disease of the thyroid, including, but not limited, Hashimoto's thyroiditis or Graves' Disease; an autoimmune disease of the central nervous system, including, but not limited to, multiple sclerosis, myasthenia gravis, or encephalomyelitis; (5) phemphigus, including but not limited to, phemphigus vulgaris, phemphigus vegetans, phemphigus foliaceus, Senear-Usher syndrome, or Brazilian phemphigus; psoriasis; inflammatory bowel disease, including, but not limited to ulcerative colitis or Crohn's Disease; and celiac disease.

168. A method for treating a subject who is suffering from an autoimmune disease, comprising administering to the subject the polypeptide of any one of claims 1-154 or the pharmaceutical composition of claim 155.

169. The method of claim 168, wherein the autoimmune disease is selected from the group consisting of a rheumatic disease, including, but not limited to, rheumatoid arthritis, systemic lupus erythematosus, Sjogren's syndrome, scleroderma, mixed connective tissue disease, dermatomyositis, polymyositis, Reiter's syndrome or Behcet's disease; type II diabetes; an autoimmune disease of the thyroid, including, but not limited, Hashimoto's thyroiditis or Graves' Disease; an autoimmune disease of the central nervous system, including, but not limited to, multiple sclerosis, myasthenia gravis, or encephalomyelitis; (5) phemphigus, including but not limited to, phemphigus vulgaris, phemphigus vegetans, phemphigus foliaceus, Senear-Usher syndrome, or Brazilian phemphigus; psoriasis; inflammatory bowel disease, including, but not limited to ulcerative colitis or Crohn's Disease; and celiac disease

170. A method for treating a subject who has received a transplant of biological materials, such as an organ, tissue, or cell transplant comprising administering to the subject the polypeptide of any one of claims 1-154 or the pharmaceutical composition of claim 155

171. The method of any one of claims 162-170 wherein the subject is human.


SEQUENCE LISTING (49 sequences)

SEQ ID NO: 1 - protein, 19 aa, Artificial Sequence
  Synthetic
  misc_feature: See specification as filed for detailed description of substitutions and preferred embodiments
  misc_feature (14)..(14): X is glutamic acid or aspartic acid
  misc_feature (18)..(18): X is any amino acid
KIQLYAEHALYDAXMILXI

SEQ ID NO: 2 - protein, 23 aa, Artificial Sequence
  Synthetic
ELEDYAFNFELILEEIARLFESG

SEQ ID NO: 3 - protein, 21 aa, Artificial Sequence
  Synthetic
  misc_feature: See specification as filed for detailed description of substitutions and preferred embodiments
  misc_feature (10)..(10): X is arginine or lysine
  misc_feature (12)..(12): X is arginine or lysine
  misc_feature (13)..(13): X is arginine or lysine
  misc_feature (16)..(16): X is threonine, serine, glutamic acid, or aspartic acid
  misc_feature (17)..(17): X is aspartic acid or glutamic acid
  misc_feature (20)..(20): X is arginine or lysine
EDEQEEMANXIXXILXXWIXS

SEQ ID NO: 4 - protein, 19 aa, Artificial Sequence
  Synthetic
  misc_feature: See specification as filed for detailed description of substitutions and preferred embodiments
  misc_feature (5)..(5): X is any amino acid
  misc_feature (14)..(14): X is glutamic acid or aspartic acid
  misc_feature (18)..(18): X is any amino acid
KIQLXAEHALYDAXMILXI

SEQ ID NO: 5 - protein, 23 aa, Artificial Sequence
  Synthetic
  misc_feature: See specification as filed for detailed description of substitutions and preferred embodiments
  misc_feature (1)..(1): X is any amino acid
XLEDYAFNFELILEEIARLFESG

SEQ ID NO: 6 - protein, 19 aa, Artificial Sequence
  Synthetic
KDEAEKAKRMKEWMKRIKT

SEQ ID NO: 7 - protein, 97 aa, Artificial Sequence
  Synthetic
KIQLYAEHALYDAEMILKIVKTNSPPAEEELEDYAFNFELILEEIARLFESGDQKDEAEKAKRMKEWMKRIKTTASEDEQEEMANRIRKILEEWIRS

SEQ ID NO: 8 - protein, 97 aa, Artificial Sequence
  Synthetic
KIQLFAEHALYDAEMILKIVKTNSPPAEEELEDYAFNFELILEEIARLFESGDQKDEAEKAKRMKEWMKRIKTTASEDEQEEMANKIRKILEEWIRS

SEQ ID NO: 9 - protein, 100 aa, Artificial Sequence
  Synthetic
PKKKIQLYAEHALYDAEMILKIVKTNSPPAEEELEDYAFNFELILEEIARLFESGDQKDEAEKAKRMKEWMKRIKTTASEDEQEEMANRIRKILEEWIRS

SEQ ID NO: 10 - protein, 100 aa, Artificial Sequence
  Synthetic
PKKKIQLFAEHALYDAEMILKIVKTNSPPAEEELEDYAFNFELILEEIARLFESGDQKDEAEKAKRMKEWMKRIKTTASEDEQEEMANKIRKILEEWIRS

SEQ ID NO: 11 - protein, 97 aa, Artificial Sequence
  Synthetic
KIQLHAEHARYDAEMILNIVKTNSPPAEEKLEDYAFNFELILEEIARLFESGDQKDEAEKAKRMKEWMKRIKTTASEDEQEEMANAIITILTSWIRS

SEQ ID NO: 12 - protein, 97 aa, Artificial Sequence
  Synthetic
KIQLHAEHALYDAEMILKIVKTNSPPAEEKLEDYAFNFELILEEIARLFESGDQKDEAEKAKRMKEWMKRIKTTASEDEQEEMANRIRKILEEWIRS

SEQ ID NO: 13 - protein, 97 aa, Artificial Sequence
  Synthetic
KIQLYAEHARYDAEMILNIVKTNSPPAEEELEDYAFNFELILEEIARLFESGDQKDEAEKAKRMKEWMKRIKTTASEDEQEEMANAIITILTSWIRS

SEQ ID NO: 14 - protein, 100 aa, Artificial Sequence
  Synthetic
PKKKIQLHAEHARYDAEMILNIVKTNSPPAEEKLEDYAFNFELILEEIARLFESGDQKDEAEKAKRMKEWMKRIKTTASEDEQEEMANAIITILTSWIRS

SEQ ID NO: 15 - protein, 100 aa, Artificial Sequence
  Synthetic
PKKKIQLHAEHALYDAEMILKIVKTNSPPAEEKLEDYAFNFELILEEIARLFESGDQKDEAEKAKRMKEWMKRIKTTASEDEQEEMANRIRKILEEWIRS

SEQ ID NO: 16 - protein, 100 aa, Artificial Sequence
  Synthetic
PKKKIQLYAEHARYDAEMILNIVKTNSPPAEEELEDYAFNFELILEEIARLFESGDQKDEAEKAKRMKEWMKRIKTTASEDEQEEMANAIITILTSWIRS

SEQ ID NO: 17 - protein, 97 aa, Artificial Sequence
  Synthetic
  misc_feature: See specification as filed for detailed description of substitutions and preferred embodiments
  misc_feature (14)..(14): X is glutamic acid or aspartic acid
  misc_feature (86)..(86): X is arginine or lysine
  misc_feature (88)..(88): X is arginine or lysine
  misc_feature (89)..(89): X is arginine or lysine
  misc_feature (92)..(92): X is threonine, serine, glutamic acid, or aspartic acid
  misc_feature (93)..(93): X is aspartic acid or glutamic acid
  misc_feature (96)..(96): X is arginine or lysine
KIQLYAEHALYDAXMILKIVKTNSPPAEEELEDYAFNFELILEEIARLFESGDQKDEAEKAKRMKEWMKRIKTTASEDEQEEMANXIXXILXXWIXS

SEQ ID NO: 18 - protein, 97 aa, Artificial Sequence
  Synthetic
  misc_feature: See specification as filed for detailed description of substitutions and preferred embodiments
  misc_feature (14)..(14): X is glutamic acid or aspartic acid
  misc_feature (86)..(86): X is arginine or lysine
  misc_feature (88)..(88): X is arginine or lysine
  misc_feature (89)..(89): X is arginine or lysine
  misc_feature (92)..(92): X is threonine, serine, glutamic acid, or aspartic acid
  misc_feature (93)..(93): X is aspartic acid or glutamic acid
  misc_feature (96)..(96): X is arginine or lysine
KIQLFAEHALYDAXMILKIVKTNSPPAEEELEDYAFNFELILEEIARLFESGDQKDEAEKAKRMKEWMKRIKTTASEDEQEEMANXIXXILXXWIXS

SEQ ID NO: 19 - protein, 97 aa, Artificial Sequence
  Synthetic
  misc_feature: See specification as filed for detailed description of substitutions and preferred embodiments
  misc_feature (5)..(5): X is any amino acid
  misc_feature (14)..(14): X is glutamic acid or aspartic acid
  misc_feature (18)..(18): X is any amino acid
  misc_feature (30)..(30): X is any amino acid
  misc_feature (86)..(86): X is arginine or lysine
  misc_feature (88)..(88): X is arginine or lysine
  misc_feature (89)..(89): X is arginine or lysine
  misc_feature (92)..(92): X is threonine, serine, glutamic acid, or aspartic acid
  misc_feature (93)..(93): X is aspartic acid or glutamic acid
  misc_feature (96)..(96): X is arginine or lysine
KIQLXAEHALYDAXMILXIVKTNSPPAEEXLEDYAFNFELILEEIARLFESGDQKDEAEKAKRMKEWMKRIKTTASEDEQEEMANXIXXILXXWIXS

SEQ ID NO: 20 - protein, 19 aa, Artificial Sequence
  Synthetic
  misc_feature: See specification as filed for detailed description of substitutions and preferred embodiments
  misc_feature (10)..(10): X is arginine or lysine
  misc_feature (14)..(14): X is glutamic acid or aspartic acid
KIQLYAEHAXYDAXMILNI

SEQ ID NO: 21 - protein, 19 aa, Artificial Sequence
  Synthetic
  misc_feature: See specification as filed for detailed description of substitutions and preferred embodiments
  misc_feature (14)..(14): X is glutamic acid or aspartic acid
KIQLFAEHALYDAXMILKI

SEQ ID NO: 22 - protein, 21 aa, Artificial Sequence
  Synthetic
  misc_feature: See specification as filed for detailed description of substitutions and preferred embodiments
  misc_feature (16)..(16): X is threonine, serine, glutamic acid, or aspartic acid
  misc_feature (20)..(20): X is arginine or lysine
EDEQEEMANAIITILXSWIXS

SEQ ID NO: 23 - protein, 10 aa, Artificial Sequence
  Synthetic
VKTNSPPAEE

SEQ ID NO: 24 - protein, 3 aa, Artificial Sequence
  Synthetic
TAS

SEQ ID NO: 25 - protein, 100 aa, Artificial Sequence
  Synthetic
PKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 26 - protein, 153 aa, Homo sapiens
  misc_feature: amino acid sequence of native human wild-type IL-2, Genbank NP_000577.2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - protein, 19 aa, Artificial Sequence
  Synthetic
KIQLFAEHALYDAEMILKI

SEQ ID NO: 28 - protein, 66 aa, Artificial Sequence
  Synthetic
  misc_feature: See specification as filed for detailed description of substitutions and preferred embodiments
  misc_feature (1)..(1): X is any amino acid and can be present or absent
  misc_feature (2)..(2): X is any amino acid and can be present or absent
  misc_feature (3)..(3): X is any amino acid and can be present or absent
  misc_feature (8)..(8): X is Y or H
  misc_feature (13)..(13): X is R
  misc_feature (17)..(17): X is E
  misc_feature (21)..(21): X is N
  misc_feature (22)..(23): site of linker that can be present or absent
  misc_feature (23)..(23): X is E or K
  misc_feature (45)..(46): site of linker that can be present or absent, amino acid helical domain, and another linker that can be present or absent
  misc_feature (55)..(55): X is A
  misc_feature (57)..(57): X is I
  misc_feature (58)..(58): X is T
  misc_feature (61)..(61): X is E or T or S
  misc_feature (62)..(62): X is S
  misc_feature (65)..(65): X is R
  misc_feature (66)..(66): X is any amino acid and can be present or absent
XXXKIQLXAEHAXYDAXMILXIXLEDYAFNFELILEEIARLFESGEDEQEEMANXIXXILXXWIXX

SEQ ID NO: 29 - protein, 21 aa, Artificial Sequence
  Synthetic
  misc_feature: See specification as filed for detailed description of substitutions and preferred embodiments
  misc_feature (16)..(16): X is threonine, serine, glutamic acid, or aspartic acid
  misc_feature (20)..(20): X is arginine or lysine
EDEQEEMANKIRKILXEWIXS

SEQ ID NO: 30 - protein, 66 aa, Artificial Sequence
  Synthetic
  misc_feature: See specification as filed for detailed description of substitutions and preferred embodiments
  misc_feature (22)..(23): site of linker that can be present or absent
  misc_feature (45)..(46): site of linker that can be present or absent, amino acid helical domain, and another linker that can be present or absent
PKKKIQLHAEHARYDAEMILNIKLEDYAFNFELILEEIARLFESGEDEQEEMANAIITILTSWIRS

SEQ ID NO: 31 - protein, 66 aa, Artificial Sequence
  Synthetic
  misc_feature: See specification as filed for detailed description of substitutions and preferred embodiments
  misc_feature (22)..(23): site of linker that can be present or absent
  misc_feature (45)..(46): site of linker that can be present or absent, amino acid helical domain, and another linker that can be present or absent
PKKKIQLHAEHALYDAEMILKIKLEDYAFNFELILEEIARLFESGEDEQEEMANRIRKILEEWIRS

SEQ ID NO: 32 - protein, 19 aa, Artificial Sequence
  Synthetic
  misc_feature: See specification as filed for detailed description of substitutions and preferred embodiments
  misc_feature (14)..(14): X is glutamic acid or aspartic acid
KIQLYAEHALYDAXMILKI

SEQ ID NO: 33 - protein, 97 aa, Artificial Sequence
  Synthetic
  misc_feature: See specification as filed for detailed description of substitutions and preferred embodiments
  misc_feature (14)..(14): X is glutamic acid or aspartic acid
  misc_feature (92)..(92): X is threonine, serine, glutamic acid, or aspartic acid
  misc_feature (96)..(96): X is arginine or lysine
KIQLYAEHALYDAXMILKIVKTNSPPAEEELEDYAFNFELILEEIARLFESGDQKDEAEKAKRMKEWMKRIKTTASEDEQEEMANRIRKILXEWIXS

SEQ ID NO: 34 - protein, 66 aa, Artificial Sequence
  Synthetic
  misc_feature: See specification as filed for detailed description of substitutions and preferred embodiments
  misc_feature (22)..(23): site of linker that can be present or absent
  misc_feature (45)..(46): site of linker that can be present or absent, amino acid helical domain, and another linker that can be present or absent
PKKKIQLYAEHARYDAEMILNIELEDYAFNFELILEEIARLFESGEDEQEEMANAIITILTSWIRS

SEQ ID NO: 35 - protein, 66 aa, Artificial Sequence
  Synthetic
  misc_feature: See specification as filed for detailed description of substitutions and preferred embodiments
  misc_feature (22)..(23): site of linker that can be present or absent
  misc_feature (45)..(46): site of linker that can be present or absent, amino acid helical domain, and another linker that can be present or absent
PKKKIQLYAEHALYDAEMILKIELEDYAFNFELILEEIARLFESGEDEQEEMANRIRKILEEWIRS

SEQ ID NO: 36 - protein, 97 aa, Artificial Sequence
  Synthetic
  misc_feature: See specification as filed for detailed description of substitutions and preferred embodiments
  misc_feature (14)..(14): X is glutamic acid or aspartic acid
  misc_feature (92)..(92): X is threonine, serine, glutamic acid, or aspartic acid
  misc_feature (96)..(96): X is arginine or lysine
KIQLFAEHALYDAXMILKIVKTNSPPAEEELEDYAFNFELILEEIARLFESGDQKDEAEKAKRMKEWMKRIKTTASEDEQEEMANKIRKILXEWIXS

SEQ ID NO: 37 - protein, 97 aa, Artificial Sequence
  Synthetic
KIQLHAEHALYDAEMILKIVKTNSPPAEEKLEDYAFNFELILEEIARLFESGDQKDEAEKAKRMKEWMKRIKTTASEDEQEEMANKIRKILEEWIRS

SEQ ID NO: 38 - protein, 22 aa, Artificial Sequence
  Synthetic
  misc_feature: See specification as filed for detailed description of substitutions and preferred embodiments
  misc_feature (1)..(1): X is any amino acid and can be present or absent
  misc_feature (2)..(2): X is any amino acid and can be present or absent
  misc_feature (3)..(3): X is any amino acid and can be present or absent
XXXKIQLYAEHARYDAEMILNI

SEQ ID NO: 39 - protein, 100 aa, Artificial Sequence
  Synthetic
PKKKIQLHAEHALYDAEMILKIVKTNSPPAEEKLEDYAFNFELILEEIARLFESGDQKDEAEKAKRMKEWMKRIKTTASEDEQEEMANKIRKILEEWIRS

SEQ ID NO: 40 - protein, 21 aa, Artificial Sequence
  Synthetic
  misc_feature: See specification as filed for detailed description of substitutions and preferred embodiments
  misc_feature (16)..(16): X is serine, threonine, or glutamic acid
  misc_feature (21)..(21): X is any amino acid and can be present or absent
EDEQEEMANAIITILXSWIRX

SEQ ID NO: 41 - protein, 21 aa, Artificial Sequence
  Synthetic
  misc_feature: See specification as filed for detailed description of substitutions and preferred embodiments
  misc_feature (16)..(16): X is serine, threonine, or glutamic acid
  misc_feature (21)..(21): X is any amino acid and can be present or absent
EDEQEEMANRIRKILXEWIRX

SEQ ID NO: 42 - protein, 22 aa, Artificial Sequence
  Synthetic
  misc_feature: See specification as filed for detailed description of substitutions and preferred embodiments
  misc_feature (1)..(1): X is any amino acid and can be present or absent
  misc_feature (2)..(2): X is any amino acid and can be present or absent
  misc_feature (3)..(3): X is any amino acid and can be present or absent
XXXKIQLYAEHALYDAEMILKI

SEQ ID NO: 43 - protein, 21 aa, Artificial Sequence
  Synthetic
EDEQEEMANKIRKILEEWIRS

SEQ ID NO: 44 - protein, 19 aa, Artificial Sequence
  Synthetic
KIQLYAEHALYDAEMILKI

SEQ ID NO: 45 - protein, 97 aa, Artificial Sequence
  Synthetic
  misc_feature: See specification as filed for detailed description of substitutions and preferred embodiments
  misc_feature (92)..(92): X is threonine, serine, glutamic acid, or aspartic acid
  misc_feature (96)..(96): X is arginine or lysine
KIQLYAEHALYDAEMILKIVKTNSPPAEEELEDYAFNFELILEEIARLFESGDQKDEAEKAKRMKEWMKRIKTTASEDEQEEMANRIRKILXEWIXS

SEQ ID NO: 46 - protein, 97 aa, Artificial Sequence
  Synthetic
  misc_feature: See specification as filed for detailed description of substitutions and preferred embodiments
  misc_feature (92)..(92): X is threonine, serine, glutamic acid, or aspartic acid
  misc_feature (96)..(96): X is arginine or lysine
KIQLFAEHALYDAEMILKIVKTNSPPAEEELEDYAFNFELILEEIARLFESGDQKDEAEKAKRMKEWMKRIKTTASEDEQEEMANKIRKILXEWIXS

SEQ ID NO: 47 - protein, 21 aa, Artificial Sequence
  Synthetic
  misc_feature: See specification as filed for detailed description of substitutions and preferred embodiments
  misc_feature (16)..(16): X is threonine, serine, glutamic acid, or aspartic acid
  misc_feature (20)..(20): X is arginine or lysine
EDEQEEMANRIRKILXEWIXS

SEQ ID NO: 48 - protein, 369 aa, Homo sapiens
  misc_feature: amino acid sequence of the human IL-2 gamma receptor, Genbank accession locator NP_000197
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 49 - protein, 551 aa, Homo sapiens
  misc_feature: amino acid sequence of the human IL-2 beta receptor, Genbank accession locator NP_001333152.1
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV